(19) United States
(12) Patent Application Publication
(10) Pub. No.: US 2019/0040025 A1
(43) Pub. Date: Feb. 7, 2019

(54) INDOLEAMINE 2,3-DIOXYGENASE INHIBITOR, PREPARATION METHOD THEREFOR, AND APPLICATION

(71) Applicants: Jiangsu Hansoh Pharmaceutical Group Co., Ltd., Lianyungang, Jiangsu (CN); Shanghai Hansoh Biomedical Co., Ltd., Shanghai (CN)

(72) Inventors: Shenghua WU, Lianyungang, Jiangsu (CN); Kailong LI, Lianyungang, Jiangsu (CN); Rudi BAO, Lianyungang, Jiangsu (CN)

(21) Appl. No.: 16/093,821

(22) PCT Filed: Apr. 6, 2017
(86) PCT No.: PCT/CN2017/079585

§ 371 Date: Oct. 15, 2018

(30) Foreign Application Priority Data
Apr. 20, 2016 (CN) 201610246492.1
Jul. 20, 2016 (CN) 201610573473.X

(51) Int. Cl. C07D 271/08; C07D 413/04; A61K 31/4245; A61K 31/5377; A61P 35/00; A61K 45/06

(52) U.S. Cl. CPC C07D 271/08; C07D 413/04; A61K 45/06; A61K 31/5377; A61P 35/00; A61K 31/4245

(57) ABSTRACT

The present invention relates to an indoleamine 2,3-dioxygenase inhibitor having the structure of formula (I), a preparation method therefor, and an application. The IDO inhibitor is an N′-hydroxyl-N-phenylformamidine derivative, which has a high inhibitory activity on IDO, effectively inhibits IDO activity, and may also be used to inhibit patient immunosuppression. The inhibitor may be widely applied to treat or prevent cancers or tumors, viral infections, depression, neurodegenerative disorders, trauma, age-related cataracts, organ transplant rejection or autoimmune diseases, and has the potential to be developed into a new generation of immunosuppressors.

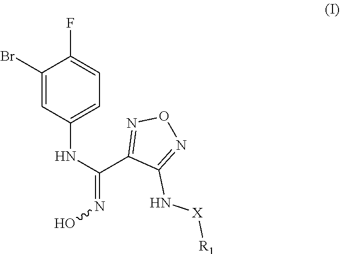

### CROSS-REFERENCE TO RELATED APPLICATIONS

This application is a Section 371 of International Application No. PCT/CN2017/079585, filed Apr. 6, 2017, which was published in the Chinese language on Oct. 26, 2017, under International Publication No. WO 2017/181849 A1, which claims priority under 35 U.S.C. § 119(b) to Chinese Application No. 201610246492.1, filed Apr. 20, 2016, and Chinese Application No. 201610573473.X, filed Jul. 20, 2016, the disclosures of which are incorporated herein by reference in their entirety.

### FIELD OF THE INVENTION

The invention belongs to the field of drug development, in particular relates to an indoleamine 2,3-dioxygenase inhibitor, a preparation method and application thereof.

### BACKGROUND OF THE INVENTION

Indoleamine 2,3-dioxygenase (IDO) is a protease involved in tryptophan metabolism. Tryptophan is one of the eight essential amino acids. Tryptophan can be used to synthesize proteins in vivo. Tryptophan can also be used as a precursor substrate to synthesize 5-hydroxytryptamine and melatonin (N-acetyl-5-methoxytryptamine) through the methoxyindole metabolic pathway. 5-Hydroxytryptamine and melatonin are neurotransmitters and neuroendocrine hormones involved in the regulation of various neurological and physiological processes in the body. In addition, tryptophan can also produce metabolites such as kynurenine through the kynurenine metabolic pathway. The first step in the kynurenine metabolic pathway is the degradation of tryptophan L-tryptophan to N-formyl-kynurenine under the catalyst is of indoleamine 2,3-dioxygenase or tryptophan 2,3-dioxygenase (TDO). N-formyl-kynurenine forms kynurenine under the catalysis of kynurenine formamide, and then kynurenine can also be further metabolized to form 3-hydroxyanthranilic acid, quinolinic acid, and picolinic acid. Quinolinic acid is neurotoxic, while picolinic acid has neuroprotective effects. Kynurenine and 3-hydroxyanthranilic acid are involved in the regulation of lymphocyte activity, thereby leading to the inhibition of the immune system.

With the exception of placental tissue, indoleamine 2,3-dioxygenase is not expressed in most tissue cells under normal health conditions. In the region of inflammation, inflammatory cytokines such as interferon gamma can induce an increased expression of indoleamine 2,3-dioxygenase. Various experimental results have proved that the high expression of indoleamine 2,3-dioxygenase in tissue cells can lead to the inhibition of the immune system of the tissue microenvironment, also called as immunosuppression or immune checkpoint. The high expression of indoleamine 2,3-2,3-dioxygenase in placental tissue can prevent immune rejection to the fetus. The high expression of indoleamine 2,3-dioxygenase in the inflammatory region can prevent excessive immune responses and prevent excessive damage to cell tissue. One of the mechanisms leading to immunosuppression is that the high expression of indoleamine 2,3-dioxygenase causes local L-tryptophan depletion, which is sensed by surrounding lymphocytes through mechanisms such as GCN2, thereby causing cell cycle arrest or apoptosis of CD8+ cytotoxic T cells. Another mechanism leading to immunosuppression is that the high expression of indoleamine 2,3-dioxygenase causes an increase of kynurenine, and after kynurenine formation, it can leave the cell, enter the extracellular matrix, and then enter the nearby lymphocyte, and combine with AHR transcription factors to regulate CD8+ T cells and regulatory Treg cells, where the activity of CD8+ cytotoxic T cells is inhibited, while the number of regulatory Treg cells is increased and activated, thereby causing immunosuppression.

The abnormally high expression of indoleamine 2,3-dioxygenase is present in many different types of tumors including hematologic tumors and solid tumors such as colon cancer, liver cancer, lung cancer, pancreatic cancer, and throat cancer. The abnormally high expression of indoleamine 2,3-dioxygenase is positively correlated with the poor prognosis of tumors. Cancer cell escape immune surveillance is a key step in the canceration and the further development of cancer. The abnormally high expression of indoleamine 2,3-dioxygenase in tumors may be a major mechanism for tumor cell to escape immune surveillance, so the inhibition of the activity of indoleamine 2,3-dioxygenase may activate the suppressed immune system, thereby inhibiting the growth of tumors. Therefore, an indoleamine 2,3-dioxygenase inhibitor as an immune checkpoint inhibitor has aroused great interest in the pharmaceutical industry. There are two kinds of indoleamine 2,3-dioxygenase (IDO), IDO-1 and IDO-2. The main IDO involved in the aforementioned immunosuppression is IDO-1. The role of IDO-2 in immunosuppression is not yet very clear. Tryptophan 2,3-dioxygenase (TDO) is also abnormally highly expressed in many types of tumors, and some tumors also show IDO and TDO double positives, so some people think that the purpose of cancer treatment can be achieved by suppressing TDO immune checkpoints. Because normal liver cells express TDO, it is unclear whether TDO inhibitors affect liver function and normal tryptophan metabolism, but there is no abnormality in a TDO knockout mice model, indicating that TDO inhibitors may not affect liver function and normal tryptophan metabolism. The mechanisms of IDO and TDO leading to immunosuppression are basically the same, so an IDO/TDO bispecific inhibitor also arouses interest in the pharmaceutical industry. The IDO/TDO bispecific inhibitor will be suitable for IDO positive, TDO positive, IDO/TDO double positive patients.

Many metabolites of the kynurenine metabolic pathway of tryptophan are associated with schizophrenia, depression, and neuronal degeneration, and indoleamine 2,3-dioxygenase inhibitors may also be useful in the treatment of these diseases. Kynurenine can be converted to kynurenic acid under the catalysis of kynurenine aminotransferase. Kynurenic acid is an NMDA antagonist, and higher kynurenic acid levels are common in the central nervous system of patients with schizophrenia. Quinolinic acid is neurotoxic and can cause neuronal apoptosis and neurodegeneration. Indoleamine 2,3-dioxygenase is not only involved in the metabolism of tryptophan, but also involved in the metabolism of tryptamine etc. 5-Hydroxytryptamine can be converted to 5-hydroxyindoleacetic acid under the catalysis of indoleamine 2,3-dioxygenase. A decrease of 5-hydroxytryptamine may be one of the factors leading to depression.

Currently, the research and development of indoleamine 2,3-dioxygenase inhibitors, including IDO or TDO inhibitors such as NewLink's Indoximod, NLG-919 (IDO/TDO bispecific), Incyte's Epacadostat (INCB 024360), and BMS, Flexus, Iomet, Iteos, Curadev, etc., are in the early stages. The patent application WO2016041489A1 discloses a series of sulfonimido compounds which have good inhibitory activity against indoleamine 2,3-dioxygenase (IDO), however, the increase in the exposure (AUC) of the best compound 6 disclosed in this patent application is limited, relative to INCB-24360, and the T_{1/2} is very short, which is not conducive to clinical development. Although the T_{1/2} of compound 13 (a prodrug of compound 6) is prolonged, its exposure (AUC) is not as good as INCB-24360. Therefore, further development of compounds with T_{1/2} suitable for clinical administration and high exposure (AUC) has attracted many scientists around the world to make continuous efforts.

### SUMMARY OF THE INVENTION

After a series of studies, the inventors found that N′-hydroxy-N-phenylformamidine derivatives have high inhibitory activity against indoleamine 2,3-dioxygenase (IDO), and have no inhibitory activity against tryptophan 2,3-dioxygenase (TDO). Moreover the derivatives have a very good exposure (AUC) in the PK animal model, and have a T_{1/2} that is very suitable for clinical applications. Such compounds are effective in inhibiting the activity of IDO and can also be used to inhibit immunosuppression in patients. Such compounds can be widely used to treat or prevent cancer or tumor, viral infection, depression, neurodegenerative disease, trauma, age-related cataract, organ transplant rejection or autoimmune diseases, and are expected to be developed into a new generation of immunosuppressive agents.

In one aspect, the present invention provides a N′-hydroxy-N-phenylformamidine derivative having the structure of the following formula (I), a stereoisomer, or a pharmaceutically acceptable salt thereof,

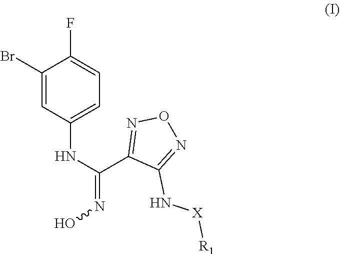

where

[symbol] is a configuration or E configuration, preferably Z configuration;

X is selected from the group consisting of C₁₋₈ alkyl and C₃₋₈ cycloalkyl, optionally substituted by one or more groups selected from the group consisting of deuterium, halogen, hydroxy, thiol, cyano, nitro, azido, C₁₋₈ alkyl, C₂₋₈ alkenyl, C₂₋₈ alkynyl, haloC₁₋₈ alkyl, C₃₋₈ cycloalkyl, 3-8 membered heterocyclyl, 3-8 membered heterocyclyloxy, 3-8 membered heterocyclylthio, C₅₋₁₀ aryl, C₅₋₁₀ aryloxy, C₅₋₁₀ arylthio, 5-10 membered heteroaryl, 5-10-membered heteroaryloxy, 5-10 membered heteroarylthio, —C₀₋₈—S(O)ᵣR₈, —C₀₋₈—O—R₅, —C₀₋₈—C(O)OR₅, —C₀₋₈—C(O)R₆, —C₀₋₈—O—C(O)R₆, —C₀₋₈—NR₇R₈, —C₀₋₈—C(O)NR₇R₈, —N(R₇)—C(O)R₆ and —N(R₇)—C(O)OR₅;

R₁ is selected from the following group consisting of:

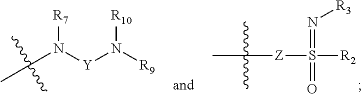

Y is selected from the group consisting of —S(O)₂— and —C(O)—C(O)—;

Z is selected from the group consisting of a bond, O, S and —NR₇—;

R₂ is selected from the group consisting of hydrogen, deuterium, C₁₋₈ alkyl, C₂₋₈ alkenyl, C₂₋₈ alkynyl, C₃₋₈ cycloalkyl, 3-8 membered heterocyclyl, C₅₋₁₀ aryl, 5-10 membered heteroaryl and C₀₋₈ alkylcarbonyl,

optionally substituted by one or more groups selected from the group consisting of halogen, hydroxy, thiol, cyano, nitro, azido, C₁₋₈ alkyl, C₂₋₈ alkenyl, C₂₋₈ alkynyl, haloC₁₋₈ alkyl, C₃₋₈ cycloalkyl, 3-8 membered heterocyclyl, 3-8 membered heterocyclyloxy, 3-8 membered heterocyclylthio, C₅₋₁₀ aryl, C₅₋₁₀ aryloxy, C₅₋₁₀ arylthio, 5-10 membered heteroaryl, 5-10 membered heteroaryloxy, 5-10 membered heteroarylthio, —C₀₋₈—S(O)ᵣR₄, —C₀₋₈—O—R₅, —C₀₋₈—C(O)OR₅, —C₀₋₈—C(O)R₆, —C₀₋₈—O—C(O)R₆, —C₀₋₈—NR₇R₈, —C₀₋₈—C(O)NR₇R₈, —N(R₇)—C(O)R₆ and —N(R₇)—C(O)OR₅;

R₃ is selected from the group consisting of hydrogen, deuterium, hydroxy, amino, C₁₋₈ alkyl, C₂₋₈ alkenyl, C₃₋₈ cycloalkyl, 3-8 membered heterocyclyl, C₅₋₁₀ aryl, 5-10 membered heteroaryl, C₁₋₈ alkoxy, C₃₋₈ cycloalkoxy, 3-8 membered heterocyclyloxy, C₅₋₁₀ aryloxy, 5-10 membered heteroaryloxy, —C₀₋₈—S(O)ᵣR₄, —C₀₋₈—C(O)OR₅, —C₀₋₈—O—C(O)R₆, —C₀₋₈—NR₇R₈, —C₀₋₈—C(O)NR₇R₈, —N(R₇)—C(O)R₆ and —N(R₇)—C(O)OR₅,

optionally substituted by one or more groups selected from the group consisting of halogen, hydroxy, thiol, cyano, nitro, azido, C₁₋₈ alkyl, C₂₋₈ alkenyl, C₂₋₈ alkynyl, haloC₁₋₈ alkyl, C₃₋₈ cycloalkyl, 3-8 membered heterocyclyl, 3-8 membered heterocyclyloxy, 3-8 membered heterocyclylthio, C₅₋₁₀ aryl, C₅₋₁₀ aryloxy, C₅₋₁₀ arylthio, 5-10 membered heteroaryl, 5-10 membered heteroaryloxy, 5-10 membered heteroarylthio, —C₀₋₈—S(O)ᵣR₄, —C₀₋₈—O—R₅, —C₀₋₈—C(O)OR₅, —C₀₋₈—C(O)R₆, —C₀₋₈—O—C₀₋₈—NR₇R₈, —C₀₋₈—C(O)NR₇R₈, —N(R₇)—C(O)R₆ and —N(R₇)—C(O)OR₅;

R₄ is selected from the group consisting of hydrogen, deuterium, C₁₋₈ alkyl, C₂₋₈ alkenyl, C₃₋₈ cycloalkyl, haloC₁₋₈ alkyl, phenyl, p-methylphenyl, amino, mono C₁₋₈ alkylamino, di C₁₋₈ alkylamino and C₁₋₈ alkanoylamino;

R₅ is selected from the group consisting of hydrogen, deuterium, C₁₋₈ alkyl, C₃₋₈ cycloalkyl, haloC₁₋₈ alkyl, and hydroxyC₁₋₈ alkyl;

R₆ is selected from the group consisting of hydrogen, deuterium, C₁₋₈ alkyl, C₁₋₈ alkoxy, C₃₋₈ cycloalkyl, C₃₋₈ cycloalkoxy, haloC₁₋₈ alkyl, haloC₁₋₈ alkoxy, hydroxyC₁₋₈ alkyl and hydroxy C₁₋₈ alkoxy;

R₇, R₈, R₉, and R₁₀ are each independently selected from the group consisting of hydrogen, deuterium, hydroxy, C₁₋₈ alkyl, hydroxyC₁₋₈ alkyl, C₁₋₈ alkoxy, C₂₋₈ alkenyl, C₂₋₈ alkynyl, C₃₋₈ cycloalkyl, 3-8 membered heterocyclyl, C₅₋₁₀ aryl, 5-10 membered heteroaryl and C₁₋₈ alkanoyl, or R₇ and R₈, R₉ and R₁₀ together with the nitrogen atom to which they are attached form a 3-8 membered heterocycloalkyl,

optionally substituted by one or more groups selected from the group consisting of halogen, hydroxy, thiol, cyano, nitro, acetamido, azido, sulfonyl, methylsulfonyl, C₁₋₈ alkyl, trifluoromethyl, C₂₋₈ alkenyl, C₂₋₈ alkynyl, C₃₋₈ cycloalkyl, 3-8 membered heterocyclyl, C₁₋₈ alkoxy, C₁₋₈ alkoxycarbonyl, C₁₋₈ alkylcarbonyl, C₁₋₈ alkylcarbonyloxy, 3-8 membered heterocyclyloxy, 3-8 membered heterocyclylthio, C₅₋₁₀ aryl, C₅₋₁₀ aryloxy, C₅₋₁₀ arylthio, 5-10 membered heteroaryl, 5-10 membered heteroaryloxy, 5-10 membered heteroarylthio, amino, mono C₁₋₈ alkylamino, and di C₁₋₈ alkylamino; and

r is 0-2.

In a further preferred embodiment, the (Z)—N′-hydroxy-N-phenylformamidine derivative, the stereoisomer or the pharmaceutically acceptable salt thereof, is the compound of formula (II):

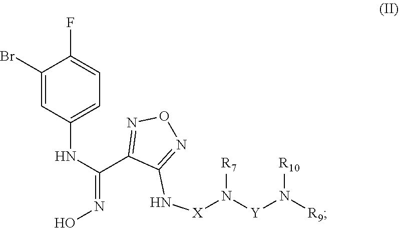

wherein:

X is selected from the group consisting of C₁₋₆ alkyl and C₃₋₈ cycloalkyl, optionally substituted by one or more groups selected from the group consisting of deuterium, halogen, hydroxy, thiol, cyano, nitro, azido, C₁₋₈ alkyl, haloC₁₋₈ alkyl and C₃₋₈ cycloalkyl; R₇, R₉, and R₁₀ are each independently selected from the group consisting of hydrogen, deuterium, hydroxy, C₁₋₈ alkyl, hydroxyC₁₋₈ alkyl, C₁₋₈ alkoxy, C₂₋₈ alkenyl, C₂₋₈ alkynyl, C₃₋₈ cycloalkyl, 3-8 membered heterocyclyl, C₅₋₁₀ aryl, C₅₋₁₀ aryl substituted by C₁₋₈ alkyl, 5-10 membered heteroaryl, C₁₋₈ alkanoyl and —C₀₋₈—C(O)OR₅, or R₉ and R₁₀ together with the nitrogen atom to which they are attached form a 5-6 membered heterocycloalkyl,

optionally substituted by one or more groups selected from the group consisting of halogen, hydroxy, thiol, cyano, nitro, acetamido, azido, sulfonyl, methylsulfonyl, C₁₋₈ alkyl, trifluoromethyl, C₃₋₈ cycloalkyl, 3-8 membered heterocyclyl, C₁₋₈ alkoxy, C₁₋₈ alkoxycarbonyl, C₁₋₈ alkylcarbonyl, C₁₋₈ alkylcarbonyloxy, 3-8 membered heterocyclyloxy, 3-8 membered heterocyclylthio, C₅₋₁₀ aryl, C₅₋₁₀ aryloxy, C₅₋₁₀ arylthio, 5-10 membered heteroaryl, 5-10 membered heteroaryloxy, 5-10 membered heteroarylthio, amino, mono C₁₋₈ alkylamino, and di C₁₋₈ alkylamino.

In a still preferred embodiment, the (Z)—N′-hydroxy-N-phenylcarboxamidine derivative, the stereoisomer or the pharmaceutically acceptable salt thereof, is selected from the group consisting of a compound of formula (IIA) and a compound of (IIB):

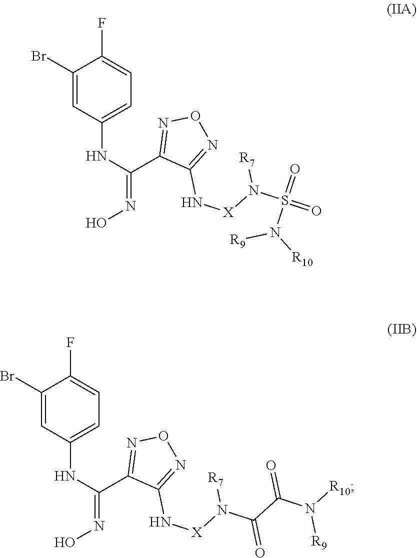

wherein:

X is selected from the group consisting of ethyl, cyclobutyl and cyclohexyl, optionally substituted by one or more groups selected from the group consisting of deuterium, halogen, hydroxy, thiol, cyano, nitro, trifluoromethyl, C₁₋₈ alkyl and C₃₋₈ cycloalkyl; and

R₇, R₉, and R₁₀ are each independently selected from the group consisting of hydrogen, deuterium, hydroxy, C₁₋₈ alkyl, hydroxyC₁₋₈ alkyl, C₁₋₈ alkoxy, C₃₋₈ cycloalkyl, 3-8 membered heterocyclyl, C₅₋₁₀ aryl, C₅₋₁₀ aryl substituted by C₁₋₈ alkyl, 5-10 membered heteroaryl, C₁₋₈ alkanoyl and —C₀₋₈—C(O)OR₅, or R₉ and R₁₀ together with the nitrogen atom to which they are attached form a 5-6 membered heterocycloalkyl,

In the most preferred embodiment, the (Z)—N′-hydroxy-N-phenylcarboxamidine derivative, the stereoisomer or the pharmaceutically acceptable salt thereof, is selected from the group consisting of:

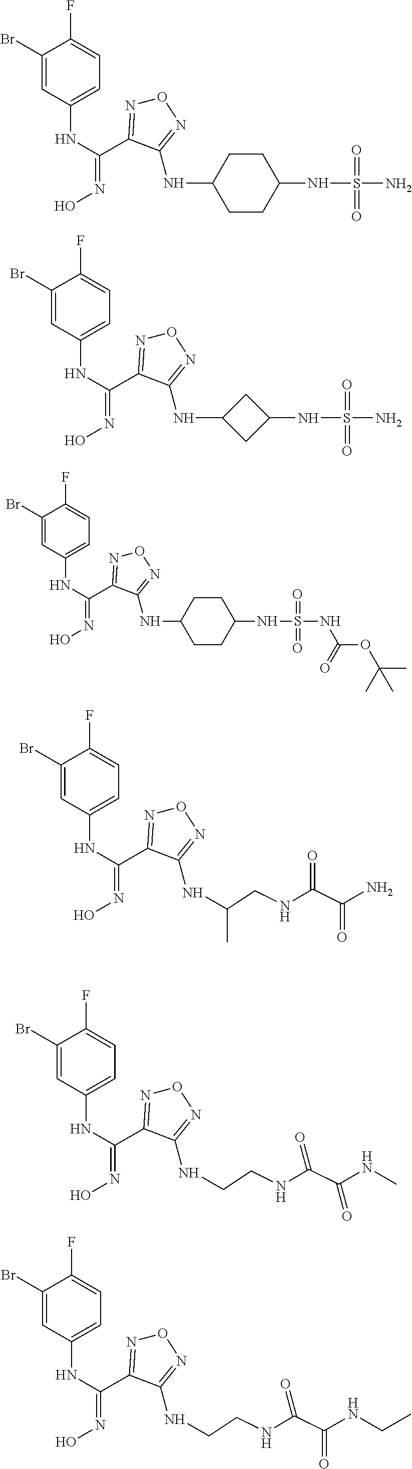

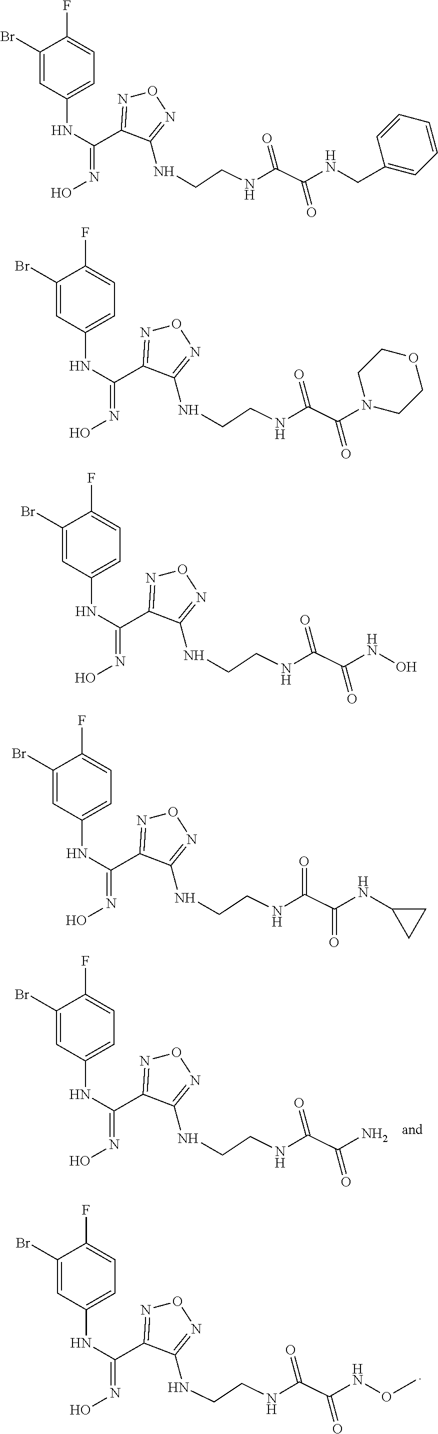

In a further preferred embodiment, the (Z)—N′-hydroxy-N-phenylcarboxamidine derivative, the stereoisomer or the pharmaceutically acceptable salt thereof, is a compound of formula (III):

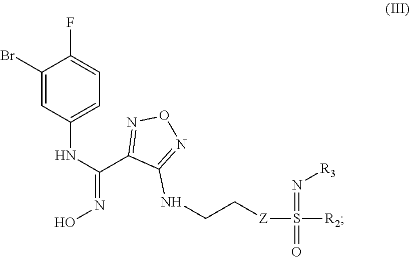

Z is selected from the group consisting of a bond and —NR₇—;

R₂ is selected from the group consisting of hydrogen, deuterium, and C₁₋₈ alkyl;

R₃ is selected from the group consisting of deuterium, hydroxy, amino, C₁₋₈ alkyl, C₃₋₈ cycloalkyl, 3-8 membered heterocyclyl, C₅₋₁₀ aryl, 5-10 membered heteroaryl, C₁₋₈ alkoxy, C₃₋₈ cycloalkoxy, 3-8 membered heterocyclyloxy, C₅₋₁₀ aryloxy, 5-10 membered heteroaryloxy, —C₀₋₈—S(O)ᵣR₄, —C₀₋₈—C(O)OR₅ and —C₀₋₈—OC(O)R₆;

R₄ is selected from the group consisting of hydrogen, deuterium, C₁₋₈ alkyl, C₂₋₈ alkenyl, C₃₋₈ cycloalkyl, haloC₁₋₈ alkyl, phenyl, p-methylphenyl, amino, mono C₁₋₈ alkylamino, di C₁₋₈ alkylamino and C₁₋₈ alkanoylamino;

R₅ is selected from the group consisting of hydrogen, deuterium, C₁₋₈ alkyl, C₃₋₈ cycloalkyl, haloC₁₋₈ alkyl, and hydroxyC₁₋₈ alkyl;

R₆ is selected from the group consisting of hydrogen, deuterium, C₁₋₈ alkyl, C₁₋₈ alkoxy, C₃₋₈ cycloalkyl, C₃₋₈ cycloalkoxy, haloC₁₋₈ alkyl, haloC₁₋₈ alkoxy, hydroxyC₁₋₈ alkyl and hydroxyC₁₋₈ alkoxy; and

r is 0, 1 or 2.

In a still further preferred embodiment, the (Z)—N′-hydroxy-N-phenylcarboxamidine derivative, the stereoisomer or the pharmaceutically acceptable salt thereof, is a compound of formula (III) having the two following structures:

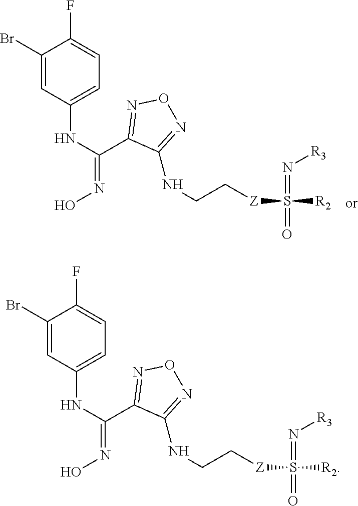

In a still further preferred embodiment, in the compound of formula (I), the stereoisomers or pharmaceutically acceptable salts thereof,

Z is selected from the group consisting of a bond and —NR₇—;

R₂ is selected from the group consisting of methyl, ethyl, propyl, isopropyl, cyclopropyl, cyclobutyl, cyclopentyl, cyclohexyl and phenyl;

R₃ is selected from the group consisting of hydroxy, amino, C₁₋₈ alkyl, and —C₀₋₈—S(O)ᵣR₄;

R₄ is selected from the group consisting of hydrogen, deuterium, C₁₋₈ alkyl, C₂₋₈ alkenyl, C₃₋₈ cycloalkyl, haloC₁₋₈ alkyl, phenyl, p-methylphenyl, amino, mono C₁₋₈ alkylamino, di C₁₋₈ alkylamino, and C₁₋₈ alkanoylamino; and

r is 0-2.

In the most preferred embodiment, the (Z)—N′-hydroxy-N-phenylformamidine derivative, the stereoisomer or the pharmaceutically acceptable salt thereof, is selected from the group consisting of:

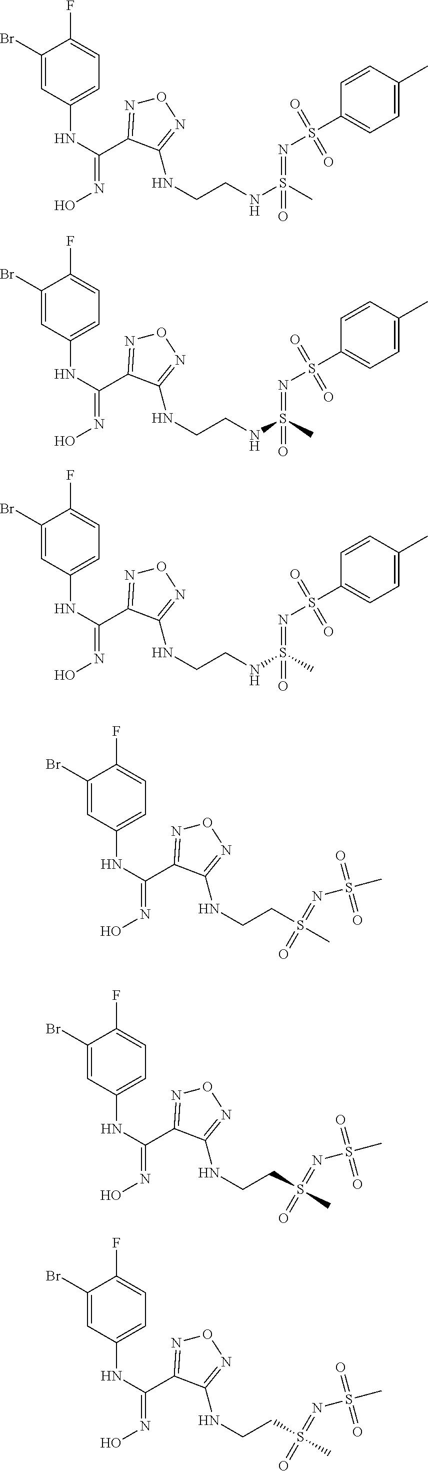

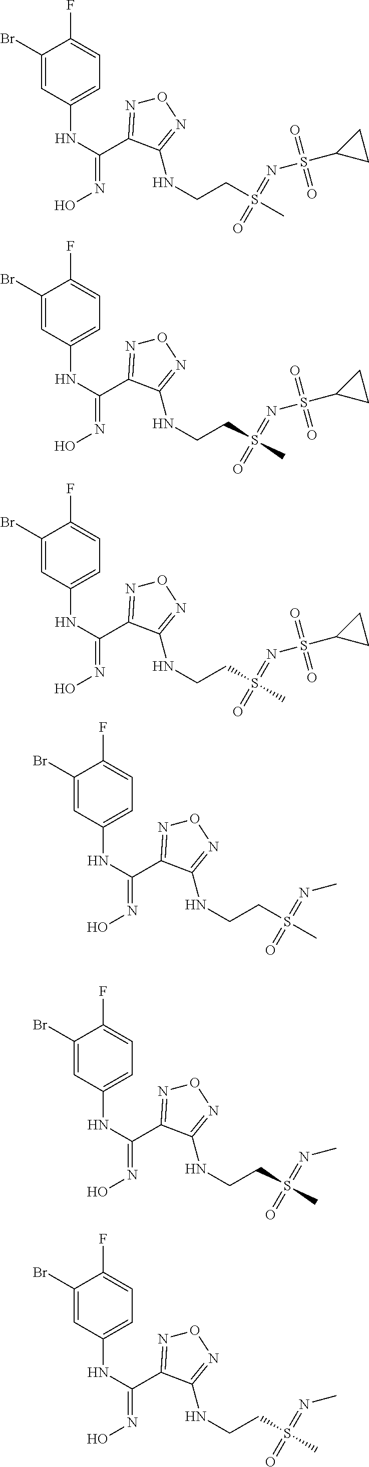

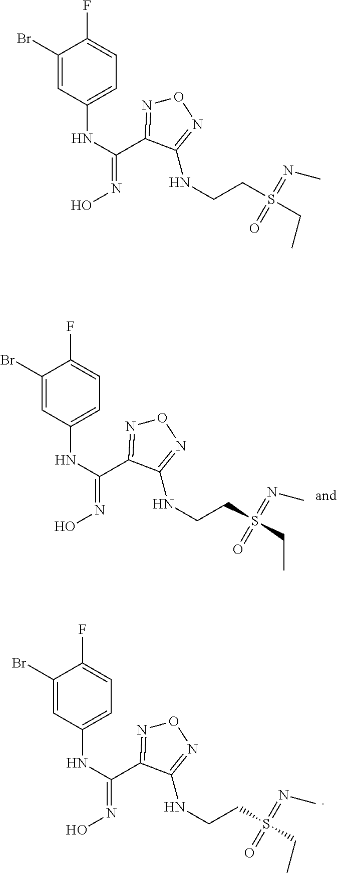 [SMILES: CCS(=O)(CCNc1nonc1/C(=N/O)Nc1ccc(F)c(Br)c1)=NC ; CC[S@@](=O)(CCNc1nonc1/C(=N/O)Nc1ccc(F)c(Br)c1)=NC ; CC[S@](=O)(CCNc1nonc1/C(=N/O)Nc1ccc(F)c(Br)c1)=NC]

The present invention also relates to an intermediate or preparing the compound of formula (III), the stereoisomer or the pharmaceutically acceptable salt thereof, characterized in that the intermediate is a compound of formula (IV), a stereoisomer or a pharmaceutically acceptable salt thereof:

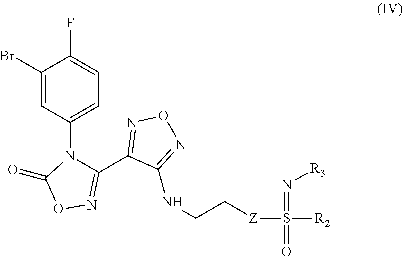

wherein:

Z, R₂, and R₃ are as defined in formula (III).

The present invention also relates to a process for preparing the compound of formula (I), the stereoisomer or the pharmaceutically acceptable salt thereof, characterized in that the process comprises the following step of:

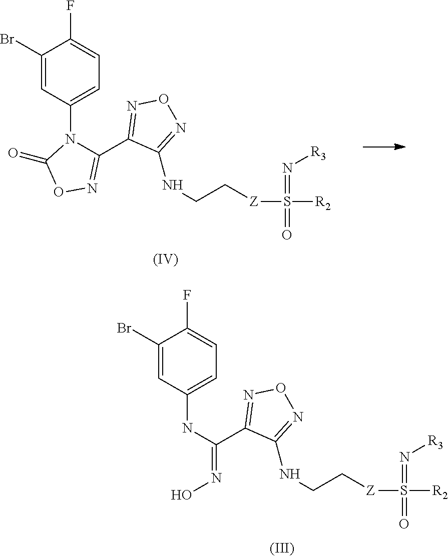

opening a ring of a compound of formula (IV) under an alkaline condition to obtain a compound of formula (III);

wherein:

Z, R₂, and R₃ are as described in formula (III). In another aspect, the present invention provides a process for preparing the aforementioned N′-hydroxy-N-phenylformamidine derivative, the stereoisomer or the pharmaceutically acceptable salt thereof, comprising the following preparation step of:

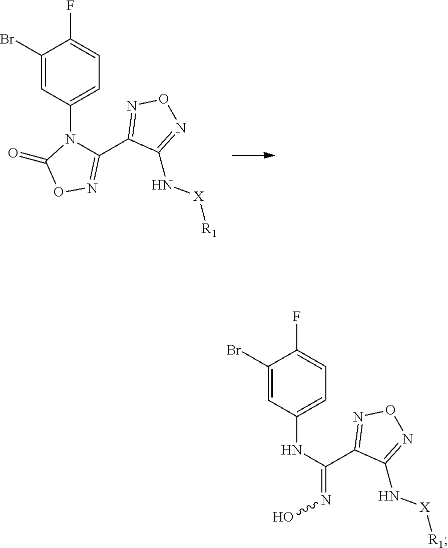

wherein:

X, and R₁ are as defined in the compound of formula (I).

In another aspect, the present invention provides a pharmaceutical composition comprising a therapeutically effective amount of the aforementioned compound of formula (I), the stereoisomer or the pharmaceutically acceptable salt thereof, and a pharmaceutically acceptable carrier.

In another aspect, the present invention relates to a use of the aforementioned compound of formula (I), the stereoisomer or the pharmaceutically acceptable salt thereof, or the aforementioned pharmaceutical composition in the preparation of a medicament for inhibiting the activity of indoleamine 2,3-dioxygenase or for inhibiting immunosuppression in patients.

In another aspect, the present invention relates to a use of the aforementioned compound of formula (I), the stereoisomer or the pharmaceutically acceptable salt thereof, or the aforementioned pharmaceutical composition in the preparation of a medicament for treating or preventing cancer or tumor, viral infection, depression, neurodegenerative disorder, trauma, age-related cataract, organ transplant rejection or autoimmune disease in patients; wherein the cancer or tumor is preferably selected from the group consisting of lung cancer, bone cancer, gastric cancer, pancreatic cancer, skin cancer, head and neck cancer, uterine cancer, ovarian cancer, testicular cancer, uterine cancer, fallopian tube cancer, endometrial cancer, cervical cancer, vaginal cancer, vulvar cancer, rectal cancer, colon cancer, anal cancer, breast cancer, esophageal cancer, small intestine cancer, endocrine system cancer, thyroid cancer, parathyroid cancer, adrenal cancer, urethral cancer, penile cancer, prostate cancer, pancreatic cancer, brain cancer, testicular cancer, lymph cancer, transitional cell cancer, bladder cancer, kidney or ureter cancer, renal cell carcinoma, renal pelvis cancer, Hodgkin's disease, non-non-Hodgkin's lymphoma, soft tissue sarcoma, solid tumor in children, lymphocytic lymphoma, central nervous system (CNS) tumor, primary central nervous system lymphoma, tumor angiogenesis, spinal tumor, brainstem glioma, pituitary adenoma, melanoma, Kaposi's sarcoma, epidermoid carcinoma, squamous cell carcinoma, T cell lymphoma, chronic or acute leukemia, and a combination of the aforementioned cancers.

In a further preferred embodiment, the use refers to that a therapeutically effective amount of the aforementioned compound of formula (I), the stereoisomer or the pharmaceutically acceptable salt thereof, or the aforementioned pharmaceutical composition is combined with an anti-CTLA-4 antibody, an anti-PD-1 antibody, an anti-PD-L1 antibody, a antiviral agent, a chemotherapeutic agent, an immunosuppressant, radiation, an anti-tumor vaccine, an antiviral vaccine, a cytokine therapy or a tyrosine kinase inhibitor; the cytokine is preferably IL-2, IL-3, IL-4, or IL-5, and the chemotherapeutic agent is preferably a cytotoxic agent, and the anti-PD-1 antibody is preferably a Keytruda antibody.

In another aspect, the invention provides a method of modulating the activity of indoleamine 2,3-dioxygenase, comprising contacting a therapeutically effective amount of the aforementioned compound of formula (I), the stereoisomer or the pharmaceutically acceptable salt, or the aforementioned pharmaceutical composition with indoleamine 2,3-dioxygenase; preferably, the modulation is an inhibitory effect.

In another aspect, the present invention provides a method for inhibiting immunosuppression in patients, comprising administering a therapeutically effective amount of the aforementioned compound of Formula (I), the stereoisomer or the pharmaceutically acceptable salt thereof, or the aforementioned pharmaceutical composition to the patients.

In another aspect, the present invention relates to a method for treating cancer, comprising administering to a patient a therapeutically effective amount of the compound of formula (I) of the present invention or the tautomer, mesomer, racemate, enantiomer, diastereomer thereof, or a mixture thereof, or the pharmaceutically acceptable salt thereof. The method shows outstanding efficacy and fewer side effects, wherein the cancer or tumor is selected from the group consisting of lung cancer, bone cancer, gastric cancer, pancreatic cancer, skin cancer, head and neck cancer, uterine cancer, ovarian cancer, testicular cancer, uterine cancer, fallopian tube cancer, endometrial cancer, cervical cancer, vaginal cancer, vulvar cancer, rectal cancer, colon cancer, anal cancer, breast cancer, esophageal cancer, small intestine cancer, endocrine system cancer, thyroid cancer, parathyroid cancer, adrenal cancer, urethral cancer, penile cancer, prostate cancer, pancreatic cancer, brain cancer, testicular cancer, lymph cancer, transitional cell cancer, bladder cancer, kidney or ureter cancer, renal cell renal pelvis cancer, Hodgkin's disease, non-Hodgkin's lymphoma, soft tissue sarcoma, solid tumor in children, lymphocytic lymphoma, central nervous system (CNS) tumor, primary central nervous system lymphoma, tumor angiogenesis, spinal tumor, brainstem glioma, pituitary adenoma, melanoma, Kaposi's sarcoma, epidermoid carcinoma, squamous cell carcinoma, T cell lymphoma, chronic or acute leukemia, and a combination of the aforementioned cancers.

### DESCRIPTION OF THE DRAWINGS

FIG. 1 shows the detection spectrum of the compound of Example 15; the abscissa represents retention time (unit: min) and the ordinate represents response value (unit: mV);

FIG. 2 shows the detection spectrum of optical isomer \textcircled{1}; the abscissa represents retention time (unit: min) and the ordinate represents response value (unit: mV);

FIG. 3 shows the detection spectrum of optical isomer \textcircled{2}; the abscissa represents the retention time (unit: min) and the ordinate represents response value (unit: mV).

### DETAILED DESCRIPTION OF THE INVENTION

Detailed description: unless otherwise stated, the following terms which are used in the description and the claims have the following meanings.

“C₁₋₈ alkyl” refers to a straight chain or branched chain alkyl group having 1 to 8 carbon atoms, “alkyl” refers to a saturated aliphatic hydrocarbon group, C₀₋₈ refers to carbon-free and C₁₋₈ alkyl group, preferably includes a straight chain alkyl group having 1 to 6 carbon atoms, more preferably includes a straight chain alkyl group having 1 to 4 carbon atoms, for example, methyl, ethyl, n-propyl, isopropyl, n-butyl, isobutyl, t-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl and various branched chain isomers thereof and the like.

“Cycloalkyl” refers to a saturated or partially unsaturated monocyclic or polycyclic hydrocarbon substituent, “C₃₋₈ cycloalkyl” refers to a cycloalkyl group having 3 to 8 carbon atoms, “5-10 membered cycloalkyl” refers to a cycloalkyl group having 5 to 10 carbon atoms, for example: non-limiting examples of monocyclic cycloalkyl include cyclopropyl, cyclobutyl, cyclopentyl, cyclopentenyl, cyclohexyl, cyclohexenyl, cyclohexadienyl, cycloheptyl, cycloheptatrienyl, cyclooctyl and the like, preferably cyclopropyl, cyclobutyl or cyclohexyl; polycyclic cycloalkyl includes a cycloalkyl having a spiro ring, fused ring and bridged ring.

“Heterocyclyl” refers to a saturated or partially unsaturated monocyclic or polycyclic hydrocarbon substituent, wherein one or more ring atoms are heteroatoms selected from the group consisting of nitrogen, oxygen, and S(O)r (wherein r is an integer of 0, 1, or 2), but the cyclic part does not include —O—O—, —O—S— or —S—S—, and the remaining ring atoms are carbon. “5-10 membered heterocyclyl” refers to a heterocyclyl group having 5 to 10 ring atoms, and “3-8 membered heterocyclyl” refers to a heterocyclyl group having 3 to 8 ring atoms, and 5-6 membered heterocyclyl is preferred.

Non-limiting examples of monocyclic heterocyclyl include pyrrolidinyl, piperidinyl, piperazinyl, morpholinyl, thiomorpholinyl, homopiperazinyl and the like, preferably morphine.

Polycyclic heterocyclic includes a heterocyclyl having a spiro ring, fused ring or bridged ring.

“Aryl” refers to an all-carbon monocycle or fused polycycle (i.e., a ring in the system shares an adjacent pair of carbon atoms with another ring in the system) having a conjugated it electron system. “C₅₋₁₀ aryl” refers to an all-carbon aryl group having 5-10 carbons, and “5-10 membered aryl” refers to an all-carbon aryl group having 5-10 carbons, for example, phenyl and naphthalene.

“Heteroaryl” refers to a heteroaromatic system having 1 to 4 heteroatoms, wherein the heteroatoms include nitrogen, oxygen, and S(O)r (wherein r is an integer of 0, 1, or 2). 5-7 membered heteroaryl refers to a heteroaromatic system having 5-7 ring atoms, and 5-10 membered heteroaryl refers to a heteroaromatic system having 5-10 ring atoms, for example, furyl, thienyl, pyridyl, pyrrolyl, N-alkylpyrrolyl, pyrimidyl, pyrazinyl, imidazolyl, tetrazolyl, and the like.

“Alkenyl” refers to an alkyl group as defined above that has at least two carbon atoms and at least one carbon-carbon double bond, and C₂₋₈ alkenyl refers to a straight chain or branched chain alkenyl group having 2-8 carbons, for example, vinyl, 1-propenyl, 2-propenyl, 1-, 2-, or 3-butenyl, and the like.

“Alkynyl” refers to an alkyl group as defined above that has at least two carbon atoms and at least one carbon-carbon triple bond, and C₂₋₈ alkynyl refers to a straight chain or branched alkynyl group having 2-8 carbons, for example, ethynyl, 1-propynyl, 2-propynyl, 1-, 2-, or 3-butynyl, and the like.

“Alkoxy” refers to an —O-(alkyl), wherein the alkyl is as defined above. C₁₋₈ alkoxy refers to an alkoxy having 1-8 carbons, and non-limiting examples include methoxy, ethoxy, propoxy, butoxy and the like.

“haloC₁₋₈ alkyl” refers to a C₁₋₈ alkyl group, wherein hydrogens in the alkyl are substituted by fluorine, chlorine, bromine and iodine atoms, for example, difluoromethyl, dichloromethyl, dibromomethyl, trifluoromethyl, trichloromethyl, tribromomethyl and the like.

“haloC₁₋₈ alkoxy” refers to a C₁₋₈ alkoxy group, wherein hydrogens in the alkyl are substituted by fluorine, chlorine, bromine and iodine atoms, for example, difluoromethoxy, dichloromethoxy, dibromomethoxy, trifluoromethoxy, trichloromethoxy, tribromomethoxy and the like.

“Halogen” refers to fluorine, chlorine, bromine, or iodine.

“Optional” or “optionally” means that the subsequently described event or the circumstance can, but need not occur. Its meaning includes the instances in which the event or the circumstance does or does not occur. For example, “heterocyclyl optionally substituted by alkyl” means that the alkyl group can be, but need not be present. Its meaning includes the instances in which heterocyclyl is substituted or unsubstituted by alkyl.

“Substituted” means that one or more hydrogen atoms, preferably up to 5, and more preferably 1 to 3 hydrogen atoms in the group are each independently substituted by the corresponding number of the substituents. Obviously, the substituents are only positioned at their possible chemical positions, and the possible or impossible substitutions can be determined (through experiments or theory) by those skilled in the art without paying excessive efforts. For example, the combination of amino or hydroxy having free hydrogen and carbon atoms having unsaturated bonds (such as olefinic) may be unstable.

“Pharmaceutical composition” refers to a mixture comprising one or more of the compounds described herein or the physiological/pharmaceutical salts or prodrugs thereof and other chemical components, such as physiological/pharmaceutical carriers and excipients. The purpose of the pharmaceutical composition is to facilitate administration of a compound to an organism, which will help with absorption of the active ingredient, thereby realizing biological activity.

“Stereoisomerism” includes geometric isomerism (cis-trans isomerism), optical isomerism, and conformational isomerism.

The following examples serve to illustrate the present invention in detail and more completely, but these examples should not be considered as limiting the scope of the present invention, and the present invention is not limited to the examples.

The structures of compounds in the present invention were identified by nuclear magnetic resonance (NMR) and/or liquid chromatography-mass spectrometry (LC-MS). The chemical shift of NMR is given in 10⁻⁶ (ppm). NMR was determined by a Bruker AVANCE-400 machine, the solvents for determination are deuterated methanol (CD₃OD) and deuterated chloroform (CDCl₃), and the internal standard is tetramethylsilane (TMS).

Liquid chromatography-mass spectrometry (LC-MS) was determined by an Agilent 1200 Infinity Series mass spectrometer. HPLC was determined on an Agilent 1200DAD high pressure liquid chromatographic instrument (Sunfire C18 150×4.6 mm chromatographic column) and a Waters 2695-2996 high pressure liquid chromatographic instrument (Gimini C18 150×4.6 mm chromatographic column).

For thin-layer silica gel chromatography (TLC), Yantai Huanghai HSGF254 or Qingdao GF254 silica gel plate was used. The dimension of the plates used in TLC was 0.15 mm to 0.2 mm, and the dimension of the plates used in product purification was 0.4 mm to 0.5 mm. Column chromatography generally used Yantai Huanghai 200 to 300 mesh silica gel as carrier.

The starting materials used in the examples of the present invention are known and commercially available, or can be synthesized by adopting or according to known methods in the art.

Unless otherwise stated, all reactions of the present invention are carried out under continuous magnetic stirring in a dry nitrogen or argon atmosphere, and the solvent is dry.

An argon atmosphere or nitrogen atmosphere means that the reaction flask is connected to an about 1 L argon or nitrogen balloon. A hydrogen atmosphere means that the reaction flask is connected to an about 1 L hydrogen balloon.

Unless otherwise specified, the solution in the examples refers to an aqueous solution. The reaction temperature is room temperature. Room temperature is the most suitable reaction temperature and is 20° C. to 30° C. The reaction process was monitored by thin layer chromatography (TLC) or the liquid chromatography-mass spectrometry (LC-MS) in the examples. The developing solvent systems included: dichloromethane and methanol system, n-hexane and ethyl acetate system, petroleum ether and ethyl acetate system, acetone. The ratio of the volume of the solvent was adjusted according to the polarity of the compounds. The eluent systems for column chromatography included: A: dichloromethane and methanol system, B: n-hexane and ethyl acetate system, C: dichloromethane and ethyl acetate system, D: ethyl acetate and methanol. The ratio of the volume of the solvent was adjusted according to the polarity of the compounds, and sometimes a little ammonia or acetic acid was added.

### Synthesis of Intermediates

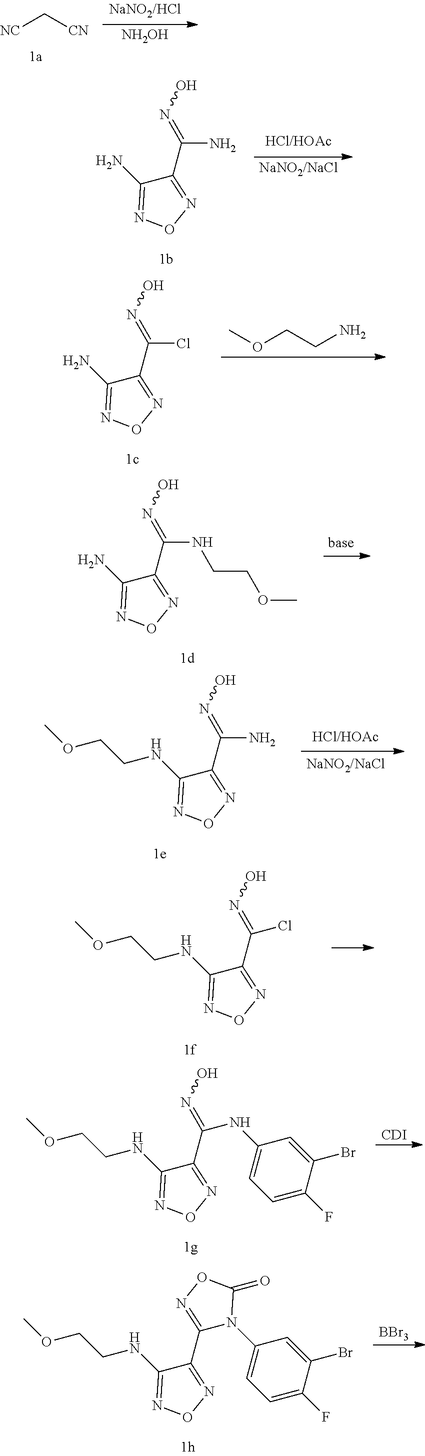

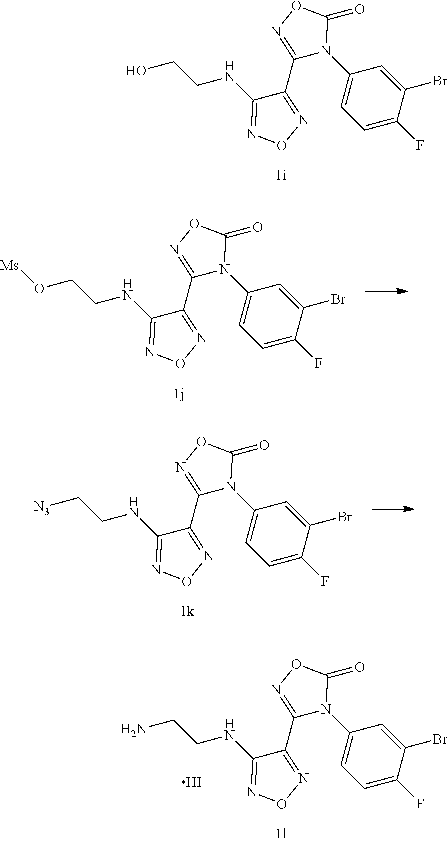

### Step 1: 4-amino-N′-hydroxy-1,2,5-oxadiazole-3-carboximidamide 1b

Malonic cyanide (20 g, 303 mmol) was dissolved in 350 mL of water, and the solution was heated to 45° C. for 5 minutes. Sodium nitrite (23 g, 333.3 mmol) was added under an ice bath. After the temperature rose to 10° C., 6N HCl (3.4 mL) was added. The reaction mixture was stirred at 16-18° C. for 1.5 hours after the temperature rised to 16° C. Then the mixture was cooled to 13° C., and 50% aqueous hydroxylamine solution (61.7 g, 909 mmol) was added in one portion. Then the temperature rose sharply to 27° C., and the mixture was stirred at this temperature for 1 hour, and then heated to reflux for 2 hours. After cooling to room temperature, the reaction mixture was stirred overnight. 6N HCl (49 mL) was added under an ice bath to adjust the pH to 7. The reaction mixture was continuely stirred under an ice bath. A solid was precipitated and filtered. The filter cake was washed with water and dried to obtain the compound 4-amino-N′-hydroxy-1,2,5-oxadiazole-3-carboximidamide 1b (40 g, 92%).

MS m/z (ESI): 143.9.

¹³C NMR (400 MHz, CD₃OD, ppm): δ 154.5, 144.4, 139.7.

### Step 2: 4-amino-N-hydroxy-1,2,5-oxadiazole-3-carbimidoyl chloride 1c

The compound 4-amino-N′-hydroxy-1,2,5-oxadiazole-3-carboximidamide (8.4 g, 59 mmol) was dissolved in a mixture of water (100 mL) and acetic acid (60 mL). 6N HCl (29 mL) was added. The mixture was heated until the solute was completely dissolved. Then, NaCl (10.36 g, 59.5 mmol) was added, followed by the addition of an aqueous sodium nitrite (3.99 g, 5.78 mmol) solution (14 mL) under an ice bath. The reaction mixture was stirred at 0° C. for 1.5 hours, and then warmed up to room temperature. A solid was precipitated and filtered. The filter cake was washed with water and dried to obtain the compound 4-amino-N-hydroxy-1,2,5-oxadiazole-3-4-amino-N-hydroxy-1,2,5-oxadiazole-3-carbimidoyl chloride 1c (4 g, 42%).

MS m/z (ESI): 162.9.

¹³CNMR (400 MHz, CD₃OD, ppm): δ 154.3, 141.9, 127.0.

### Step 3: 4-amino-N′-hydroxy-N-(2-methoxyethyl)-1,2,5-oxadiazole-3-carboximidamide 1d

The compound 4-amino-N-hydroxy-1,2,5-oxadiazole-3-carbimidoyl chloride (4.0 g, 24.7 mmol) was dissolved in ethyl acetate (40 mL). 2-methoxyethane-1-amine (2.29 mL, 25.9 mmol) was added under an ice bath, and the mixture was stirred for 5 minutes. Then triethylamine (5.16 mL, 37.05 mmol) was added. The reaction mixture was stirred for 2 hours until the reaction was completed. The mixture was washed with water and saturated brine. The organic phase was dried over anhydrous sodium sulfate and concentrated in vacuo to obtain the compound 4-amino-N′-hydroxy-N-(2-methoxyethyl)-1,2,5-oxadiazole-3-carboximidamide 1d (4.5 g, 92%).

MS m/z (ESI): 202.1.

¹H NMR (400 MHz, DMSO, ppm): δ 10.67 (s, 1H), 6.28 (s, 2H), 6.14 (s, 1H), 3.56 (m, 2H), 3.44 (m, 2H), 3.28 (s, 3H).

### Step 4: N¹-hydroxy-4-((2-methoxyethyl)amino)-1,2,5-oxadiazole-3-carboximidamide 1e

The compound 4-amino-N′-hydroxy-N-(2-methoxyethyl)-1,2,5-oxadiazole-3-carboximidamide (4.5 g, 22.3 mmol) was dissolved in water (40 mL). After potassium hydroxide (4.15 g, 74.1 mmol) was added, the mixture was refluxed for 48 hours until the reaction was completed. The reaction mixture was extracted with ethyl acetate. The organic phase was washed with water and saturated brine, dried over anhydrous sodium sulfate and concentrated in vacuo to obtain the compound N′-hydroxy-4-((2-methoxyethyl)amino)-1,2,5-oxadiazole-3-carboximidamide 1e (2.8 g, 62%).

MS m/z (ESI): 202.1.

¹H NMR (400 MHz, DMSO-d₆, ppm): δ 10.53 (s, 1H), 6.22 (s, 2H), 6.15 (s, 1H), 3.56 (m, 2H), 3.50 (m, 2H), 3.37 (s, 3H).

### Step 5: N-hydroxy-4-((2-methoxyethyl)amino)-1,2,5-oxadiazole-3-carbimidoyl chloride 1f

The compound N′-hydroxy-4-((2-methoxyethyl)amino)-1,2,5-oxadiazol-3-N′-hydroxy-4-((2-methoxyethyl)amino)-1,2,5-oxadiazol-3-carboximidamide (2.8 g, 13.93 mmol) was dissolved in 6N HCl (14 mL). After the solution was clear, sodium chloride solution (2.2 g, 41.79 mmol) was added. Then water (14 mL) and ethyl acetate (14 mL) were added. Sodium nitrite (1.0 g, 13.3 mmol) was added dropwise under an ice bath. The reaction mixture was stirred under an ice bath for 2 hours, and then stirred at room temperature overnight until the reaction was completed. The reaction mixture was extracted with ethyl acetate. The organic phase was washed with water and saturated brine, dried over anhydrous sodium sulfate and concentrated in vacuo to obtain a solid. The solid was washed with ethyl acetate: petroleum ether to obtain the compound N-hydroxy-4-((2-methoxyethyl)amino)-1,2,5-oxadiazole-3-N-hydroxy-4-((2-methoxyethyl)amino)-1,2,5-oxadiazole-3-carbimidoyl chloride if (2.2 g, 72%).

MS m/z (ESI): 221.1.

¹HNMR (400 MHz, DMSO-d₆, ppm): δ 13.47 (s, 1H), 6.22 (s, 2H), 5.99 (s, 1H), 3.43 (m, 2H), 3.53 (m, 2H), 3.28 (s, 3H).

### Step 6: N-(3-bromo-4-fluorophenyl)-N′-hydroxy-4-((2-methoxyethyl)amino)-1,2,5-oxadiazole-3-carboximidamide 1g

The compound N-hydroxy-4-((2-methoxyethyl)amino)-1,2,5-oxadiazole-3-carbimidoyl chloride (2.2 g, 10 mmol) was added in water (14 mL), and the mixture was heated to 60° C. Then 3-bromo-4-fluoroaniline (2.06 g, 11 mmol) was added, and the mixture was stirred for 10 minutes. Sodium bicarbonate (1.26 g, 15 mmol) was added, and the mixture was stirred at 60° C. for 30 minutes until the reaction was completed. The reaction mixture was extracted with ethyl acetate. The organic phase was washed with water and saturated brine, dried over anhydrous sodium sulfate and concentrated in vacuo to obtain the compound 1g (3.9 g, 105%).

MS m/z (ESI): 374.0.

¹H NMR (400 MHz, DMSO-d₆, ppm): δ 11.54 (s, 1H), 8.86 (s, 2H), 7.10 (m, 1H), 6.81 (m, 1H), 6.15 (m, 1H) 3.53 (m, 2H), 3.39 (m, 2H), 3.29 (m, 3H).

### Step 7: 4-(3-bromo-4-fluorophenyl)-3-(4-((2-methoxyethyl)amino)-1,2,5-oxadiazol-3-yl)-1,2,4-oxadiazol-5(4H)-one 1h

The compound N-(3-bromo-4-fluorophenyl)-N′-hydroxy-4-((2-methoxyethyl)amino)-1,2,5-oxadiazole-3-carboximidamide (3.9 g, 10.4 mmol) was added to ethyl acetate (50 mL). The mixture was heated to 60° C., and 1,1′-carbonyldiimidazole (2.53 g, 15.6 mmol) was added. The mixture was stirred for 30 minutes. The organic phase was washed with 1N HCl and saturated brine, dried over anhydrous sodium sulfate and concentrated in vacuo to obtain the compound 1h (4.0 g, 96%).

### Step 8: 4-(3-bromo-4-fluorophenyl)-3-(4-((2-hydroxyethyl)amino)-1,2,5-oxadiazol-3-yl)-1,2,4-oxadiazol-5(4H)-one 1i

The compound 4-(3-bromo-4-fluorophenyl)-3-(4-((2-methoxyethyl)amino)-1,2,5-oxadiazol-3-yl)-1,2,4-oxadiazol-5(4H)-one 1h (4 g, 10 mmol) was added to dichloromethane (25 mL), and a solution of boron tribromide in dichloromethane (25 mL, 25 mmol) was added dropwise at −78° C. The reaction mixture was stirred at room temperature and monitored by LC-MS. After the raw material was completely converted, the reaction was stopped. Saturated sodium bicarbonate solution was added under an ice bath to adjust the pH to neutral. The organic phase was washed with water and saturated brine, dried over anhydrous sodium sulfate and concentrated in vacuo to obtain the compound 1i (2.0 g, 96%).

MS m/z (ESI): 385.9.

### Step 9: 2-((4-(4-(3-bromo-4-fluorophenyl)-5-oxo-4,5-dihydro-1,2,4-oxadiazol-3-yl)-1,2,5-oxadiazol-3-yl)amino)ethyl methanesulfonate 1j

The compound 4-(3-bromo-4-fluorophenyl)-3-(4-((2-hydroxyethyl)amino)-1,2,5-oxadiazol-3-yl)-1,2,4-oxadiazol-5(4H)-one (2 g, 5.2 mmol) was added to ethyl acetate (15 mL). Methanesulfonyl chloride (593 mg, 5.2 mmol) was added at room temperature, followed by the addition of triethylamine (526 mg, 5.2 mmol). The reaction was monitored by LC-MS. After the raw material was completely converted, the reaction was stopped. The organic phase was washed with water and saturated brine, dried over anhydrous sodium sulfate and concentrated in vacuo to obtain the compound 1j (2.0 g, 82%).

MS m/z (ESI): 463.9.

### Step 10: 3-(4-((2-azidoethyl)amino)-1,2,5-oxadiazol-3-yl)-4-(3-bromo-4-fluorophenyl)-1,2,4-oxadiazol-5(4H)-one 1k

The compound 2-((4-(4-(3-bromo-4-fluorophenyl)-5-oxo-4,5-dihydro-1,2,4-oxadiazol-3-yl)-1,2,5-oxadiazol-3-yl)amino)ethyl methanesulfonate (9.8 g, 21.1 mmol) was added to DMF (45 mL), and sodium azide (1.7 g, 26.4 mmol) was added at room temperature. The mixture was stirred for 4 hours at 50° C. The reaction was monitored by LC-MS. After the raw material was completely converted, the reaction was stopped. Water and ethyl acetate were added. The organic phase was washed with water and saturated brine, dried over anhydrous sodium sulfate and concentrated in vacuo to obtain the compound 1k (9.0 g, 100%).

MS m/z (ESI): 411.0.

### Step 11: 3-(4-((2-aminoethyl)amino)-1,2,5-oxadiazol-3-yl)-4-(3-bromo-4-fluorophenyl)-1,2,4-oxadiazol-5(4H)-one hydroiodide 1l

The compound 3-(4-((2-azidoethyl)amino)-1,2,5-oxadiazol-3-yl)-4-(3-bromo-4-fluorophenyl)-1,2,4-oxadiazole-5(4H)-one (9.0 g, 21.9 mmol) was added to methanol (160 mL). Sodium iodide was added (14.3 g, 131.74 mmol) at room temperature. The mixture was stirred for 5 minutes, and then a solution of trimethylchlorosilane (15.6 mL, 131.7 mmol) in methanol (32 mL) was added dropwise. The reaction mixture was stirred for 4 hours and monitored by LC-MS. After the raw material was completely converted, the reaction was stopped. The reaction solution was poured into an aqueous sodium thiosulfate solution (23 g, 900 mL) in an ice bath. A solid was precipitated, filtered and dried to obtain the compound 11 (10.5 g, 91%).

MS m/z (ESI): 387.0.

### Synthesis of Example Compounds

### Example 1

### (Z)—N¹-(2-((4-(N-(3-bromo-4-fluorophenyl)-N′-hydroxycarbamimidoyl)-1,2,5-oxadiazol-3-yl)amino)ethyl)oxalamide (1)

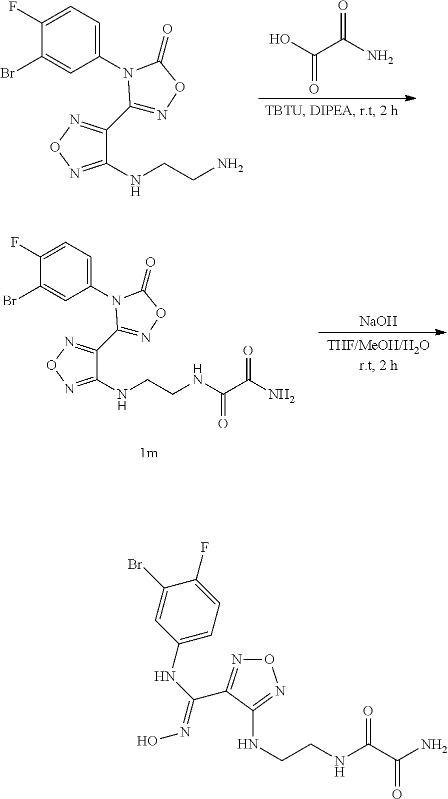 [SMILES: NCCNc1nonc1-c1noc(=O)n1-c1ccc(F)c(Br)c1 ; NC(=O)C(=O)O ; NC(=O)C(=O)NCCNc1nonc1-c1noc(=O)n1-c1ccc(F)c(Br)c1 ; "NaOH" ; NC(=O)C(=O)NCCNc1nonc1/C(=N/O)Nc1ccc(F)c(Br)c1]

### Step 1: N¹-(2-((4-(4-(3-bromo-4-fluorophenyl)-5-oxo-4,5-dihydro-1,2,4-oxadiazol-3-yl)-1,2,5-oxadiazol-3-yl)amino)ethyl)oxalamide 1m

In a 100 mL one-necked flask, 3-(4-((2-aminoethyl)amino)-1,2,5-oxadiazol-3-yl)-4-(3-bromo-4-fluorophenyl)-1,2,4-oxadiazol-5(4H)-one (300 mg, 0.78 mmol) and 2-amino-2-oxoacetic acid (138 mg, 1.56 mmol) were dissolved in N,N-dimethylformamide (8 mL). Then O-Benzotriazole-N,N,N′,N′-tetramethyluronium tetrafluoroborate (375.6 mg, 1.17 mmol) was added, followed by addition of N,N-diisopropylethylamine (0.5 mL, 2.34 mmol). The reaction mixture was stirred at room temperature for 2 hours. Water (50 mL) was added. A solid was precipitated, filtered and dried to obtain N¹-(2-((4-(4-(3-bromo-4-fluorophenyl)-5-carbonyl)-4,5-dihydro-1,2,4-oxadiazol-3-yl)-1,2,5-oxadiazol-3-yl)amino)ethyl)oxalamide 1m (105 mg), yield 32.0%.

MS m/z (ESI): 456.0, 458.0 (M, M+2).

### Step 2: (Z)—N¹-(2-((4-(N-(3-bromo-4-fluorophenyl)-N′-hydroxycarbamimidoyl)-1,2,5-oxadiazol-3-yl)amino)ethyl)oxalamide 1

In a 100 mL one-necked flask, N¹-(2-((4-(4-(3-bromo-4-fluorophenyl)-5-carbonyl-N¹-(2-((4-(4-(3-bromo-4-fluorophenyl)-5-carbonyl-4,5-dihydro-1,2,4-oxadiazol-3-yl)-2,5-oxadiazol-3-yl)amino)ethyl)oxalamide (105 mg, 0.23 mmol) was dissolved in tetrahydrofuran/methanol (5 mL/5 mL), and sodium hydroxide (46 mg 1.15 mmol) dissolved in water (2 mL) was added to the above solution. The reaction mixture was stirred at room temperature for 2 hours and monitored by LC-MS. After the raw material was completely converted, the reaction was stopped. Saturated ammonium chloride solution (30 mL) was added, and the mixture was extracted with ethyl acetate (30 mL×2). The combined organic phases were washed with saturated sodium chloride (30 mL), dried over anhydrous sodium sulfate and filtered. The filtrate was concentrated and purified by preparative silica gel plate (developing solvent:dichloromethane/methanol=10/1; eluent:ethyl acetate/methanol=10/1) to obtain (Z)—(Z)—N¹-(2-((4-(N-(3-bromo-4-fluorophenyl)-N′-hydroxycarbamimidoyl)-1,2,5-3-yl)amino)ethyl)oxalamide 1 (36.6 mg), yield 40.0%.

MS m/z (ESI): 430.0, 432.0 (M, M+2).

¹H NMR (400 MHz, DMSO-d₆, ppm) δ 11.43 (s, 1H), 8.88 (s, 1H), 8.83 (s, 1H), 8.05 (s, 1H), 7.79 (s, 1H), 7.18 (t, J=8.8 Hz, 1H), 7.12 (dd, J₁=6.0 Hz, J₂=2.8 Hz, 1H), 6.75 (m, 1H), 6.30 (t, J=6.0 Hz, 1H), 3.36 (m, 4H).

### Example 2

### (Z)—N¹-(2-((4-(N-(3-bromo-4-fluorophenyl)-N′-hydroxycarbamimidoyl)-1,2,5-oxadiazol-3-yl)amino)ethyl)-N²-methyloxalamide (2)

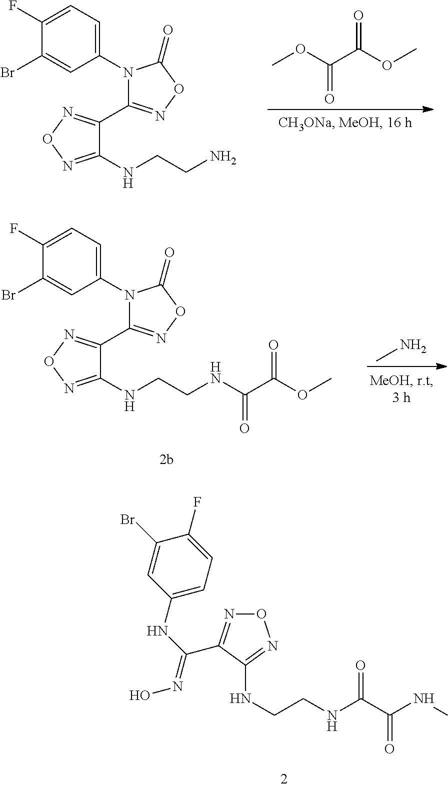

### Step 1: methyl 2-((2-((4-(4-(3-bromo-4-fluorophenyl)-5-oxo-4,5-dihydro-1,2,4-oxadiazol-3-yl)-1,2,5-oxadiazol-3-yl)amino)ethyl)amino)-2-oxoacetate 2b

In a 100 mL one-necked flask, 3-(4-((2-aminoethyl)amino)-1,2,5-oxadiazol-3-yl)-4-(3-bromo-4-fluorophenyl)-1,2,4-oxadiazol-5(4H)-one (385 mg, 1.0 mmol) and dimethyl oxalate (141.6 mg, 1.2 mmol) were dissolved in methanol (15 mL), and then sodium methoxide (130 mg, 2.4 mmol) was added. The reaction mixture was stirred overnight at room temperature and monitored by LC-MS. After the raw material was completely converted, the reaction was stopped. Saturated ammonium chloride solution (30 mL) was added, and the mixture was extracted with ethyl acetate (50 mL×2). The combined organic phases were washed with saturated sodium chloride (50 mL), dried over anhydrous sodium sulfate and filtered. The filtrate was concentrated to obtain methyl 2-((2-((4-(4-(3-bromo-4-fluorophenyl)-5-oxo-4,5-dihydro-1,2,4-oxadiazol-3-yl)-1,2,5-oxadiazol-3-yl)amino)ethyl)amino)-2-oxoacetate 2b (200 mg), yield 50.0%.

MS m/z (ESI): 471.0, 473.0 (M, M+2).

### Step 2: (Z)—N¹-(2-((4-(N-(3-bromo-4-fluorophenyl)-N′-hydroxycarbamimidoyl)-1,2,5-oxadiazol-3-yl)amino)ethyl)-N²-methyloxalamide 2

In a 100 mL one-necked flask, methyl 2-((2-((4-(4-(3-bromo-4-fluorophenyl)-5-oxo-4,5-dihydro-1,2,4-oxadiazol-3-yl)-1,2,5-oxadiazol-3-yl)amino)ethyl)amino)-2-oxoacetate (200 mg, 0.42 mmol) was dissolved in methanol (5 mL), and then 40% methylamine solution (2 mL) was added to the above solution. The reaction mixture was stirred at room temperature for 3 hours, and monitored by LC-MS. After the raw material was completely converted, the reaction was stopped. Water (30 mL) was added, and the mixture was extracted with ethyl acetate (30 mL×2). The combined organic phases were washed with saturated sodium chloride (30 mL), dried over anhydrous sodium sulfate and filtered. The filtrate was concentrated and purified by preparative silica gel plate (developing solvent: dichloromethane/methanol=10/1; eluent: ethyl acetate/methanol=10/1) to obtain (Z)—N¹-(2-((4-(N-(3-bromo-4-fluorophenyl)-N′-hydroxycarbamidoyl)-1,2,5-oxadiazol-3-yl)amino)ethyl)-N²-methyloxalamide 2 (57.5 mg), yield: 29.6%.

MS m/z (ESI): 444.0, 446.0 (M, M+2).

¹H NMR (400 MHz, DMSO-d₆, ppm) δ 11.42 (s, 1H), 8.88 (s, 1H), 8.86 (m, 1H), 8.68 (m, 1H), 7.18 (t, J=8.8 Hz, 1H), 7.10 (dd, J₁=6.0 Hz, J₂=2.8 Hz, 1H), 6.74 (m, 1H), 6.30 (t, J=6.0 Hz, 1H), 3.38 (m, 4H), 2.66 (d, J=4.0 Hz, 3H).

### Example 3

### (Z)—N¹-(2-((4-(N-(3-bromo-4-fluorophenyl)-N′-hydroxycarbamimidoyl)-1,2,5-oxadiazol-3-yl)amino)ethyl)-N²-ethyloxalamide (3)

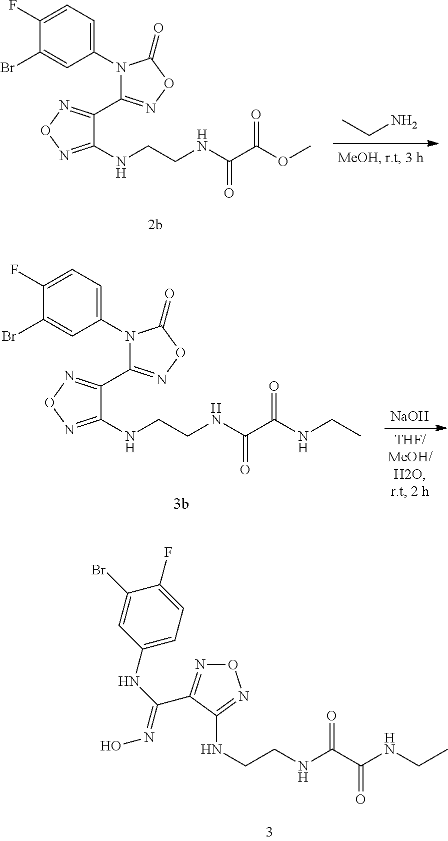

### Step 1: N¹-(2-((4-(4-(3-bromo-4-fluorophenyl)-5-oxo-4,5-dihydro-1,2,4-oxadiazol-3-yl)-1,2,5-oxadiazol-3-yl)amino)ethyl)-N²-ethyloxalamide 3b

In a 100 mL one-necked flask, methyl 2-((2-((4-(4-(3-bromo-4-fluorophenyl)-5-2-((2-((4-(4-(3-bromo-4-fluorophenyl)-5-oxo-4,5-dihydro-1,2,4-oxadiazol-3-yl)-1,2,5-xadiazol-3-yl)amino)ethyl)amino)-2-oxoacetate (240 mg, 0.51 mmol) was dissolved in methanol (15 mL), and then 1M ethylamine solution (2 mL) was added to the above solution. The reaction mixture was stirred at room temperature for 3 hours and monitored by LC-MS. After the raw material was completely converted, the reaction was stopped. Water (30 mL) was added, and the mixture was extracted with ethyl acetate (30 mL×2). The combined organic phases were washed with saturated sodium chloride (30 mL), dried over anhydrous sodium sulfate and filtrated. The filtrate was concentrated to obtain N¹-(2-((4-(4-(3-bromo-4-fluorophenyl)-5-oxo-4,5-dihydro-1,2,4-N¹-(2-((4-(4-(3-bromo-4-fluorophenyl)-5-oxo-4,5-dihydro-1,2,4-oxadiazol-3-yl)-1,2,5-xadiazol-3-yl)amino)ethyl)-N²-ethyloxalamide 3b (190 mg), yield 78.5%.

MS m/z (ESI): 471.0, 473.0 (M, M+2).

### Step 2: (Z)—N¹-(2-((4-(N-(3-bromo-4-fluorophenyl)-N′-hydroxycarbamimidoyl)-1,2,5-oxadiazol-3-yl)amino)ethyl)-N²-ethyloxalamide 3

In a 100 mL one-necked flask, N¹-(2-((4-(4-(3-bromo-4-fluorophenyl)-5-oxo-4,5-dihydro-1,2,4-oxadiazol-3-yl)-1,2,5-oxadiazol-3-yl)amino)ethyl)-N²-ethyloxalamide (190 mg, 0.39 mmol) was dissolved in tetrahydrofuran/methanol (8 mL/8 mL), and then sodium hydroxide (62.7 mg, 1.57 mmol) dissolved in water (4 mL) was added to the above solution. The reaction mixture was stirred at room temperature for 2 hours and monitored by LC-MS. After the raw material was completely converted, the reaction was stopped. Saturated ammonium chloride solution (30 mL) was added, and the mixture was extracted with ethyl acetate (30 mL×2). The combined organic phases were washed with saturated sodium chloride (30 mL), dried over anhydrous sodium sulfate and filtered. The filtrate was concentrated and purified by preparative silica gel plate (developing solvent: dichloromethane/methanol=10/1; eluent: ethyl acetate/methanol=10/1) to obtain (Z)—N¹-(2-((4-(N-(3-bromo-4-fluorophenyl)-N′-hydroxycarbamidoyl)-1,2,5-oxadiazol-3-yl)amino)ethyl)-N²-ethyloxalamide 3 (80.0 mg), yield 43.1%.

MS m/z (ESI): 458.0, 460.0 (M, M+2).

¹H NMR (400 MHz, DMSO-d₆, ppm) δ 11.42 (s, 1H), 8.88 (s, 1H), 8.86 (m, 1H), 8.75 (t, J=6.0 Hz, 1H), 7.18 (t, J=8.8 Hz, 1H), 7.10 (dd, J₁=6.0 Hz, J₂=2.4 Hz, 1H), 6.74 (m, 1H), 6.31 (t, J=6.0 Hz, 1H), 3.38 (m, 4H), 3.15 (m, 2H), 1.04 (m, 3H).

### Example 4

### (Z)—N¹-benzyl-N²-(2-((4-(N-(3-bromo-4-fluorophenyl)-N′-hydroxycarbamimidoyl)-1,2,5-oxadiazol-3-yl)amino)ethyl)oxalamide (4)

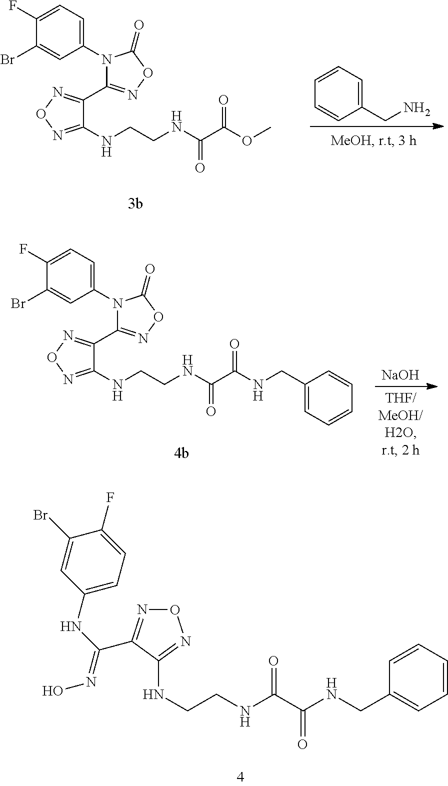

### Step 1: N¹-benzyl-N²-(2-((4-(4-(3-bromo-4-fluorophenyl)-5-oxo-4,5-dihydro-1,2,4-oxadiazol-3-yl)-1,2,5-oxadiazol-3-yl)amino)ethyl)oxalamide 4b

In a 100 mL one-necked flask, methyl 2-((2-((4-(4-(3-bromo-4-fluorophenyl)-5-2-((2-((4-(4-(3-bromo-4-fluorophenyl)-5-oxo-4,5-dihydro-1,2,4-oxadiazol-3-yl-1,2,5-adiazol-3-yl)amino)ethyl)amino)-2-oxoacetate (200 mg, 0.42 mmol) was dissolved in methanol (15 mL), and then benzylamine (1 mL) was added to the above solution. The reaction mixture was stirred at room temperature for 2 hours and monitored by LC-MS. After the raw material was completely converted, the reaction was stopped. Ethyl acetate (50 mL) was added, and the mixture was washed with 1N hydrochloric acid (30 mL×2) and saturated sodium chloride (30 mL). The organic phase was dried over anhydrous sodium sulfate and filtrated. The filtrate is concentrated to obtain N¹-benzyl-N¹-benzyl-N²-(2-((4-(4-(3-bromo-4-fluorophenyl)-5-oxo-4,5-dihydro-1,2,4-oxadiazol-1-yl)-1,2,5-oxadiazol-3-yl)amino)ethyl)oxalamide 4b (190 mg), yield 82.0%.

MS m/z (ESI): 546.0, 548.0 (M, M+2).

### Step 2: (Z)—N¹-benzyl-N²-(2-((4-(N-(3-bromo-4-fluorophenyl)-N′-hydroxycarbamimidoyl)-1,2,5-oxadiazol-3-yl)amino)ethyl)oxalamide 4

In a 100 mL one-necked flask, N¹-benzyl-N²-(2-((4-(4-(3-bromo-4-fluorophenyl)-5-oxo-4,5-dihydro-1,2,4-oxadiazol-3-yl)-1,2,5-oxadiazol-3-yl)amino)ethyl)oxalamide (190 mg, 0.35 mmol) was dissolved in tetrahydrofuran/methanol (8 mL/8 mL), and then sodium hydroxide (100 mg, 2.5 mmol) dissolved in water (4 mL) was added to the above solution. The reaction mixture was stirred at room temperature for 2 hours and monitored by LC-MS. After the raw material was completely converted, the reaction was stopped. Saturated ammonium chloride solution (30 mL) was added, and the mixture was extracted with ethyl acetate (30 mL×2). The combined organic phases were washed with saturated sodium chloride (30 mL), dried over anhydrous sodium sulfate, and filtered. The filtrate was concentrated and purified by preparative silica gel plate (developing solvent: dichloromethane/methanol=10/1; eluent: ethyl acetate/methanol=10/1) to obtain (Z)—N¹-benzyl-N²-(2-((4-(N-(3-bromo-4-fluorophenyl)-N′-hydroxycarbamimidoyl)-1,2,5-oxadiazol-3-yl)amino)ethyl)oxalamide 4 (60.9 mg), yield 39.0%.

MS m/z (ESI): 520.0, 522.0 (M, M+2).

¹H NMR (400 MHz, DMSO-d₆, ppm) δ 11.42 (s, 1H), 9.35 (t, J=6.0 Hz, 1H), 8.87 (m, 2H), 7.30 (m, 2H), 7.24 (m, 2H), 6.74 (m, 1H), 6.33 (t, J=6.0 Hz, 1H), 4.33 (d, J=6.4 Hz, 2H), 3.38 (m, 4H).

### Example 5

### (Z)—N-(2-((4-(N-(3-bromo-4-fluorophenyl)-N′-hydroxycarbamimidoyl)-1,2,5-oxadiazol-3-yl)amino)ethyl)-2-morpholino-2-oxoacetamide (5)

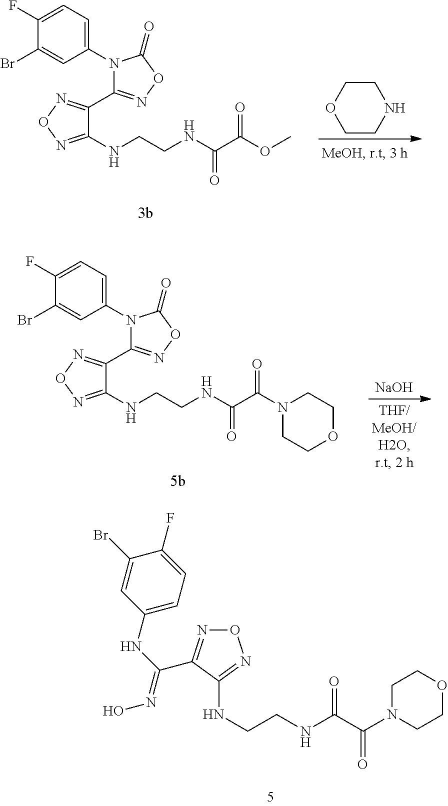

### Step 1: N-(2-((4-(4-(3-bromo-4-fluorophenyl)-5-oxo-4,5-dihydro-1,2,4-oxadiazol-3-yl)-1,2,5-oxadiazol-3-yl)amino)ethyl)-2-morpholino-2-oxoacetamide 5b

In a 100 mL one-necked flask, methyl 2-((2-((4-(4-(3-bromo-4-fluorophenyl)-5-oxo-4,5-dihydro-1,2,4-oxadiazol-3-yl)-1,2,5-oxadiazol-3-yl)amino)ethyl)amino)-2-oxoacetate (170 mg, 0.36 mmol) was dissolved in methanol (15 mL), and then morpholine (1 mL) was added to the above solution. The reaction mixture was stirred at room temperature for 3 hours and monitored by LC-MS. After the raw material was completely converted, the reaction was stopped. Ethyl acetate (50 mL) was added, and the mixture was washed with 1N hydrochloric acid (30 mL×2) and saturated sodium chloride (30 mL). The organic phase was dried over anhydrous sodium sulfate and filtered. The filtrate was concentrated to obtain N-(2-((4-(4-(3-bromo-4-fluorophenyl)-5-oxo-4,5-dihydro-1,2,4-oxadiazol-3-yl)-1,2,5-oxadiazol-3-yl)amino)ethyl)-2-morpholino-2-oxoacetamide 5b (120 mg), yield 63.4%.

MS m/z (ESI): 526.0, 528.0 (M, M+2).

### Step 2: (Z)—N-(2-((4-(N-(3-bromo-4-fluorophenyl)-N′-hydroxycarbamimidoyl)-1,2,5-oxadiazol-3-yl)amino)ethyl)-2-morpholino-2-oxoacetamide 5

In a 100 mL one-necked flask, N-(2-((4-(4-(3-bromo-4-fluorophenyl)-5-oxo-4,5-dihydro-1,2,4-oxadiazol-3-yl)-1,2,5-oxadiazol-3-yl)amino)ethyl)-2-morpholino-2-oxoacetamide (120 mg, 0.23 mmol) was dissolved in tetrahydrofuran/methanol (8 mL/8 mL), and then sodium hydroxide (50 mg, 1.25 mmol) dissolved in water (4 mL) was added to the above solution. The reaction mixture was stirred at room temperature for 2 hours and monitored by LC-MS. After the raw material was completely converted, the reaction was stopped. Saturated ammonium chloride solution (30 mL) was added, and the mixture was extracted with ethyl acetate (30 mL×2). The combined organic phases were washed with saturated sodium chloride (30 mL), dried over sodium sulfate and filtered. The filtrate was concentrated and purified by preparative silica gel plate (developing solvent: dichloromethane/methanol=10/1; eluent: ethyl acetate/methanol=10/1) to obtain (Z)—N-(2-((4-(N-(3-bromo-4-fluorophenyl)-N′-hydroxycarbamimidoyl)-1,2,5-oxadiazol-3-yl)amino)ethyl)-2-morpholino-2-oxoacetamide (30.7 mg), yield 30.1%.

MS m/z (ESI): 500.0, 502.0 (M, M+2).

¹H NMR (400 MHz, DMSO-d₆, ppm) δ 11.45 (s, 1H), 8.89 (s, 1H), 8.83 (m, 1H), 7.20 (t, J=8.8 Hz, 1H), 7.11 (dd, J₁=6.0 Hz, J₂=2.8 Hz, 1H), 6.77 (m, 1H), 6.24(t, J=6.0 Hz, 1H), 3.58 (m, 4H), 3.48 (m, 4H), 3.36 (m, 4H).

### Example 6

### (Z)—N¹-(2-((4-(N-(3-bromo-4-fluorophenyl)-N′-hydroxycarbamimidoyl)-1,2,5-oxadiazol-3-yl)amino)ethyl)-N²-methoxyoxalamide (6)

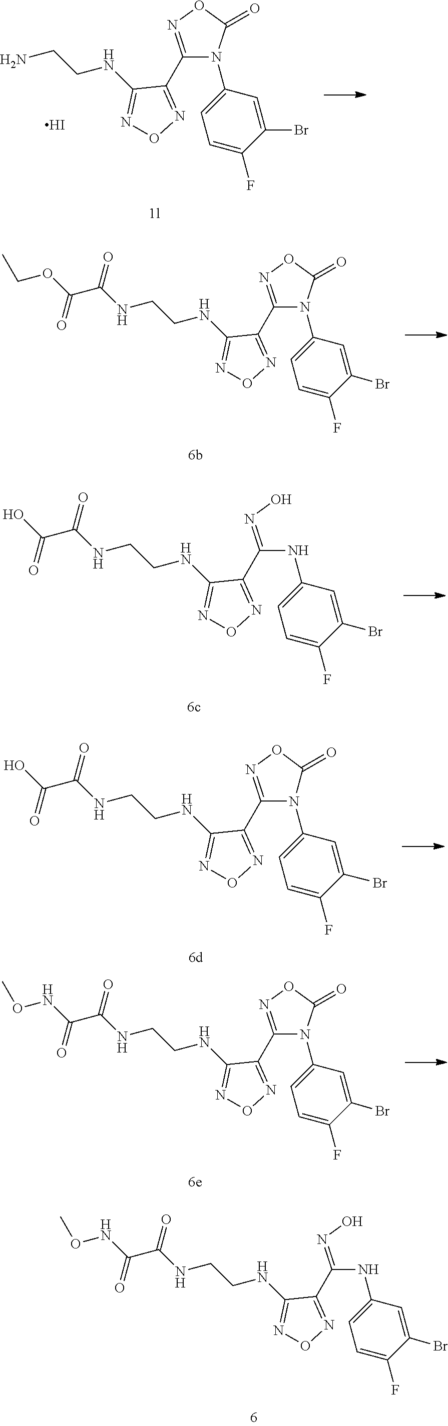

### Step 1: ethyl 2-((2-((4-(4-(3-bromo-4-fluorophenyl)-5-oxo-4,5-dihydro-1,2,4-2-((2-((4-(4-(3-bromo-4-fluorophenyl)-5-oxo-4,5-dihydro-1,2,4-oxadiazol-3-yl)-oxadiazol-3-yl)amino)ethyl)amino)-2-oxoacetate 6b

In a 100 mL one-necked flask, 3-(4-((2-aminoethyl)amino)-1,2,5-oxadiazol-3-yl)-4-(3-bromo-4-fluorophenyl)-1,2,4-oxadiazol-5 (4H)-one hydroiodide 1l (2.5 g, 4.88 mmol) was dissolved in tetrahydrofuran (30 mL). Ethyl 2-chloro-2-oxoacetate (730 mg, 5.37 mmol) was added under an ice bath, followed by the addition of triethylamine (1.23 g, 12.2 mmol). The mixture was stirred for 2 hours. Water (50 mL) was added, and then a solid was precipitated. The mixture was extracted with ethyl acetate (50 mL×2). The combined organic phases were washed with saturated brine, dried over anhydrous sodium sulfate, filtered and subjected to flash column chromatography to obtain ethyl 2-((2-((4-(4-(3-bromo-4-fluorophenyl)-5-oxo-4,5-dihydro-1,2,4-oxadiazol-3-yl)-1,2,5-oxadiazol-3-yl)amino)ethyl)amino)-2-oxoacetate 6b (1.1 g), yield 46.5%.

MS m/z (ESI): 484.9 (M, M+H)⁺.

### Step 2: (Z)-2-((2-((4-(N-(3-bromo-4-fluorophenyl)-N′-hydroxycarbamimidoyl)-1,2,5-oxadiazol-3-yl)amino)ethyl)amino)-2-oxoacetic acid 6c

In a 100 mL one-necked flask, ethyl 2-((2-((4-(4-(3-bromo-4-fluorophenyl)-5-oxo-4,5-dihydro-1,2,4-oxadiazol-3-yl)-1,2,5-oxadiazol-3-yl)amino)ethyl)amino)-2-oxoacetate (700 mg, 1.44 mmol) was dissolved in ethanol (10 mL), and then 2N sodium hydroxide (1 mL, 2.0 mmol) was added. The mixture was stirred at 90° C. for 3 hours. The reaction was monitored by LC-MS until the raw material was completely converted. The reaction solution was concentrated, extracted with ethyl acetate, washed with water and saturated brine, dried over anhydrous sodium sulfate and filtered. The filtrate was concentrated to obtain (Z)-2-((2-((4-(N-(3-bromo-4-fluorophenyl)-N′-hydroxycarbamimidoyl)-1,2,5-oxadiazol-3-yl)amino)ethyl)amino)-2-oxoacetic acid 6c (600 mg), yield: 97.0%.

MS m/z (ESI): 429.0 (M−H)⁻.

### Step 3: 2-((2-((4-(4-(3-bromo-4-fluorophenyl)-5-oxo-4,5-dihydro-1,2,4-oxadiazol-3-yl)-1,2,5-oxadiazol-3-yl)amino)ethyl)amino)-2-oxoacetic acid 6d

In a 50 mL one-necked flask, (Z)-2-((2-((4-(N-(3-bromo-4-fluorophenyl)-N′-hydroxycarbamimidoyl)-1,2,5-oxadiazol-3-yl)amino)ethyl)amino)-2-oxoacetic acid (600 mg, 1.39 mmol) was dissolved in ethyl acetate (20 mL), and then CDI (271 mg, 1.67 mmol) was added. The mixture was stirred at 60° C. for 1 hour. The reaction was monitored by LC-MS until the raw material was completely converted. The mixture was washed with 1N hydrochloric acid, water and saturated brine, dried over anhydrous sodium sulfate and filtered. The filtrate was concentrated to obtain 2-((2-((4-(4-(3-bromo-4-fluorophenyl)-5-oxo-4,5-dihydro-1,2,4-oxadiazol-3-yl)-1,2,5-oxadiazol-3-yl)amino)ethyl)amino)-2-oxoacetic acid 6d (370 mg), yield 58.3%.

MS m/z (ESI): 454.9 (M−H)⁻.

### Step 4: N¹-(2-((4-(4-(3-bromo-4-fluorophenyl)-5-oxo-4,5-dihydro-1,2,4-oxadiazol-3-yl)-1,2,5-oxadiazol-3-yl)amino)ethyl)-N²-methoxyoxalamide 6e

In a 50 mL one-necked flask, 2-((2-((4-(4-(3-bromo-4-fluorophenyl)-5-oxo-4,5-dihydro-1,2,4-oxadiazol-3-yl)-1,2,5-oxadiazol-3-yl)amino)ethyl)amino)-2-oxoacetic acid (100 mg, 0.22 mmol) was dissolved in DMF (5 mL), and then O-methylhydroxylamine hydrochloride (20 mg, 0.22 mmol), HATU (130 mg, 0.33 mmol) and DIPEA (70 mg, 0.55 mmol) were added. The mixture was stirred at room temperature overnight. The reaction was monitored by LC-MS until the raw material was completely converted. The mixture was extracted with ethyl acetate, washed with water and saturated brine, dried over anhydrous sodium sulfate and filtered. The filtrate was concentrated and subjected to flash column chromatography to obtain N¹-(2-((4-(4-(3-bromo-4-fluorophenyl)-5-oxo-4,5-dihydro-1,2,4-oxadiazol-3-yl)-1,2,5-oxadiazol-3-yl)amino)ethyl)-N²-methoxyoxalamide 6e (30 mg), yield 28.1%.

MS m/z (ESI): 484.0 (M−H)⁻.

### Step 5: (Z)—N¹-(2-((4-(N-(3-bromo-4-fluorophenyl)-N′-hydroxycarbamimidoyl)-1,2,5-oxadiazol-3-yl)amino)ethyl)-N²-methoxyoxalamide 6

In a 50 mL one-necked flask, N¹-(2-((4-(4-(3-bromo-4-fluorophenyl)-5-oxo-4,5-N¹-(2-((4-(4-(3-bromo-4-fluorophenyl)-5-oxo-4,5-dihydro-1,2,4-oxadiazol-3-yl)-1,2,5-xadiazol-3-yl)amino)ethyl)-N²-methoxyoxylalamide (30 mg, 0.06 mmol) was dissolved in ethanol (5 mL), and then 2N sodium hydroxide (0.3 mL, 0.6 mmol) was added. The reaction mixture was stirred overnight at room temperature. The reaction was monitored by LC-MS until the raw material was completely converted. The mixture was extracted with ethyl acetate, washed with water and saturated brine, dried over anhydrous sodium sulfate and filtered. The filtrate was concentrated and subjected to flash thin-layer chromatography to obtain (Z)—N¹-(2-((4-(N-(3-bromo-4-fluorophenyl)-N′—(Z)—N¹-(2-((4-(N-(3-bromo-4-fluorophenyl)-N′-hydroxycarbamimidoyl)-1,2,5-3-yl)amino)ethyl)-N²-methoxyoxalamide 6 (6.0 mg), yield 21.7%.

MS m/z (ESI): 457.9.0 (M−H)⁻.

¹H NMR (400 MHz, CD₃OD, ppm) δ7.0 (m, 1H), 6.94 (m, 1H), 6.74 (m, 1H), 3.63 (s, 3H), 3.42 (4, 2H), 3.33 (m, 2H).

### Example 7

### (Z)—N¹-(2-((4-(N-(3-bromo-4-fluorophenyl)-N′-hydroxycarbamimidoyl)-1,2,5-oxadiazol-3-yl)amino)ethyl)-N²-cyclopropyloxalamide (7)

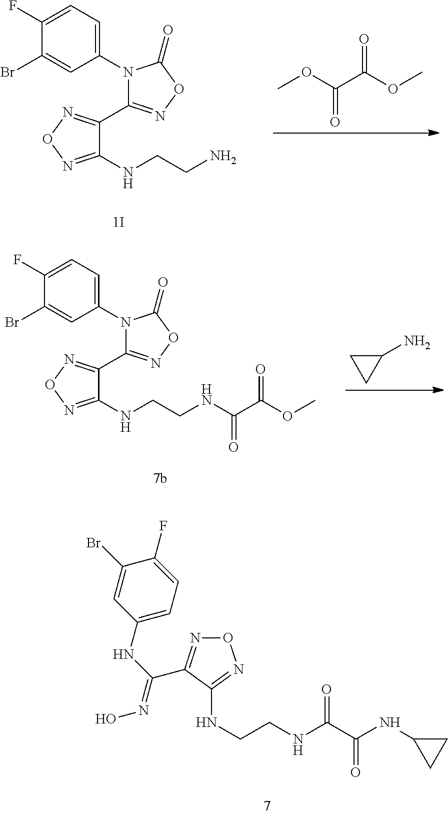

### Step 1: methyl 2-((2-((4-(4-(3-bromo-4-fluorophenyl)-5-oxo-4,5-dihydro-1,2,4-oxadiazol-3-yl)-1,2,5-oxadiazol-3-yl)amino)ethyl)amino)-2-oxoacetate 7b

In a 100 mL one-necked flask, 3-(4-((2-aminoethyl)amino)-1,2,5-oxadiazol-3-yl)-4-(3-bromo-4-fluorophenyl)-1,2, 4-oxadiazol-5(4H)-one (385 mg, 1.0 mmol) and dimethyl oxalate (141.6 mg, 1.2 mmol) were dissolved in methanol (15 mL), and then sodium methoxide (130 mg, 2.4 mmol) was added. The reaction mixture was stirred at room temperature overnight and monitored by LC-MS. After the raw material was completely converted, the reaction was stopped. Saturated ammonium chloride solution (30 mL) was added, and the mixture was extracted with ethyl acetate (50 mL×2). The combined organic phases were washed with saturated sodium chloride (50 mL), dried over anhydrous sodium sulfate and filtered. The filtrate was concentrated to obtain methyl 2-((2-((4-(4-(3-bromo-4-fluorophenyl)-5-oxo-4,5-dihydro-1,2,4-oxadiazol-3-yl))-1,2,5-oxadiazol-3-yl)amino)ethyl)amino)-2-oxoacetate 7b (200 mg), yield 50.0%.

MS m/z (ESI): 471.0, 473.0 (M, M+2).

### Step 2: (Z)—N¹-(2-((4-(N-(3-bromo-4-fluorophenyl)-N′-hydroxycarbamimidoyl)-1,2,5-oxadiazol-3-yl)amino)ethyl)-N²-cyclopropyloxalamide 7

In a sealed tube, methyl 2-((2-((4-(4-(3-bromo-4-fluorophenyl)-5-oxo-4,5-dihydro-2-((2-((4-(4-(3-bromo-4-fluorophenyl)-5-oxo-4,5-dihydro-1,2,4-oxadiazol-3-yl)-1,2,5-xadiazol-3-yl)amino)ethyl)amino)-2-oxoacetate (100 mg, 0.21 mmol) was dissolved in ethanol (5 mL), and then cyclopropylamine (0.5 mL) was added to the above solution. The reaction mixture was stirred overnight at 90° C. and monitored by LC-MS. After the raw material was completely converted, the reaction was stopped. Water (30 mL) was added, and the mixture was extracted with ethyl acetate (30 mL×2). The combined organic phases were washed with saturated sodium chloride (30 mL), dried over anhydrous sodium sulfate and filtered. The filtrate was concentrated and purified by preparative silica gel plate to obtain (Z)—N¹-(2-((4-(N-(3-bromo-4-fluorophenyl)-N′—(Z)—N¹-(2-((4-(N-(3-bromo-4-fluorophenyl)-N′-hydroxycarbamimidoyl)-1,2,5-3-yl)amino)ethyl)-N²-cyclopropyloxalamide 7 (30 mg), yield 30.3%.

MS m/z (ESI): 470.0 (M+H)⁺.

¹H NMR (400 MHz, CD₃OD, ppm) δ 8.87 (s, 1H), 8.77 (s, 1H), 7.18 (m, 1H), 7.15 (m, 1H), 6.72 (m, 1H), 6.34 (m, 1H), 2.75 (m, 1H), 0.62 (m, 4H).

### Example 8

### (Z)—N′-(2-((4-(N-(3-bromo-4-fluorophenyl)-N′-hydroxycarbamimidoyl)-1,2,5-oxadiazol-3-yl)amino)ethyl)-N²-hydroxyoxalamide (8)

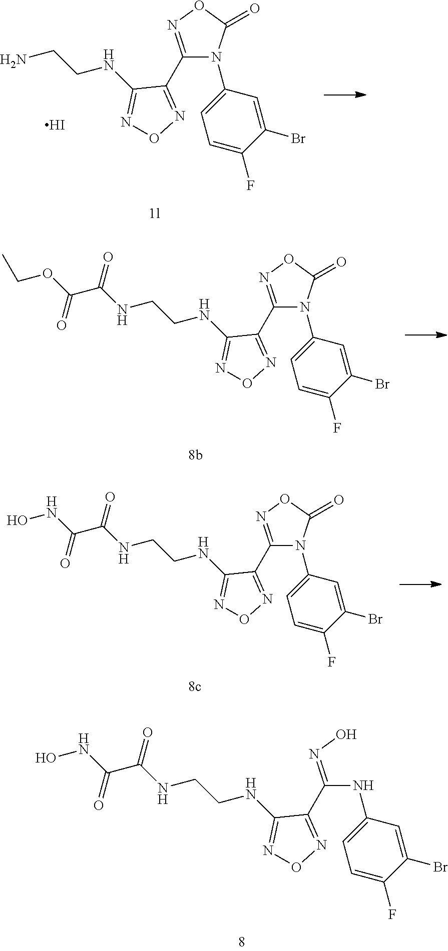

### Step 1: ethyl 2-((2-((4-(4-(3-bromo-4-fluorophenyl)-5-oxo-4,5-dihydro-1,2,4-oxadiazol-3-yl)-1,2,5-oxadiazol-3-yl)amino)ethyl)amino)-2-oxoacetate 8b

In a 100 mL one-necked flask, 3-(4-((2-aminoethyl)amino)-1,2,5-oxadiazol-3-yl)-4-(3-bromo-4-fluorophenyl)-1,2,4-oxadiazol-5(4H)-one hydroiodide 1l (2.5 g, 4.88 mmol) was dissolved in tetrahydrofuran (30 mL), and then ethyl 2-chloro-2-carbonylacetate (730 mg, 5.37 mmol) was added under an ice bath, followed by addition of triethylamine (1.23 g, 12.2 mmol). The reaction mixture was stirred for 2 hours. Water (50 mL) was added, and a solid was precipitated. The mixture was extracted with ethyl acetate (50 mL×2). The combined organic phases were washed with saturated brine, dried over anhydrous sodium sulfate, filtered and subjected to flash column chromatography to obtain the compound ethyl 2-((2-((4-(4-(3-bromo-4-fluorophenyl)-5-oxo-4,5-dihydro-1,2,4-oxadiazol-3-yl)-1,2,5-oxadiazol-3-yl)amino)ethyl)amino)-2-oxoacetate 8b (1.1 g), yield 46.5%.

MS m/z (ESI): 484.9 (M, M+H)⁺.

### Step 2: N¹-(2-((4-(4-(3-bromo-4-fluorophenyl)-5-oxo-4,5-dihydro-1,2,4-oxadiazol-3-yl)-1,2,5-oxadiazol-3-yl)amino)ethyl)-N²-hydroxyoxalamide 8c

In a one-necked flask, ethyl 2-((2-((4-(4-(3-bromo-4-fluorophenyl)-5-oxo-4,5-dihydro-1,2,4-oxadiazol-3-yl)-1,2,5-oxadiazol-3-yl)amino)ethyl)amino)-2-oxoacetate (70 mg, 0.14 mmol) was dissolved in methanol (5 mL), and then 50% aqueous hydroxylamine solution (0.1 mL) was added to the above solution under an ice bath, followed by the dropwise addition of a saturated solution of sodium hydroxide in methanol (0.2 mL). The reaction mixture was stirred at 0° C. for 30 minutes and monitored by LC-MS. After the raw material was completely converted, the reaction was stopped. The mixture was concentrated, and then 2N hydrochloric acid was added to adjust the pH to neutral. Water was added, and the mixture was extracted with ethyl acetate (30 mL×2). The combined organic phases were washed with saturated sodium chloride (30 mL), dried over anhydrous sodium sulfate and filtered. The filtrate was concentrated to obtain N¹-(2-((4-(4-(3-bromo-4-fluorophenyl)-5-oxo-4,5-dihydro-1,24-oxadiazol-3-yl)-1,2,5-oxadiazol-3-yl)amino)ethyl)-N²-hydroxyoxalamide 8c (60 mg).

MS m/z (ESI): 472.0 (M+H)⁺.

### Step 3: (Z)—N¹-(2-((4-(N-(3-bromo-4-fluorophenyl)-N′-hydroxycarbamimidoyl)-1,2,5-oxadiazol-3-yl)amino)ethyl)-N²-hydroxyoxalamide 8

In a 50 mL one-necked flask, N¹-(2-((4-(4-(3-bromo-4-fluorophenyl)-5-oxo-4,5-dihydro-1,2,4-oxadiazol-3-yl)-1,2,5-oxadiazol-3-yl)amino)ethyl)-N²-hydroxyoxalamide (60 mg, 0.13 mmol) was dissolved in ethanol (5 mL), and then 2N sodium hydroxide (0.2 mL, 0.4 mmol) was added. The mixture was stirred overnight at room temperature. The reaction was monitored by LC-MS until the raw material was completely converted. The mixture was extracted with ethyl acetate, washed with water and saturated brine, dried over anhydrous sodium sulfate and filtered. The filtrate was concentrated and subjected to flash thin-layer chromatography to obtain (Z)—N¹-(2-((4-(N-(3-bromo-4-fluorophenyl)-N′-hydroxycarbamidoyl)-1,2,5-oxadiazol-3-yl)amino)ethyl)-N²-hydroxyoxalamide 8 (24.0 mg), yield 41.5%.

MS m/z (ESI): 444.0 (M−H)⁻.

¹H NMR (400 MHz, MeCD₃OD, ppm) δ 7.04 (m, 1H), 6.94 (m, 1H), 6.71 (m, 1H), 3.43 (m, 2H), 3.36 (m, 2H).

### Example 9

### (Z)—N′-(2-((4-(N-(3-bromo-4-fluorophenyl)-N′-hydroxycarbamimidoyl)-1,2,5-oxadiazol-3-yl)amino)propyl)oxalamide (9)

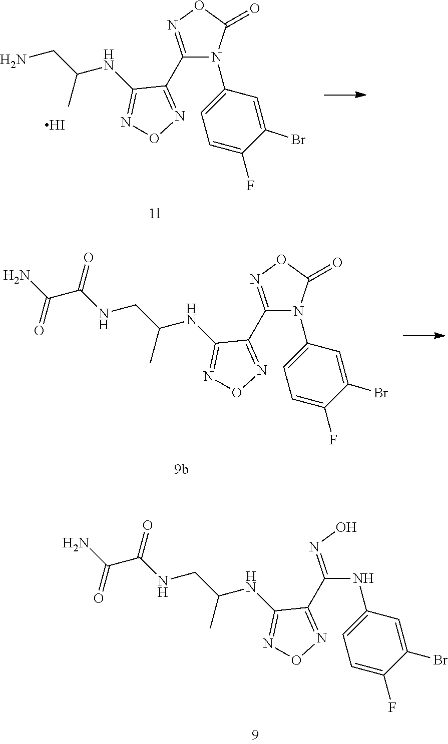

### Step 1: N¹-(2-((4-(4-(3-bromo-4-fluorophenyl)-5-oxo-4,5-dihydro-1,2,4-oxadiazol-3-yl)-1,2,5-oxadiazol-3-yl)amino)propyl)oxalamide 9b

In a 25 mL one-necked flask, 3-(4-((1-aminopropan-2-yl)amino)-1,2,5-oxadiazol-3-yl)-4-(3-bromo-4-fluorophenyl)-1,2,4-oxadiazol-5(4H)-one hydroiodination (2.5 g, 4.88 mmol) was dissolved in DMF (3 mL), and then 2-amino-2-carbonylacetic acid (18.6 mg, 0.21 mmol) was added, followed by addition of HATU (108 mg, 0.29 mmol) and DIPEA (49 mg, 0.38 mmol). The reaction mixture was stirred overnight at room temperature. Water (50 mL) was added, and a solid was precipitated. The mixture was extracted with ethyl acetate (15 mL×2). The combined organic phases were washed with saturated brine, dried over anhydrous sodium sulfate, filtered, and concentrated to obtain N¹-(2-((4-(4-(3-bromo-4-fluorophenyl)-5-oxo-4,5-dihydro-1,2,4-oxadiazol-3-yl)-1,2,5-oxadiazol-3-yl)amino)propyl)oxalamide 9b (36 mg), yield 40.0%.

MS m/z (ESI): 470.0 (M+H)⁺.

### Step 2: (Z)—N¹-(2-((4-(N-(3-bromo-4-fluorophenyl)-N′-hydroxycarbamimidoyl)-1,2,5-oxadiazol-3-yl)amino)propyl)oxalamide 9

In a 50 mL one-necked flask, N¹-(2-((4-(4-(3-bromo-4-fluorophenyl)-5-oxo-4,5-dihydro-1,2,4-oxadiazol-3-yl)-1,2,5-oxadiazol-3-yl)amino)propyl)oxalamide (36 mg, 0.07 mmol) was dissolved in ethanol (5 mL), and then 2N sodium hydroxide (0.2 mL, 0.4 mmol) was added. The reaction mixture was stirred overnight at room temperature. The reaction was monitored by LC-MS until the raw material was completely converted. The mixture was extracted with ethyl acetate, washed with water and saturated brine, dried over anhydrous sodium sulfate and filtered. The filtrate was concentrated and subjected to flash thin-layer chromatography to obtain (Z)—N¹-(2-((4-(N-(3-bromo-4-fluorophenyl)-N′-hydroxycarbamidoimidyl)-1,2,5-oxadiazol-3-yl)amino)propyl)oxalamide 9 (13.0 mg), yield 41.8%.

MS m/z (ESI): 444.0 (M+H)⁺.

¹H NMR (400 MHz, CD₃OD, ppm) δ7.02 (m, 1H), 6.94 (m, 1H), 6.71 (m, 1H), 3.70 (m, 1H), 3.36 (m, 2H), 1.15 (m, 3H).

### Example 10

### (Z)—N-(3-bromo-4-fluorophenyl)-N′-hydroxy-4-((4-(sulfamoylamino)cyclohexyl)amino)-1,2,5-oxadiazole-3-carboximidamide (10)

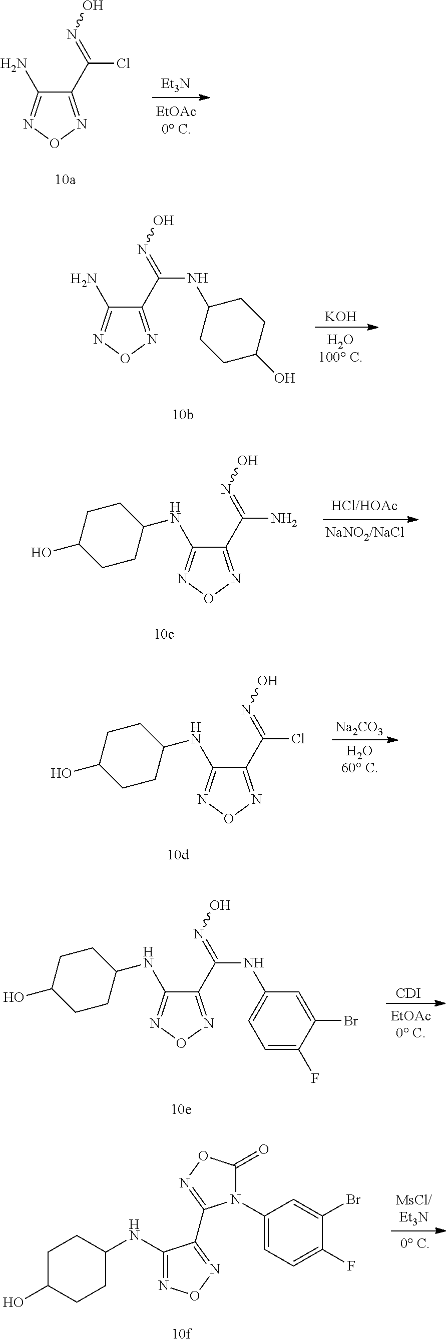

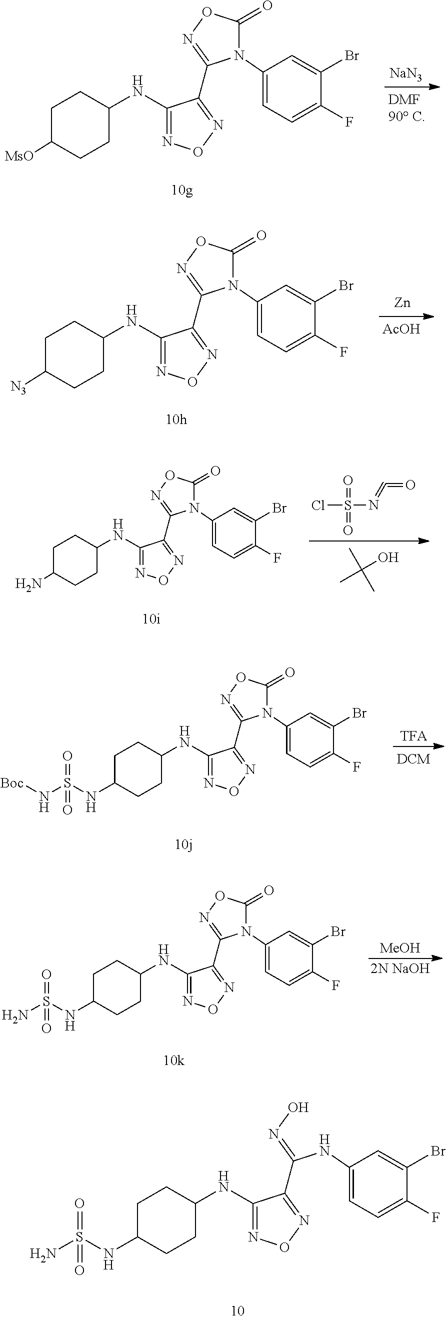

### Step 1: 4-amino-N′-hydroxy-N-(4-hydroxycyclohexyl)-1,2,5-oxadiazole-3-carboximidamide 10b

The compound 4-amino-N-hydroxy-1,2,5-oxadiazole-3-carboximidoyl chloride (9 g, 55 mmol) was dissolved in ethyl acetate (100 mL) at 0° C., and then 4-aminocyclohexane-1-ol (7.0 g, 61 mmol) was slowly added. The reaction solution was stirred at 0° C. for 30 minutes. Triethylamine (11.5 mL, 82.5 mmol) was slowly added, and the reaction solution was stirred at 0° C. for another 30 minutes. The reaction solution was added into water, and the organic phase was separated, washed with saturated brine, dried over anhydrous sodium sulfate and concentrated in vacuo to obtain a crude product. The crude product was recrystallized from dichloromethane (30 mL) to obtain the compound 4-amino-N′-hydroxy-N-(4-hydroxycyclohexyl)-1,2,5-oxadiazole-3-carboximidamide 10b (12 g), yield 89%.

¹H NMR (400 MHz, DMSO-d₆, ppm): δ 10.7 (s, 1H), 6.25-6.35 (br, 2H), 5.67 (d, 1H), 4.50 (d, 1H), 3.65-3.75 (m, 1H) 3.28-3.38 (m, 1H), 1.69-1.83 (m, 4H), 1.25-1.40 (m, 2H), 1.05-1.20 (m, 2H).

### Step 2: N¹-hydroxy-4-((4-hydroxycyclohexyl)amino)-1,2,5-oxadiazole-3-carboximidamide 10c

The compound 4-amino-N′-hydroxy-N-(4-hydroxycyclohexyl)-1,2,5-oxadiazole-3-carboximidamide 10b (12 g, 49.8 mmol) was suspended in water (60 mL), and then KOH (8.3 g, 0.15 mol) was slowly added. The reaction solution was heated to reflux for 48 hours and then cooled to room temperature. The mixture was extracted with ethyl acetate (50 mL×3) and washed with saturated brine. The organic phase was dried over anhydrous sodium sulfate and concentrated in vacuo to obtain N′-hydroxy-4-((4-hydroxycyclohexyl)amino)-1,2,5-oxadiazole-3-carboximidamide 10c (3.6 g), yield 30%.

¹H NMR (400 MHz, DMSO-d₆, ppm): δ 10.5 (s, 1H), 6.19-6.25 (br, 2H), 5.96 (d, 1H), 4.58 (d, 1H), 3.40-3.48 (m, 1H) 3.20-3.30 (m, 1H), 1.98-2.08 (m, 2H), 1.78-1.88 (m, 2H), 1.22-1.32 (m, 4H).

### Step 3: N-hydroxy-4-((4-hydroxycyclohexyl)amino)-1,2,5-oxadiazole-3-carbimidoyl chloride 10d

The compound N′-hydroxy-4-((4-hydroxycyclohexyl)amino)-1,2,5-oxadiazole-3-carboximidamide 10c (3.6 g, 14.9 mmol) was suspended in 6 N HCl (30 mL). The mixture was stirred constantly to obtain a clear solution, and then sodium chloride (2.62 g, 44.8 mmol) was added at 0° C. At 0° C., sodium nitrite (1.03 g, 14.9 mmol) in water (5 mL) was added slowly to the reaction solution, and the reaction solution was stirred at 0° C. for 2 hours. The reaction solution was filtered. A solid was collected and dried to obtain N-hydroxy-4-((4-hydroxycyclohexyl)amino)-1,2,5-oxadiazole-3-carbimidoyl chloride 10d (3.1 g)), yield 79%.

MS m/z (ESI): 259 (M−H).

### Step 4: N-(3-bromo-4-fluorophenyl)-N′-hydroxy-4-((4-hydroxycyclohexyl)amino)-1,2,5-oxadiazole-3-carboximidamide 10e

The compound N-hydroxy-4-((4-hydroxycyclohexyl)amino)-1,2,5-oxadiazole-3-N-hydroxy-4-((4-hydroxycyclohexyl)amino)-1,2,5-oxadiazole-3-carbimidoyl chloride 10d (2.4 g, 9.2 mmol) and 3-bromo-4-fluoroaniline (1.75 g, 9.2 mmol) were suspended in water (35 mL). The reaction mixture was heated to 60° C. for 5 minutes. Sodium bicarbonate (1.16 g, 13.8 mmol) was added to the reaction solution in one portion at 60° C. The reaction solution was stirred at 60° C. for 20 minutes and then cooled to room temperature. The reaction mixture was extracted with ethyl acetate (50 mL×3) and washed with saturated brine. The organic phase was dried over anhydrous sodium sulfate and concentrated in vacuo to obtain N-(3-bromo-4-fluorophenyl)-N′-hydroxy-4-N-(3-bromo-4-fluorophenyl)-N′-hydroxy-4-((4-hydroxycyclohexyl)amino)-1,2,5-zole-3-carboximidamide 10e (3.8 g).

MS m/z (ESI): 413 (M+H).

### Step 5: 4-(3-bromo-4-fluorophenyl)-3-(4-((4-hydroxycyclohexyl)amino)-1,2,5-oxadiazol-3-yl)-1,2,4-oxadiazol-5(4H)-one 10f

The compound N-(3-bromo-4-fluorophenyl)-N′-hydroxy-4-((4-hydroxycyclohexyl)amino)-1,2,5-oxadiazole-3-carboximidamide 10e (3.8 g, crude) was dissolved in ethyl acetate (40 mL), and then N,N-carbonyldiimidazole (1.47 g, 9.2 mmol) was added slowly at 0° C. The reaction solution was stirred at 0° C. for 2 hours and slowly risen to room temperature. The mixture was washed with saturated brine, dried over anhydrous sodium sulfate and concentrated in vacuo to obtain a crude product. The crude product was recrystallized from dichloromethane (30 mL) to obtain 4-(3-bromo-4-fluorophenyl)-3-(4-((4-hydroxycyclohexyl)amino)-1,2,5-oxadiazol-3-yl)-1,2,4-oxadiazol-5(4H)-one 10f (3.76 g), yield 92%.

MS m/z (ESI): 438 (M−H).

### Step 6: 4-((4-(4-(3-bromo-4-fluorophenyl)-5-oxo-4,5-dihydro-1,2,4-oxadiazol-3-yl)-1,2,5-oxadiazol-3-yl)amino)cyclohexyl methanesulfonate 10g

The compound 4-(3-bromo-4-fluorophenyl)-3-(4-((4-hydroxycyclohexyl)amino)-1,2,5-oxadiazol-3-yl)-1,2,4-oxadiazol-5(4H)-one 10f (1.36 g, 3.1 mmol) was dissolved in ethyl acetate (20 mL), and then methanesulfonyl chloride (0.36 mL, 4.63 mmol) was added at 0° C. The reaction solution was stirred at 0° C. for 5 minutes and then triethylamine (1.3 mL, 9.3 mmol) was slowly added. The reaction solution was stirred at 0° C. for 60 minutes and washed with saturated brine. The organic phase was dried over anhydrous sodium sulfate and concentrated in vacuo to obtain a crude product. The crude product was subjected to column chromatography (petroleum ether: ethyl acetate=1:1) to obtain 4-((4-(4-(3-bromo-4-fluorophenyl)-5-oxo-4,5-dihydro-1,2,4-oxadiazol-3-yl)-1,2,5-oxadiazol-3-yl)amino)cyclohexyl methanesulfonate 10 g (1.3 g), yield 85%.

¹H NMR (400 MHz, DMSO-d₆, ppm): 8.01-8.07 (m, 1H), 7.55-7.70 (m, 2H), 6.25 (d, 1H), 4.55-4.65 (m, 1H), 3.33-3.43 (m, 1H) 3.20 (s, 3H), 1.92-2.08 (m, 4H), 1.43-1.68 (m, 4H).

### Step 7: 3-(4-((4-azidocyclohexyl)amino)-1,2,5-oxadiazol-3-yl)-4-(3-bromo-4-fluorophenyl)-1,2,4-oxadiazol-5(4H)-one 10h

The compound 4-((4-(4-(3-bromo-4-fluorophenyl)-5-oxo-4,5-dihydro-1,2,4-4-((4-(4-(3-bromo-4-fluorophenyl)-5-oxo-4,5-dihydro-1,2,4-oxadiazol-3-yl)-1,2,5-azol-3-yl)amino)cyclohexyl methanesulfonate 10 g (0.9 g, 1.74 mmol) was dissolved in N,N-dimethylformamide (10 mL), and then sodium azide (340 mg, 5.21 mmol) was added. The reaction solution was heated to 90° C. and stirred for 60 minutes. TLC showed that the reaction was completed. The reaction mixture was concentrated to dryness in vacuo to obtain the crude product 3-(4-((4-azidocyclohexyl)amino)-1,2,5-3-(4-((4-azidocyclohexyl)amino)-1,2,5-oxadiazol-3-yl)-4-(3-bromo-4-fluorophenyl)-4-oxadiazol-5(4H)-one 10h (800 mg).

### Step 8: 3-(4-((4-aminocyclohexyl)amino)-1,2,5-oxadiazol-3-yl)-4-(3-bromo-4-fluorophenyl)-1,2,4-oxadiazol-5(4H)-one 10i

The compound 3-(4-((4-azidocyclohexyl)amino)-1,2,5-oxadiazol-3-yl)-4-(3-bromo-4-fluorophenyl)-1,2,4-oxadiazole-5(4H)-one 10h (350 mg) was dissolved in glacial acetic acid (10 mL), and then zinc powder (490 g, 7.5 mmol) was added. The reaction solution was stirred at room temperature for 2 hours and then concentrated to dryness in vacuo. Ethyl acetate (25 mL) was added. The mixture was washed with saturated aqueous sodium bicarbonate solution and saturated brine. The solid was filtered off. The organic phase was dried over anhydrous sodium sulfate and concentrated to dryness in vacuo to obtain a crude product. The crude product was subjected to column chromatography (dichloromethane:methanol=30:1) to obtain 3-(4-((4-aminocyclohexyl)amino)-1,2,5-oxadiazol-3-yl)-4-(3-bromo-4-fluorophenyl)-1,2,4-oxadiazol-5(4H)-one 10i (250 mg), yield 76%.

MS m/z (ESI): 439 (M+H).

### Step 9: tert-butyl (N-(4-((4-(4-(3-bromo-4-fluorophenyl)-5-oxo-4,5-dihydro-1,2,4-oxadiazol-3-yl)-1,2,5-oxadiazol-3-yl)amino)cyclohexyl)sulfamoyl)carbamate 10j

The compound chlorosulfonyl isocyanate (126 mg, 0.89 mmol) was dissolved in dichloromethane (5 mL), and then tert-butanol (65 mg, 0.89 mmol) was added at 0° C. The mixture was stirred for 20 minutes to obtain intermediate solution A. The compound 3-(4-((4-aminocyclohexyl)amino)-1,2,5-oxadiazol-3-yl)-4-(3-bromo-4-fluorophenyl)-1,2,4-oxadiazol-5(4H)-one 10i (260 mg, 0.59 mmol) was dissolved in dichloromethane (10 mL), followed by the addition of the intermediate solution A at 0° C. The mixture was stirred for 5 minutes, and then triethylamine (0.25 mL, 1.78 mmol) was added. The reaction mixture was stirred at 0° C. for 30 minutes, and then ethyl acetate (50 mL) was added. The mixture was washed with brine. The organic phase was dried over anhydrous sodium sulfate and concentrated to dryness in vacuo to obtain a crude product. The crude product was purified by column chromatography (petroleum ether: ethyl acetate=1:1) to obtain the compound tert-butyl (N-(4-((4-(4-(3-bromo-4-fluorophenyl)-5-oxo-4,5-dihydro-1,2,4-oxadiazol-3-yl)-1,2,5-oxadiazol-3-yl)amino)cyclohexyl)sulfamoyl)carbamate 10j (140 mg), yield 38%.

MS m/z (ESI): 616 (M−H).

### Step 10: 3-(4-((4-(sulfamoylamino)cyclohexyl)amino)-1,2,5-oxadiazol-3-yl)-4-(3-bromo-4-fluorophenyl)-1,2,4-oxadiazol-5(4H)-one 10k

The compound tert-butyl (N-(4-((4-(4-(3-bromo-4-fluorophenyl)-5-oxo-4,5-dihydro-1,2,4-oxadiazol-3-yl)-1,2,5-oxadiazol-3-yl)amino)cyclohexyl)sulfamoyl)carbamate 10j (120 mg) was dissolved in dichloromethane (3 mL) at 0° C., and then trifluoroacetic acid (3 mL) was added slowly. The reaction solution was stirred at 0° C. for 30 minutes and then concentrated to dryness in vacuo to obtain the crude product 3-(4-((4-(sulfamoylamino)cyclohexyl)amino)-1,2,5-oxadiazol-3-yl)-4-(3-bromo-4-fluorophenyl)-1,2,4-oxadiazol-5(4H)-one 10k (120 mg, a brown viscous material).

### Step 11: (Z)—N-(3-bromo-4-fluorophenyl)-N′-hydroxy-4-((4-(sulfamoylamino)cyclohexyl)amino)-1,2,5-oxadiazole-3-carboximidamide 10

The compound 3-(4-((4-(sulfamoylamino)cyclohexyl)amino)-1,2,5-oxadiazol-3-yl)-4-(3-bromo-4-fluorophenyl))-1,2,4-oxadiazol-5(4H)-one 10k (100 mg, crude) was dissolved in methanol (2 mL), and then sodium hydroxide (15 mg, 0.375 mmol, 2M aqueous solution) was added to the reaction solution. The reaction solution was stirred at room temperature for 30 minutes. The reaction solution was adjusted to pH 7 with 1N hydrochloric acid. The reaction solution was extracted with ethyl acetate and washed with saturated brine. The organic phase was dried over anhydrous sodium sulfate and concentrated in vacuo to obtain a crude product. The crude product was recrystallized from dichloromethane to obtain the compound (Z)—N-(3-bromo-4-fluorophenyl)-N′-hydroxy-4-((4-(sulfamoylamino)cyclohexyl)amino-1,2,5-oxadiazole-3-carboximidamide 10 (50 mg, white solid), yield 53%.

MS m/z (ESI): 490 (M−H).

¹H NMR (400 MHz, DMSO-d₆, ppm): 11.6 (s, 1H), 8.91 (s, 1H), 7.18-7.22 (m, 1H), 7.10-7.15 (m, 1H), 6.79-6.85 (m, 1H), 6.51 (s, 2H), 6.43 (d, 1H), 6.06 (d, 1H), 3.43-3.50 (m, 1H), 3.23-3.33 (m, 1H), 1.62-1.85 (m, 8H).

### Example 11

### (Z)—N-(3-bromo-4-fluorophenyl)-N′-hydroxy-4-((3-(sulfamoylamino)cyclobutyl)amino)-1,2,5-oxadiazole-3-carboximidamide (11)

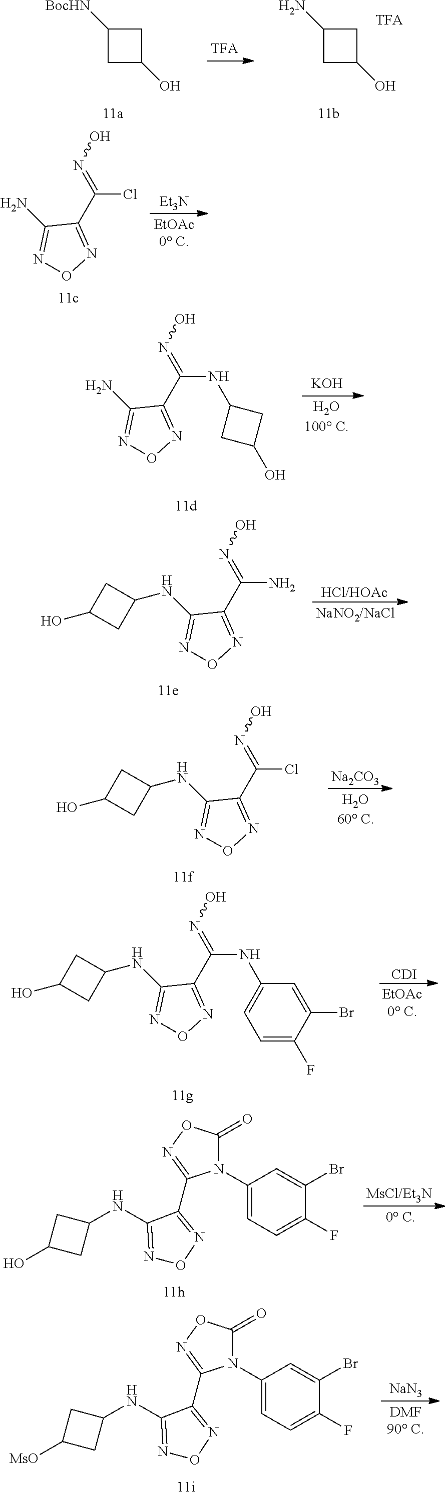

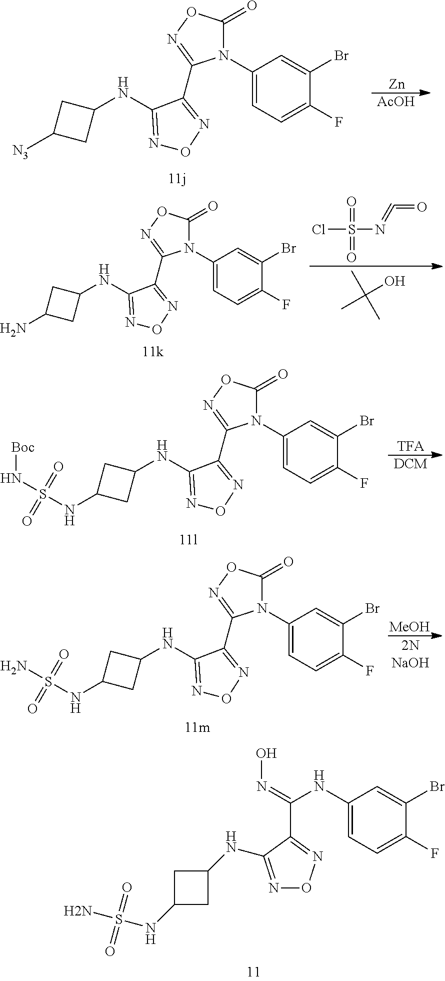

### Step 1: aminocyclobutan-1-ol trifluoroacetate 11b

Compound tert-butyl (3-hydroxycyclobutyl)carbamate 11a (9 g, 48 mmol) was dissolved in dichloromethane (20 mL), and then trifluoroacetic acid (20 mL) was slowly added at 0° C. The reaction mixture was stirred at room temperature for 3 hours and (9 g), yield 100

¹H NMR (400 MHz, MeOD, ppm): 5.43-5.49 (m, 1H), 4.43-4.49 (i, 0.7H), 3.99-4.06 (m, 1H), 3.84-3.91 (m, 0.7H), 2.67-2.78 (m, 4.3H), 2.33-2.47 (m, 3.4H).

### Step 2: 4-amino-N′-hydroxy-N-(3-hydroxycyclobutyl)-1,2,5-oxadiazole-3-carboximidamide 11d

The compound 3-aminocyclobutan-1-ol trifluoroacetate 11b (9 g, 48 mmol) was dissolved in ethyl acetate (25 mL), and then potassium carbonate (13.5 g, 97 ol) was slowly added. The reaction mixture was stirred at room temperature for 10 minutes. The solid was removed to obtain a free base compound 1b solution. The compound 4-amino-N-4-amino-N-hydroxy-1,2,5-oxadiazole-3-carboximidoyl chloride (6.6 g, 40 mmol) was dissolved in ethyl acetate (25 mL). The free base 3-aminocyclobutan-1-ol trifluoroacetate 1b solution was slowly added at 0° C. The reaction solution was stirred at 0° C. for 30 minutes, and then triethylamine (16.7 mL, 120 mmol) was slowly added. The reaction solution was stirred at 0° C. for 30 minutes, and then added in water. The organic phase was separated, washed with saturated brine, dried over anhydrous sodium sulfate and concentrated to dryness in vacuo to obtain a crude product. The crude product was purified by silica column chromatography (petroleum ether:ethyl acetate=1:1) to obtain 4-amino-N′-hydroxy-N-(3-hydroxycyclobutyl)-1,2,5-oxadiazole-3-4-amino-N′-hydroxy-N-(3-hydroxycyclobutyl)-1,2,5-oxadiazole-3-carboximidamide 11d (4.2 g), yield 49%.

¹H NMR (400 MHz, DMSO-d₆, ppm): δ 10.7 (s, 1H), 6.25-6.30 (m, 3H), 4.92 (d, 1H), 4.43-4.53 (m, 1H) 4.18-4.27 (m, 1H), 2.15-2.24 (m, 2H), 2.03-2.09 (m, 2H).

### Step 3: N¹-hydroxy-4-((3-hydroxycyclobutyl)amino)-1,2,5-oxadiazole-3-carboximidamide 11e

The compound 4-amino-N′-hydroxy-N-(3-hydroxycyclobutyl)-1,2,5-oxadiazole-3-carboximidamide 11d (4.2 g, 19.7 mmol) was suspended in water (20 mL), and then KOH (3.3 g, 59.1 mmol) was slowly added. The reaction solution was heated to reflux for 48 hours and then cooled to room temperature. The mixture was extracted with ethyl acetate (50 mL×3) and washed with saturated brine. The organic phase was dried over anhydrous sodium sulfate and concentrated to dryness in vacuo to obtain N′-hydroxy-4-((3-hydroxycyclobutyl)amino)-1,2,5-oxadiazole-3-carboximidamide 11e (2.2 g), yield 52%.

MS m/z (ESI): 212 (M−H).

### Step 4: N-hydroxy-4-((3-hydroxycyclobutyl)amino)-1,2,5-oxadiazole-3-carbimidoyl chloride 11f

The compound N′-hydroxy-4-((3-hydroxycyclobutyl)amino)-1,2,5-oxadiazole-3-carboximidamide 11e (1.8 g, 8.4 mmol) was suspended in 6N HCl (30 mL). The suspension was stirred continually to obtain a clear solution. Sodium chloride (1.46 g, 25.2 mmol) was added to the above solution at 0° C., followed by addition of a solution of sodium nitrite (0.58 g, 8.4 mmol) in water (2 mL). The reaction solution was stirred at 0° C. for 2 hours. The mixture was extracted with ethyl acetate (50 mL×3) and washed with saturated brine. The organic phase was dried over anhydrous sodium sulfate and concentrated to dryness in vacuo to obtain N-hydroxy-4-((3-hydroxycyclobutyl)amino)-1,2,5-oxadiazole-3-carbimidoyl chloride 11f (1.95 g), yield 100%.

MS m/z (ESI): 231(M−H).

### Step 5: N-(3-bromo-4-fluorophenyl)-N′-hydroxy-4-((3-hydroxycyclobutyl)amino)-1,2,5-oxadiazole-3-carboximidamide 112

The compound N-hydroxy-4-((3-hydroxycyclobutyl)amino)-1,2,5-oxadiazole-3-carbimidoyl chloride 11f (1.95 g, 8.4 mmol) and 3-bromo-4-fluoroaniline (1.59 g, 8.4 mmol) were suspended in water (25 mL), and then the mixture was heated to 60° C. for 5 minutes. Sodium bicarbonate (1.06 g, 12.6 mmol) was added in one portion to the reaction solution at 60° C. The reaction solution was stirred at 60° C. for 20 minutes and then cooled to room temperature. The mixture was extracted with ethyl acetate (50 mL×3) and washed with saturated brine. The organic phase was dried over anhydrous sodium sulfate and concentrated to dryness in vacuo to obtain N-(3-bromo-4-fluorophenyl)-N′-hydroxy-4-((3-hydroxycyclobutyl)amino)-1,2,5-oxadiazole-3-carboximidamide 11 g (3.24 g, crude).

### Step 6: 4-(3-bromo-4-fluorophenyl)-3-(4-((3-hydroxycyclobutyl)amino)-1,2,5-oxadiazol-3-yl)-1,2,4-oxadiazol-5(4H)-one 11h

The compound N-(3-bromo-4-fluorophenyl)-N′-hydroxy-4-((3-hydroxycyclobutyl)amino)-1,2,5-oxadiazole-3-carboximidamide 11 g (3.24 g, crude) was dissolved in ethyl acetate (20 mL), and then N,N-carbonyldiimidazole (1.36 g, 8.4 mmol) was slowly added at 0° C. The mixture was stirred at 0° C. for 2 hours and risen slowly to room temperature. The mixture was washed with saturated brine. The organic phase was dried over anhydrous sodium sulfate and concentrated to dryness in vacuo to obtain a crude product. The crude product was purified by column chromatography (petroleum ether: ethyl acetate=1:1) to obtain 4-(3-bromo-4-fluorophenyl)-3-(4-((3-hydroxycyclobutyl)amino)-1,2,5-oxadiazol-3-yl)-1,2,4-oxadiazol-5(4H)-one 11h (1.66 g), yield 47%.

¹H NMR (400 MHz, DMSO-d₆, ppm): δ 8.02-8.05 (m, 1H), 7.65-7.69 (m, 1H), 7.57-7.62 (m, 1H), 6.60 (d, 1H), 5.07 (d, 1H), 4.20-4.28 (m, 1H) 3.96-4.06 (m, 1H), 2.21-2.25 (m, 2H), 2.10-2.16 (n, 2H).

### Step 7: 3-((4-(4-(3-bromo-4-fluorophenyl)-5-oxo-4,5-dihydro-1,2,4-oxadiazol-3-yl)-1,2,5-oxadiazol-3-yl)amino)cyclobutyl methanesulfonate 11i

The compound 4-(3-bromo-4-fluorophenyl)-3-(4-((3-hydroxycyclobutyl)amino)-4-(3-bromo-4-fluorophenyl)-3-(4-((3-hydroxycyclobutyl)amino)-1,2,5-oxadiazol-3-yl)-1,2,4-oxadiazol-5(4H)-one 1h (0.5 g, 1.2 mmol) was dissolved in ethyl acetate (10 mL), and methanesulfonyl chloride (0.14 mL, 1.8 mmol) was added at 0° C. The reaction solution was stirred at 0° C. for 5 minutes, and then triethylamine (0.51 mL, 3.6 mmol) was added slowly. The reaction solution was stirred at 0° C. for 60 minutes and washed with saturated brine. The organic phase was dried over anhydrous sodium sulfate and concentrated to dryness in vacuo to obtain a crude product. The crude product was purified by column chromatography (petroleum ether: ethyl acetate=1:1) to obtain 3-3-((4-(4-(3-bromo-4-fluorophenyl)-5-oxo-4,5-dihydro-1,2,4-oxadiazol-3-yl)-1,2,5-azol-3-yl)amino)cyclobutyl methanesulfonate 11i (0.56 g), yield 94%.

¹H NMR (400 MHz, DMSO-d₆, ppm): δ 8.07-8.09 (m, 1H), 7.69-7.73 (m, 1H), 7.57-7.62 (m, 1H), 6.86 (d, 1H), 5.14-5.19 (m, 1H) 4.14-4.19 (m, 1H), 3.18 (s, 3H), 2.57-2.61 (m, 4H).

### Step 8: 3-(4-((3-azidocyclobutyl)amino)-1,2,5-oxadiazol-3-yl)-4-(3-bromo-4-fluorophenyl)-1,2,4-oxadiazol-5(4H)-one 11i

The compound 3-((4-(4-(3-bromo-4-fluorophenyl)-5-oxo-4,5-dihydro-1,2,4-oxadiazol-3-yl)-1,2,5-oxadiazol-3-yl)amino)cyclobutyl methanesulfonate 11i (504 mg, 1.03 mmol) was dissolved in N,N-dimethylformamide (5 mL), and then sodium azide was added (198 mg, 3.09 mmol). The reaction solution was heated to 90° C. and stirred for 60 minutes. TLC showed that the reaction was completed, and then the mixture was concentrated to dryness in vacuo to obtain the crude product 3-(4-((3-azidocyclobutyl)amino)-1,2,5-oxadiazol-3-yl)-4-(3-bromo-4-fluorophenyl)-1,2,4-oxadiazol-5(4H)-one 11j (450 mg).

MS m/z (ESI): 435 (M−H).

### Step 9: 3-(4-((3-aminocyclobutyl)amino)-1,2,5-oxadiazol-3-yl)-4-(3-bromo-4-fluorophenyl)-1,2,4-oxadiazol-5(4H)-one 11k

The compound 3-(4-((3-azidocyclobutyl)amino)-1,2,5-oxadiazol-3-yl)-4-(3-bromo-4-fluorophenyl)-1,2,4-oxadiazole-5(4H)-one 11j (450 mg) was dissolved in glacial acetic acid (10 mL), and then zinc powder (670 g, 10.3 mmol) was added. The mixture was stirred at room temperature for 2 hours. The reaction solution was concentrated to dryness in vacuo, and then ethyl acetate (25 mL) was added. The mixture was washed with saturated aqueous sodium bicarbonate solution and saturated brine. The solid was filtered off. The organic phase was dried over anhydrous sodium sulfate and concentrated to dryness in vacuo to obtain a crude product. The crude product was purified by column chromatography (dichloromethane:methanol=30:1) to obtain 3-(4-((3-aminocyclobutyl)amino)-1,2,5-oxadiazol-3-yl)-4-(3-bromo-4-fluorophenyl)-1,2,4-oxadiazol-5(4H)-one 11k (350 mg), yield 82%.

MS m/z (ESI): 409 (M−H).

### Step 10: tert-butyl (N-(3-((4-(4-(3-bromo-4-fluorophenyl)-5-oxo-4,5-dihydro-1,2,4-oxadiazol-3-yl)-1,2,5-oxadiazol-3-yl)amino)cyclobutyl)sulfamoyl)carbamate 11l

The compound chlorosulfonyl isocyanate (102 mg, 0.72 mmol) was dissolved in dichloromethane (5 mL), and then tert-butanol (54 mg, 0.72 mmol) was added at 0° C. The mixture was stirred for 20 minutes to obtain intermediate solution A. The compound 3-(4-((3-aminocyclobutyl)amino)-1,2,5-oxadiazol-3-yl)-4-(3-bromo-4-fluorophenyl)-1,2,4-oxadiazol-5(4H)-one 11k (200 mg, 0.48 mmol) was dissolved in dichloromethane (10 mL). Intermediate solution A was added at 0° C., and then the reaction mixture was stirred for 5 minutes, followed by addition of triethylamine (0.20 mL, 1.44 mmol). The reaction mixture was stirred at 0° C. for 30 minutes. Ethyl acetate (50 mL) was added, and the mixture was washed with saturated brine. The organic phase was dried over anhydrous sodium sulfate and concentrated to dryness in vacuo to obtain a crude product. The crude product was purified by column chromatography (petroleum ether: ethyl acetate=1:1) to obtain the compound tert-butyl (N-(3-((4-(4-(3-bromo-4-fluorophenyl)-5-oxo-4,5-dihydro-1,2,4-oxadiazol-3-yl)-1,2,5-oxadiazol-3-yl)amino)cyclobutyl)sulfamoyl)carbamate 11l (110 mg), yield 26%.

MS m/z (ESI): 588 (M−H).

### Step 11: 3-(4-((3-(sulfamoylamino)cyclobutyl)amino)-1,2,5-oxadiazol-3-yl)-4-(3-bromo-4-fluorophenyl)-1,2,4-oxadiazol-5(4H)-one 11m

The compound tert-butyl (N-(3-((4-(4-(3-bromo-4-fluorophenyl)-5-oxo-4,5-dihydro-1,2,4-oxadiazol-3-yl)-1,2,5-oxadiazol-3-yl)amino)cyclobutyl)sulfamoyl)carbamate 11l (110 mg) was dissolved in dichloromethane (3 mL), and then trifluoroacetic acid (3 mL) was slowly added at 0° C. The reaction solution was stirred at 0° C. for 30 minutes and then concentrated to dryness in vacuo to obtain the crude product 3-(4-((3-(sulfamoylamino)cyclobutyl)amino)-1,2,5-oxadiazol-3-yl)-4-(3-bromo-4-fluorophenyl)-1,2,4-oxadiazol-5(4H)-one 11m (90 mg) MS m/z (ESI): 488 (M−H).

### Step 12: (Z)—N-(3-bromo-4-fluorophenyl)-N′-hydroxy-4-((3-(sulfamoylamino)cyclobutyl)amino)-1,2,5-oxadiazole-3-carboximidamide 11

The compound 3-(4-((3-(sulfamoylamino)cyclobutyl)amino)-1,2,5-oxadiazol-3-3-(4-((3-(sulfamoylamino)cyclobutyl)amino)-1,2,5-oxadiazol-3-yl)-4-(3-bromo-4-phenyl)-1,2,4-oxadiazol-5(4H)-one 1 m (90 mg, crude) was dissolved in methanol (2 mL), and then sodium hydroxide (15 mg, 0.375 mmol, 2 M aqueous solution) was added to the reaction solution. The reaction solution was stirred at room temperature for 30 minutes and then adjusted to pH 7 with 1N HCl. The reaction solution was extracted with ethyl acetate and washed with saturated brine. The organic phase was dried over anhydrous sodium sulfate and concentrated in vacuo to obtain a crude product. The crude product was recrystallized from dichloromethane (1 mL) to obtain ((Z)—N-(3-((Z)—N-(3-bromo-4-fluorophenyl)-N′-hydroxy-4-((3-(sulfamoylamino)cyclobutyl)amino)-1,2,5-oxadiazole-3-carboximidamide 11 (11 mg), yield 13%.

MS m/z (ESI): 462 (M−H).

¹H NMR (400 MHz, MeOD, ppm): δ 7.01-7.03 (m, 1H), 6.93-6.97 (m, 1H), 6.72-6.76 (m, 1H), 3.62-3.70 (m, 1H) 3.48-3.57 (m, 1H), 2.71-2.79 (m, 2H), 1.83-1.93 (m, 2H).

### Example 12

tert-butyl (Z)—(N-(4-((4-(N-(3-bromo-4-fluorophenyl)-N′-hydroxycarbamimidoyl)-1,2,5-oxadiazol-3-yl)amino)cyclohexyl)sulfamoyl)carbamate (12)

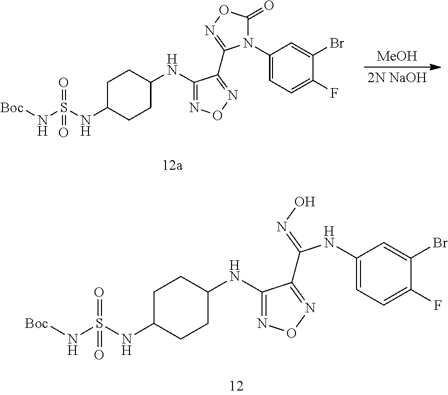

The compound tert-butyl (N-(4-((4-(4-(3-bromo-4-fluorophenyl)-5-oxo-4,5-dihydro-1,2,4-oxadiazol-3-yl)-1,2,5-oxadiazol-3-yl)amino)cyclohexyl)sulfamoyl)carbamate 12a (17 mg, 0.027 mmol) was dissolved in methanol (0.3 mL). Sodium hydroxide (2.2 mg, 0.054 mmol, 2 M aqueous solution) was added to the reaction solution. The reaction solution was stirred at room temperature for 30 minutes and concentrated in vacuo to obtain a crude product. The crude product was washed with water to obtain tert-butyl (Z)—(N-(4-((4-(N-(3-bromo-4-fluorophenyl)-N′-hydroxycarbamimidoyl)-1,2,5-oxadiazol-3-yl)amino)cyclohexyl)sulfamoyl)carbamate 12 (11 mg), yield 69%.

MS m/z (ESI): 590 (M−H).

¹H NMR (400 MHz, DMSO-d₆, ppm): 11.6 (s, 1H), 8.91 (s, 1H), 7.18-7.22 (m, 1H), 7.10-7.15 (m, 1H), 6.79-6.85 (m, 1H), 6.02-6.12 (m, 1H), 3.43-3.50 (m, 1H), 3.23-3.33 (m, 1H), 1.62-1.85 (m, 8H), 1.36 (s, 9H).

### Example 13

### (Z)—N-(3-bromo-4-fluorophenyl)-N′-hydroxy-4-{[12-({S-methyl-N-[(4-methylphenyl)sulfonyl]sulfonimidoyl}amino)ethyl]amino}-1,2,5-oxadiazole-3-carboximidamide (13)

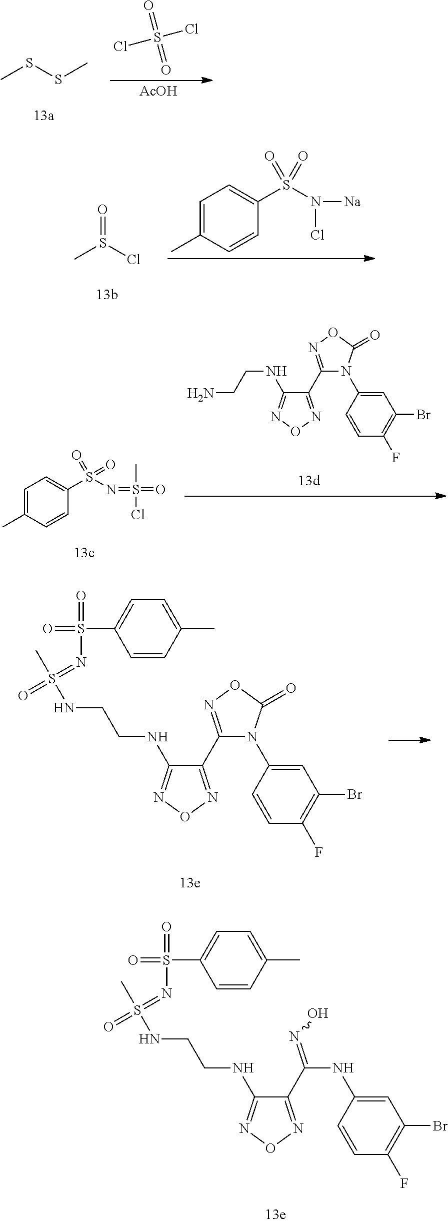

### Step 1: Methanesulfinic Chloride 13b

The compound 1,2-dimethyldisulfane (3.4 g, 36 mmol) was dissolved in acetic acid (4.34 g, 72 mmol), and then sulfonyl chloride (14.6 g, 108 mmol) was slowly added dropwise at −20° C. The reaction solution was stirred at −20° C. for 30 minutes, and slowly warmed up to room temperature and stirred for 2 hours, and then at 35° C. for another 1 hour. The mixture was concentrated in vacuo to remove volatile components and to obtain methanesulfinic chloride 13b (6 g), yield 48%.

### Step 2: N-tosylmethanesulfonimidoyl Chloride 13c

The compound chloramine T (1.5 g, 6.7 mmol) was added to toluene (50 mL). The mixture was heated to reflux for 5 hours, while water was removed by a water separator. The mixture was cooled to room temperature. Methanesulfinic chloride 1b (1 g, 10 mmol) was added to the reaction solution. The mixture was heated to 80° C. for 2 hours. After cooling to room temperature, the solid was removed. The reaction solution was concentrated in vacuo to obtain N-tosylmethanesulfonimidoyl chloride 13c (1.5 g), yield 79%.

¹H NMR (400 MHz, CDCl₃, ppm): δ 7.88 (d, 2H), 7.33 (d, 2H), 3.78 (s, 3H), 2.45 (s, 3H).

### Step 3: N-(((2-((4-(4-(3-bromo-4-fluorophenyl)-5-oxo-4,5-dihydro-1,2,4-oxadiazol-3-yl)-1,2,5-oxadiazol-3-yl)amino)ethyl)amino)(methyl)(oxo)-16-sulfanylidene)-4-methylbenzenesulfonamide 13e

The compound N-tosylmethanesulfonimidoyl chloride (175 mg, 0.65 mmol) was dissolved in tetrahydrofuran (10 mL), and then 3-(4-((2-aminoethyl)amino))-1,2,5-3-(4-((2-aminoethyl)amino))-1,2,5-oxadiazol-3-yl)-4-(3-bromo-4-fluorophenyl)-1,2,4-xadiazol-5(4H)-one (402 mg, 1.05 mmol) was added slowly at 0° C. The reaction solution was stirred at 0° C. for 30 minutes, and then added into water and extracted with ethyl acetate (15 mL×3). The organic phase was dried over anhydrous sodium sulfate and concentrated to dryness in vacuo to obtain a crude product. The crude product was purified by preparative thin-layer plate (dichloromethane:methanol=15:1) to obtain N—N-(((2-((4-(4-(3-bromo-4-fluorophenyl)-5-oxo-4,5-dihydro-1,2,4-oxadiazol-3-yl)-1,2,5-oxadiazol-3-yl)amino)ethyl)amino)(methyl)(oxo)-16-sulfanylidene)-4-fonamide 13e (140 mg), yield 37%.

¹H NMR (400 MHz, DMSO-d₆, ppm): δ 8.08 (m, 1H), 7.98-8.02 (br, 1H), 7.69 (d, 2H), 7.60 (m, 1H) 7.32 (d, 2H), 7.26 (br, 1H), 6.6 (m, 1H), 3.37-3.44 (m, 2H), 3.22-3.28 (m, 5H), 2.36 (s, 3H).

### Step 4: (Z)—N-(3-bromo-4-fluorophenyl)-N′-hydroxy-4-{[2-({S-methyl-N-[(4-methylphenyl)sulfonyl]sulfonimidoyl}amino)ethyl]amino}-1,2,5-oxadiazole-3-carboximidamide 13

The compound N-(((2-((4-(4-(3-bromo-4-fluorophenyl)-5-oxo-4,5-dihydro-1,2,4-oxadiazol-3-yl)-1,2,5-oxadiazol-3-yl)amino)ethyl)amino)(methyl)(oxo)-16-sulfanylidene)-4-methylbenzenesulfonamide 13e (35 mg, 0.056 mmol) was dissolved in methanol (1 mL), and then sodium hydroxide (5 mg, 0.114 mmol, 2 M aqueous solution) was added to the reaction solution. The mixture was stirred at room temperature for 1 hour. The reaction solution was adjusted to pH 8 with 1N hydrochloric acid, and then extracted with ethyl acetate and washed with saturated brine. The organic phase was dried over anhydrous sodium sulfate and concentrated in vacuo to obtain a crude product. The crude product was recrystallized from dichloromethane to obtain compound (Z)—N-(3-bromo-4-fluorophenyl)-N′-hydroxy-4-{[2-({S-methyl-N-[(4-methylphenyl)sulfonyl]sulfonimidoyl}amino)ethyl]amino}-1,2,5-oxadiazole-3-carboximidamide 13 (28 mg), yield 84%.

MS m/z (ESI): 590.0.

¹H NMR (400 MHz, DMSO-d₆, ppm): δ 11.5 (s, 1H), 8.92 (s, 1H), 8.02 (m, 1H), 7.69 (d, 2H), 7.32 (d, 2H), 7.14 (m, 1H), 7.09 (m 1H), 6.74 (m, 1H), 3.37-3.44 (m, 2H), 3.22-3.28 (m, 5H), 2.36 (s, 3H).

### Example 14

### (Z)—N-(3-bromo-4-fluorophenyl)-N′-hydroxy-4-((2-(S-methyl-N-(methylsulfonyl)sulfonimidoyl)ethyl)amino)-1,2,5-oxadiazole-3-carboximidamide

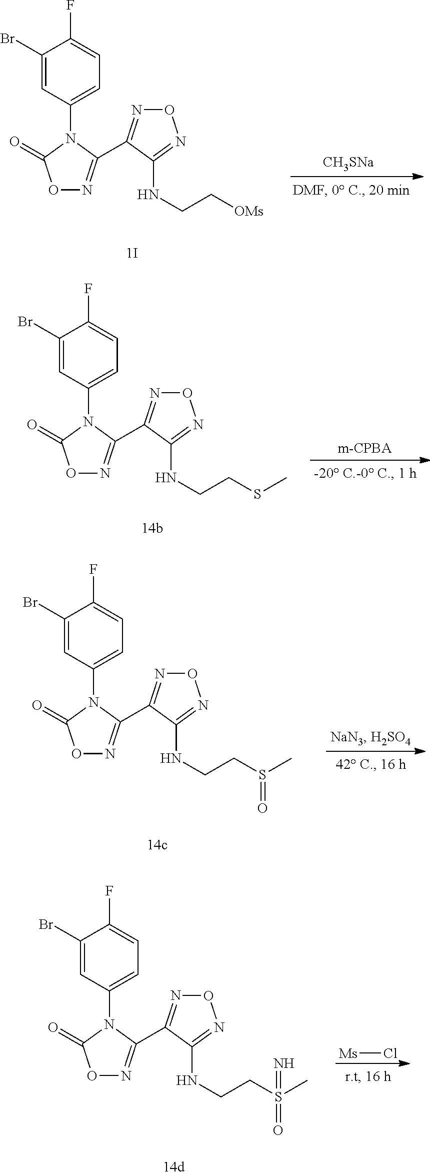 [SMILES: CS(=O)(=O)OCCNc1nonc1-c1noc(=O)n1-c1ccc(F)c(Br)c1 ; "CH3SNa" ; CSCCNc1nonc1-c1noc(=O)n1-c1ccc(F)c(Br)c1 ; CS(=O)CCNc1nonc1-c1noc(=O)n1-c1ccc(F)c(Br)c1 ; CS(=N)(=O)CCNc1nonc1-c1noc(=O)n1-c1ccc(F)c(Br)c1 ; CS(=O)(=O)Cl]

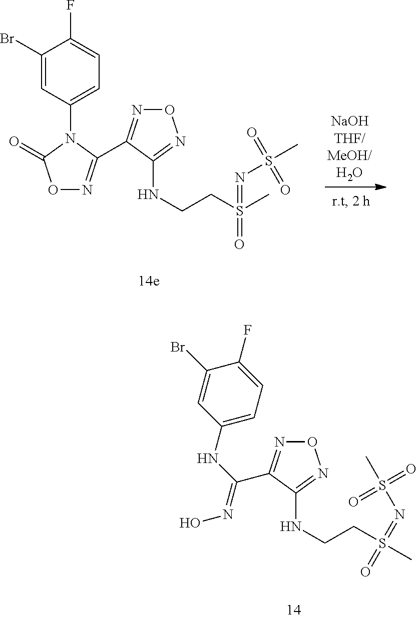

### Step 1: 4-(3-bromo-4-fluorophenyl)-3-(4-((2-(methylthio)ethyl)amino)-1,2,5-oxadiazol-3-yl)-1,2,4-oxadiazol-5(4H)-one 14b

In a 100 mL one-necked flask, 2-((4-(4-(3-bromo-4-fluorophenyl)-5-oxo-4,5-dihydro-1,2,4-oxadiazol-3-yl)-1,2,5-oxadiazol-3-yl)amino)ethyl methanesulfonate (2.6 g, 5.60 mmol) was dissolved in N,N-dimethylformamide (25 mL). The reaction mixture was cooled to 0° C. in an ice bath, and then sodium thiomethoxide (43.1 mg, 6.16 mmol) was added. The reaction mixture was stirred in an ice bath for 20 minutes, and monitored by LC-MS. After the raw material was completely converted, the reaction was stopped. The reaction was quenched by addition of water (50 mL), and then the mixture was extracted with ethyl acetate (50 mL×2). The combined organic phases were washed with saturated sodium chloride (50 mL), dried over anhydrous sulfuric acid and filtered. The filtrate was added with silica gel and dried, then directly purified by column chromatography (petroleum ether/ethyl acetate (5/1 to 3/1)) to obtain 4-(3-bromo-4-fluorophenyl)-3-(4-((2-(methylthio)ethyl)amino)-1,2,5-oxadiazol-3-yl)-1,2,4-oxadiazol-5(4H)-one 14b (1.0 g), yield 42.8%.

MS m/z (ESI): 416.0, 418.0 (M, M+2).

¹H NMR (400 MHz, CDCl₃, ppm) δ 7.62 (dd, J₁=5.6 Hz, J₂=2.4 Hz, 1H), 7.33 (m, 2H), 5.68 (t, J=5.2 Hz, 1H), 3.60 (dd, J₁=12.8 Hz, J₂=6.4 Hz, 2H), 2.80 (t, J=6.4 Hz, 2H), 2.15 (s, 3H).

### Step 2: 4-(3-bromo-4-fluorophenyl)-3-(4-((2-(methylsulfinyl)ethyl)amino)-1,2,5-oxadiazol-3-yl)-1,2,4-oxadiazol-5(4H)-one 14c

In a 100 mL one-necked flask, 4-(3-bromo-4-fluorophenyl)-3-(4-((2-(methylthio) ethyl)amino)-1,2,5-oxadiazol-3-yl)-1,2,4-oxadiazol-5(4H)-one (1.0 g, 2.41 mmol) was dissolved in dichloromethane (30 mL), and then the mixture was cooled to −40° C. in a dry ice-acetone bath. m-Chloroperoxybenzoic acid (457 mg, 2.65 mmol) dissolved in 5 mL of dichloromethane was added to the above solution dropwise. After the addition was completed, the dry ice acetone bath was removed. After about 20 minutes, the temperature slowly rose to room temperature. The reaction mixture was continually stirred at room temperature for 40 minutes and monitored by LC-MS. After the raw material was completely converted, the reaction was stopped. Water (50 mL) was and the mixture was extracted with ethyl acetate (50 mL×2). The combined organic phases were washed with saturated sodium chloride (60 mL), dried over anhydrous sodium sulfate and filtrated. The filtrate was concentrated to obtain 4-(3-bromo-4-4-(3-bromo-4-fluorophenyl)-3-(4-((2-(methylsulfinyl)ethyl)amino)-1,2,5-oxadiazol-3-)-1,2,4-oxadiazol-5(4H)-one 14c (0.9 g), yield 90%.

MS m/z (ESI): 432.0, 434.0 (M, M+2).

### Step 3: 4-(3-bromo-4-fluorophenyl)-3-(4-((2-(S-methylsulfonimidoyl)ethyl)amino)-1,2,5-oxadiazol-3-yl)-1,2,4-oxadiazol-5(4H)-one 14d

In a 100 mL one-necked flask, 4-(3-bromo-4-fluorophenyl)-3-(4-((2-(methylsulfinyl)ethyl)amino)-1,2,5-oxadiazol-3-yl)-1,2,4-oxadiazol-5(4H)-one (0.9 g, 2.08 mmol) was dissolved in chloroform (30 mL), and then sodium azide (275.0 mg, 4.16 mmol) was added. The mixture was cooled to 0° C. in an ice bath, and then concentrated sulfuric acid (0.5 mL) was added. Then the ice bath was removed, and the reaction mixture was heated to 42° C. in an oil bath. The reaction mixture was stirred overnight and monitored by LC-MS. After the raw material was completely converted, the reaction was stopped. Saturated sodium bicarbonate solution (50 mL) was added, and the mixture was extracted with ethyl acetate (50 mL×2). The combined organic phases were washed with saturated sodium chloride (50 mL), dried over sodium sulfate and filtered. The filtrate was concentrated to obtain 4-(3-bromo-4-fluorophenyl)-3-(4-((2-(S-methylsulfonimidoyl)ethyl)amino)-1,2,5-oxadiazol-3-yl)-1,2,4-oxadiazol-5(4H)-one 14d (0.7 g), yield 75.3%.

MS m/z (ESI): 447.0, 449.0 (M, M+2).

### Step 4: N-((2-((4-(4-(3-bromo-4-fluorophenyl)-5-oxo-4,5-dihydro-1,2,4-oxadiazol-3-yl)-1,2,5-oxadiazol-3-yl)amino)ethyl)(methyl)(oxo)-16-sulfanylidene)methanesulfonamide 14e

In a 100 mL one-necked flask, 4-(3-bromo-4-fluorophenyl)-3-(4-((2-(S-methylsulfonyliminoyl)ethyl)amino)-1,2,5-oxadiazol-3-yl)-1,2,4-oxadiazol-5(4H)-one (1.5 g, 3.36 mmol) was dissolved in dichloromethane (30 mL), and then methanesulfonyl chloride (1 mL, 10 mmol) was added. The mixture was stirred at room temperature for 15 minutes, and then triethylamine (1.5 mL, 10 mmol) was added. The reaction mixture was stirred overnight and monitored by LC-MS. After the raw material was completely converted, the reaction was stopped. Saturated sodium bicarbonate solution (50 mL) was added, and the reaction mixture was extracted with ethyl acetate (50 mL×2). The combined organic phases were washed with saturated sodium chloride (50 mL), dried over sodium sulfate and filtered. The filtrate was added with silica gel and dried, then directly purified by column chromatography with ethyl acetate/methanol (30/1 to 20/1) to obtain N-((2-((4-(4-(3-bromo-4-fluorophenyl)-5-oxo-4,5-dihydro-N-((2-((4-(4-(3-bromo-4-fluorophenyl)-5-oxo-4,5-dihydro-1,2,4-oxadiazol-3-yl)-1,2,5-xadiazol-3-yl)amino)ethyl)(methyl)(oxo)-16-sulfanylidene)methanesulfonamide 14e (0.65 g), yield 36.8%.

MS m/z (ESI): 525.0, 527.0 (M, M+2).

### Step 5: (Z)—N-(3-bromo-4-fluorophenyl)-N′-hydroxy-4-((2-(S-methyl-N-(methylsulfonyl)sulfonimidoyl)ethyl)amino)-1,2,5-oxadiazole-3-carboximidamide 14

In a 100 mL one-necked flask, N-((2-((4-(4-(3-bromo-4-fluorophenyl)-5-oxo-4,5-dihydro-1,2,4-oxadiazol-3-yl)-1,2,5-oxadiazol-3-yl)amino)ethyl)(methyl)(oxo)-16-sulfanylidene)methanesulfonamide (0.65 g, 1.24 mmol) was dissolved in tetrahydrofuran/methanol (8 mL/8 mL), and then sodium hydroxide (250 mg, 6.20 mmol) dissolved in water (4 mL) was added to the above solution. The mixture was stirred at room temperature for 2 hours and monitored by LC-MS. After the raw material was completely converted, the reaction was stopped. Saturated ammonium chloride solution (30 mL) was added, and the mixture was extracted with ethyl acetate (30 mL×2). The combined organic phases were washed with saturated sodium chloride (50 mL), dried over sodium sulfate and filtered. The filtrate was added with silica gel and dried, then directly purified by column chromatography with ethyl acetate/methanol (30/1 to 10/1) to obtain (Z)—N-(3-bromo-4-fluorophenyl)-N′-Hydroxy-4-((2-(S-methylsulfonyliminoyl)ethyl)amino)-1,2,5-oxadiazole-3-carboximidamide 14 (345 mg), yield 55.0%.

MS m/z (ESI): 499.0, 501.0 (M, M+2).

¹HNMR (400 MHz, DMSO-d₆, ppm) δ 11.45 (s, 1H), 8.92 (s, 1H), 7.18 (t, J=8.8 Hz, 1H), 7.12 (dd, J₁=6.0 Hz, J₂=2.8 Hz, 1H), 6.77 (m, 1H), 6.57 (t, J=6.0 Hz, 1H), 3.92 (m, 1H), 3.80 (m, 3H), 3.48 (s, 3H), 3.01 (s, 3H).

### Example 15

### (Z)—N-(3-bromo-4-fluorophenyl)-4-((2-(N-(cyclopropylsulfonyl)-S-methylsulfonimidoyl)ethyl)amino)-N′-hydroxy-1,2,5-oxadiazole-3-carboximidamide (15)

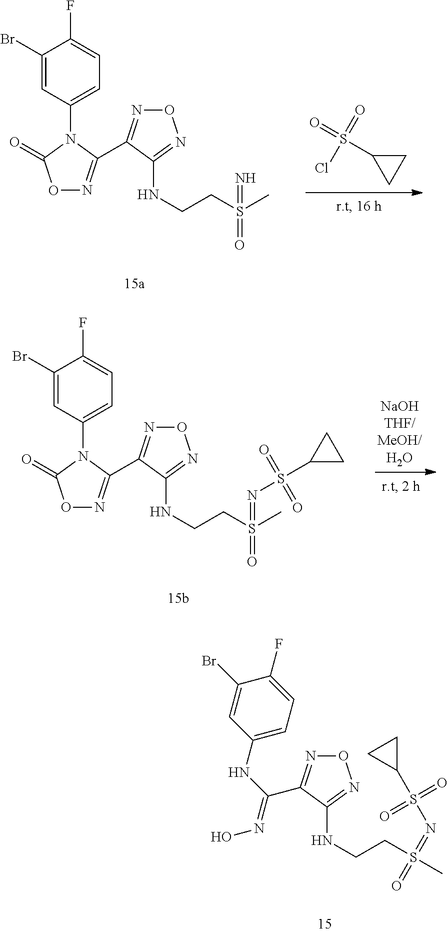 [SMILES: CS(=N)(=O)CCNc1nonc1-c1noc(=O)n1-c1ccc(F)c(Br)c1 ; O=S(=O)(Cl)C1CC1 ; CS(=O)(CCNc1nonc1-c1noc(=O)n1-c1ccc(F)c(Br)c1)=NS(=O)(=O)C1CC1 ; CS(=O)(CCNc1nonc1/C(=N/O)Nc1ccc(F)c(Br)c1)=NS(=O)(=O)C1CC1]

### Step 1: N-((2-((4-(4-(3-bromo-4-fluorophenyl)-5-oxo-4,5-dihydro-1,2,4-oxadiazol-3-yl)-1,2,5-oxadiazol-3-yl)amino)ethyl)(methyl)(oxo)-16-sulfanylidene)cyclopropanesulfonamide 15b

In a 100 mL one-necked flask, 4-(3-bromo-4-fluorophenyl)-3-(4-((2-(S-methyl sulfonimidyl)ethyl)amino)-1,2,5-oxadiazol-3-yl)-1,2,4-oxadiazol-5(4H)-one (1.5 g, 3.36 mmol) was dissolved in pyridine (30 mL), and then cyclopropylsulfonyl chloride (1.42 g, 10 mmol) and DMAP (41 mg, 3.36 mmol) were added. The reaction mixture was stirred overnight at room temperature. After the reaction was stopped, saturated sodium bicarbonate solution (50 mL) was added. The reaction mixture was extracted with ethyl acetate (50 mL×2). The combined organic phases were washed with saturated sodium chloride (50 mL), dried over anhydrous sodium sulfate and filtered. The filtrate was added with silica gel and dried, then directly purified by column chromatography with ethyl acetate/methanol (30/1 to 20/1) to obtain N-((2-((4-(4-(3-bromo-4-fluorophenyl)-5-oxo-4,5-dihydro-1,2,4-oxadiazol-3-yl)-1,2,5-oxadiazol-3-yl)amino)ethyl)(methyl)(oxo)-16-sulfanylidene)cyclopropanesulfonamide 15b (0.65 g), yield 32.7%.

MS m/z (ESI): 551.0, 553.0 (M, M+2).

### Step 2: (Z)—N-(3-bromo-4-fluorophenyl)-4-((2-(N-(cyclopropylsulfonyl)-S-methylsulfonimidoyl)ethyl)amino)-N′-hydroxy-1,2,5-oxadiazole-3-carboximidamide 15

In a 100 mL one-necked flask, N-((2-((4-(4-(3-bromo-4-fluorophenyl)-5-oxo-4,5-dihydro-1,2,4-oxadiazol-3-yl)-1,2,5-oxadiazol-3-yl)amino)ethyl)(methyl)(oxo)-16-sulfanylidene)cyclopropanesulfonamide (0.65 g, 1.18 (mmol) was dissolved in tetrahydrofuran/methanol (10 mL/10 mL). Sodium hydroxide (236 mg, 5.95 mmol) dissolved in water (5 mL) was added to the above solution. The reaction mixture was stirred at room temperature for 2 hours and monitored by LC-MS. After the raw material was completely converted, the reaction was stopped. Saturated ammonium chloride solution (50 mL) was added, and the mixture was extracted with ethyl acetate (50 mL×2). The combined organic phases were washed with saturated sodium chloride (50 mL), dried over sodium sulfate and filtered. The filtrate was added with silica gel and dried, then directly purified by column chromatography with ethyl acetate/methanol (30/1 to 10/1) to obtain (Z)—N-(3-bromo-4-fluorophenyl)-4-((2-(N-(cyclopropylsulfonyl)-S-methylsulfonimidoyl)ethyl)amino)-N′-hydroxy-1,2,5-oxadiazole-3-carboximidamide 15 (350 mg), yield 54.0%.

MS m/z (ESI): 525.0, 527.0 (M, M+2).

¹HNMR (400 MHz, DMSO-d₆, ppm) δ 11.43 (s, 1H), 8.90 (s, 1H), 7.18 (t, J=8.8 Hz, 1H), 7.12 (dd, J₁=6.0 Hz, J₂=2.8 Hz, 1H), 6.77 (m, 1H), 6.55 (t, J=6.0 Hz, 1H), 3.93 (m, 1H), 3.80 (m, 3H), 3.47 (s, 3H), 2.64 (m, 1H), 0.95 (m, 4H).

### Example 16

### (Z)—N-(3-bromo-4-fluorophenyl)-4-((2-(N,S-dimethylsulfonimidoyl)ethyl)amino)-N′-hydroxy-1,2,5-oxadiazole-3-carboximidamide (16)

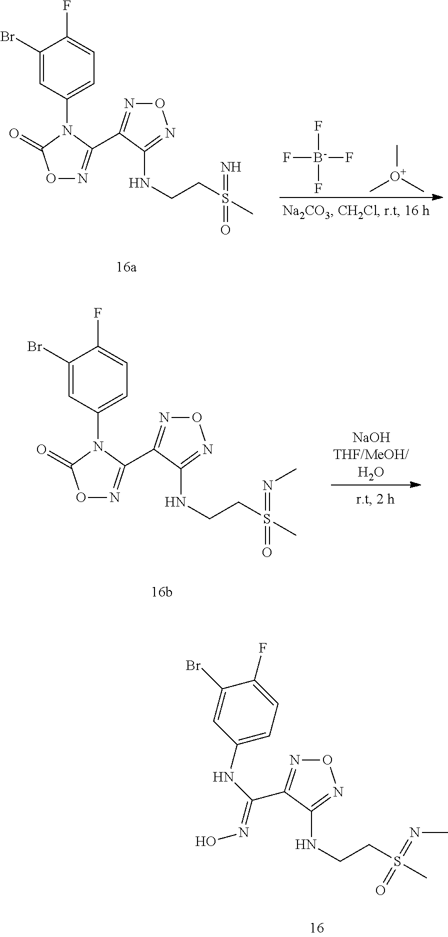

### Step 1: 4-(3-bromo-4-fluorophenyl)-3-(4-((2-(N,S-dimethylsulfonimidoyl)ethyl)amino)-1,2,5-oxadiazol-3-yl)-1,2,4-oxadiazol-5(4H)-one 16b

In a 100 mL one-necked flask, 4-(3-bromo-4-fluorophenyl)-3-(4-((2-(S-methylsulfonimidoyl)ethyl)amino)-1,2,5-oxadiazol-3-yl)-1,2,4-oxadiazol-5 (4H)-one (40 mg, 0.09 mmol), trimethyloxonium tetrafluoroborate (20 mg, 0.13 mmol) and dichloromethane (8 mL) were added. The reaction mixture was stirred for 15 minutes at room temperature. Then sodium carbonate (57.3 mg, 0.54 mmol) was added and the raction mixture was stirred overnight at room temperature. The reaction was stopped, and water (20 mL) was added. The mixture was extracted with ethyl acetate (20 mL×2). The combined organic phases were washed with saturated sodium chloride (30 mL), dried over anhydrous sodium sulfate and filtered. The filtrate was concentrated and purified by preparative silica gel plate (developing solvent: dichloromethane/methanol=10/1; eluent: ethyl acetate/methanol=10/1) 4-(3-bromo-4-fluorophenyl)-3-(4-((2-(N,S-dimethylsulfonimidoyl)ethyl)amino)-1,2,5-oxadiazol-3-yl)-1,2,4-oxadiazol-5(4H)-one 16b (20 mg, 50%).

MS m/z (ESI): 461.0, 463.0 (M, M+2).

¹H NMR (400 MHz, DMSO-d6, ppm) δ 8.11 (dd, J₁=6.4 Hz, J₂=2.8 Hz, 1H), 7.74 (m, 1H), 7.60 (t, J=8.8 Hz, 1H), 7.05 (t, J=6.0 Hz, 1H), 3.66 (dd, J, =12.4 Hz, J₂=6.4 Hz, 2H), 3.37 (t, J₂=6.4 Hz, 2H), 2.99 (s, 3H), 2.65 (s, 3H).

### Step 2: (Z)—N-(3-bromo-4-fluorophenyl)-4-((2-(N,S-dimethylsulfonimidoyl)ethyl)amino)-N′-hydroxy-1,2,5-oxadiazole-3-carboximidamide 16

In a 100 mL one-necked flask, 4-(3-bromo-4-fluorophenyl)-3-(4-((2-(N,S-4-(3-bromo-4-fluorophenyl)-3-(4-((2-(N,S-dimethylsulfonimidoyl)ethyl)amino)-1,2,5-xadiazol-3-yl)-1,2,4-oxadiazol-5(4H)-one (20 mg, 0.43 mmol) was dissolved in tetrahydrofuran/methanol (6 mL/6 mL), and then sodium hydroxide (9 mg, 0.22 mmol) dissolved in water (2 mL) was added to the above solution. The reaction mixture was stirred at room temperature for 2 hours and monitored by LC-MS. After the raw material was completely converted, the reaction was stopped. Saturated ammonium chloride solution (10 mL) was added, and the mixture was extracted with ethyl acetate (15 mL×2). The combined organic phases were washed with saturated sodium chloride (20 mL), dried over sodium sulfate and filtrated. The filtrate was concentrated and purified by preparative silica gel plate (developing solvent: dichloromethane/methanol=10/1; eluent: ethyl acetate/methanol=10/1) to obtain (Z)—N-(3-bromo-4-fluorophenyl)-(Z)—N-(3-bromo-4-fluorophenyl)-4-((2-(N,S-dimethylsulfonimidoyl)ethyl)amino)-N′-droxy-1,2,5-oxadiazole-3-carboximidamide 16 (13.0 mg, 68%).

MS m/z (ESI): 435.0, 437.0 (M, M+2).

¹H NMR (400 MHz, CDCl₃, ppm) δ 7.18 (dd, J₁=6.0 Hz, J₂=3.6 Hz, 1H), 7.18 (dd, J₁=5.6 Hz, J₂=2.8 Hz, 1H), 7.02 (t, J=8.4 Hz, 1H), 6.90 (m, 1H), 6.76 (t, J=6.0 Hz, 1H), 3.90 (m, 2H), 3.58 (m, 2H), 3.09 (s, 3H), 2.83 (s, 3H).

### Example 17

### (Z)—N-(3-bromo-4-fluorophenyl)-N′-hydroxy-4-((2-(N-methylethylsulfonimidoyl)ethyl)amino)-1,2,5-oxadiazole-3-carboximidamide (17)

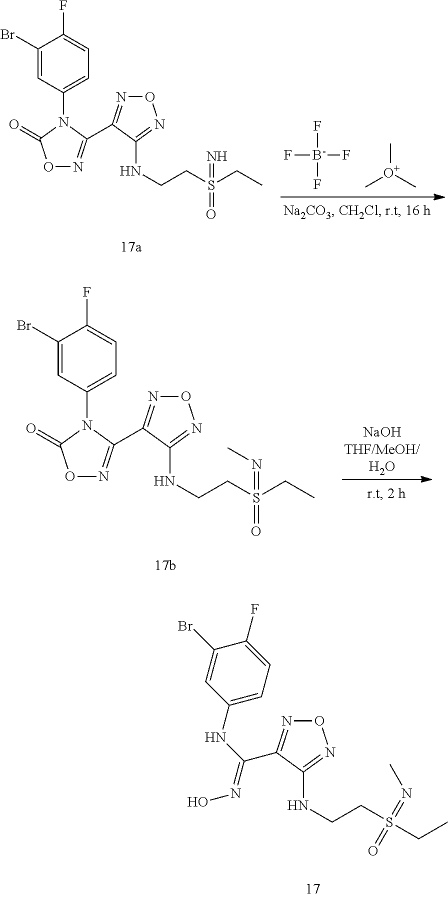

### Step 1: 4-(3-bromo-4-fluorophenyl)-3-(4-((2-(N-methylethylsulfonimidoyl)ethyl)amino)-1,2,5-oxadiazol-3-yl)-1,2,4-oxadiazol-5(4H)-one 17b

In a 100 mL one-necked flask, 4-(3-bromo-4-fluorophenyl)-3-(4-((2-(S-ethylsulfonimidoyl)ethyl)amino)-1,2,5-oxadiazol-3-yl)-1,2,4-oxadiazol-5 (4H)-one (300 mg, 0.65 mmol), trimethyloxonium tetrafluoroborate (115 mg, 0.78 mmol) and dichloromethane (30 mL) were added. The reaction mixture was stirred at room temperature for 15 minutes, and then sodium carbonate (414 mg, 3.9 mmol) was added. The reaction mixture was stirred overnight at room temperature. After the reaction was stopped, water (50 mL) was added, and the mixture was extracted with ethyl acetate (50 mL×2). The combined organic phases were washed with saturated sodium chloride (50 mL), dried over anhydrous sodium sulfate and filtered. The filtrate was concentrated and purified by preparative silica gel plate (developing solvent: methylene chloride/methanol=15/1; eluent: ethyl acetate/methanol=15/1) to obtain 4-(3-bromo-4-fluorophenyl)-3-(4-((2-(N-methylethylsulfonimidoyl)ethyl)amino)-1,2,5-oxadiazol-3-yl)-1,2,4-oxadiazol-5(4H)-one 17b (130 mg, 42.1%).

MS m/z (ESI): 475.0, 477.0 (M, M+2).

¹H NMR (400 MHz, DMSO-d₆, ppm) δ 8.12 (dd, J1=6.0 Hz, J2=2.8 Hz, 1H), 7.74 (m, 1H), 7.60 (t, J=8.8 Hz, 1H), 7.00 (t, J=6.0 Hz, 1H), 3.63 (dd, J₁=12.8 Hz, J₂=6.4 Hz, 2H), 3.40 (m, 1H), 3.30 (m, 1H), 3.16 (m, 2H), 2.64 (s, 3H), 1.22 (t, J=7.2 Hz, 3H).

### Step 2: (Z)—N-(3-bromo-4-fluorophenyl)-N′-hydroxy-4-((2-(N-methylethylsulfonimidoyl)ethyl)amino)-1,2,5-oxadiazole-3-carboximidamide 17

In a 100 mL one-necked flask, 4-(3-bromo-4-fluorophenyl)-3-(4-((2-(N-methylethylsulfonimidoyl)ethyl)amino)-1,2,5-oxadiazol-3-yl)-1,2,4-oxadiazol-5(4H)-one (130 mg, 0.27 mmol) was dissolved in tetrahydrofuran/methanol (8 mL/8 mL). Sodium hydroxide (55 mg, 1.36 mmol) dissolved in water (5 mL) was added to the above solution. The mixture was stirred at room temperature for 2 hours and monitored by LC-MS. After the raw material was completely converted, the reaction was stopped. Saturated ammonium chloride solution (30 mL) was added, and the mixture was extracted with ethyl acetate (30 mL×2). The combined organic phases were washed with saturated sodium chloride (30 mL), dried over sodium sulfate and filtrated. The filtrate was concentrated and purified by preparative silica gel plate (developing solvent:dichloromethane/methanol=10/1; eluent: ethyl acetate/methanol=10/1) to obtain (Z)—N-(3-bromo-4-fluorophenyl)-4-((2-(N,S-dimethylsulfonimidoyl)ethyl)amino)-N′-hydroxy-1,2,5-oxidazole-3-carboximidamide 17 (76.6 mg, 63.2%).

MS m/z (ESI): 448.0, 450.0 (M, M+2).

¹HNMR (400 MHz, DMSO-d₆, ppm) δ 11.45 (s, 1H), 8.89 (s, 1H), 7.18 (t, J=8.8 Hz, 1H), 7.10 (dd, J₁=6.0 Hz, J₂=2.8 Hz, 1H), 6.76 (m, 1H), 6.56 (t, J=6.0 Hz, 1H), 3.58 (dd, J₁=12.8 Hz, J₂=6.4 Hz, 2H), 3.31 (m, 2H), 2.63 (s, 3H), 1.22 (t, J=7.2 Hz, 3H).

### Example 18

### (R,Z)—N-(3-bromo-4-fluorophenyl)-4-((2-(N-(cyclopropylsulfonyl)-S-methylsulfonimidoyl)ethyl)amino)-N′-hydroxy-1,2,5-oxadiazole-3-carboximidamide (18-(R,Z)) and

### (S,Z)—N-(3-bromo-4-fluorophenyl)-4-((2-(N-(cyclopropylsulfonyl)-S-methylsulfonimidoyl)ethyl)amino)-N′-hydroxy-1,2,5-oxadiazole-3-carboximidamide (18-(S,Z)

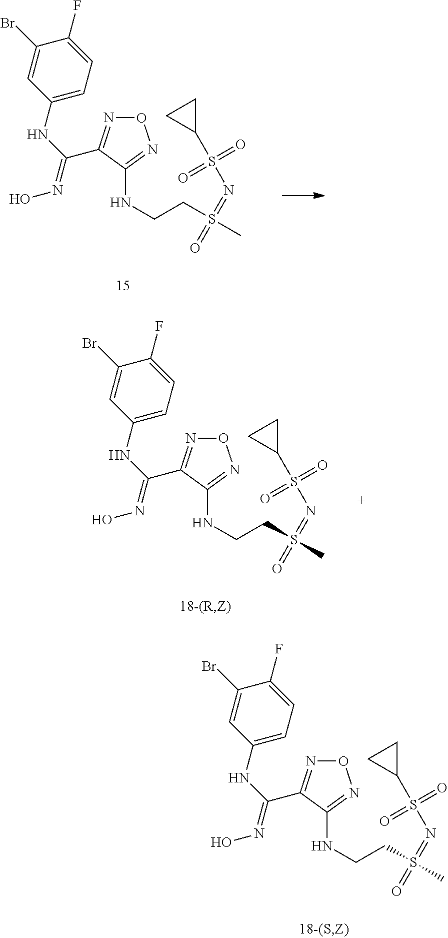 [SMILES: CS(=O)(CCNc1nonc1/C(=N/O)Nc1ccc(F)c(Br)c1)=NS(=O)(=O)C1CC1 ; C[S@@](=O)(CCNc1nonc1/C(=N/O)Nc1ccc(F)c(Br)c1)=NS(=O)(=O)C1CC1 ; C[S@](=O)(CCNc1nonc1/C(=N/O)Nc1ccc(F)c(Br)c1)=NS(=O)(=O)C1CC1]

### The Preparation Method is as Follows:

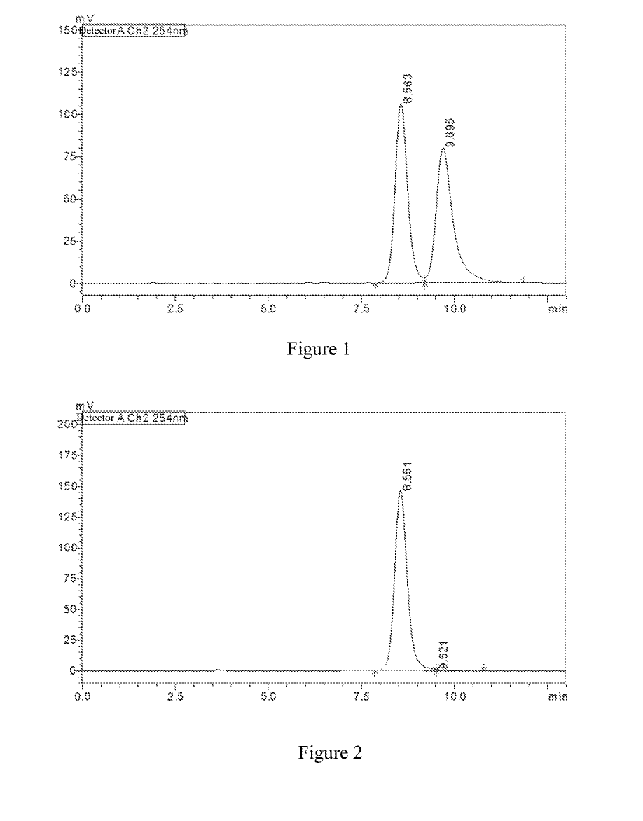

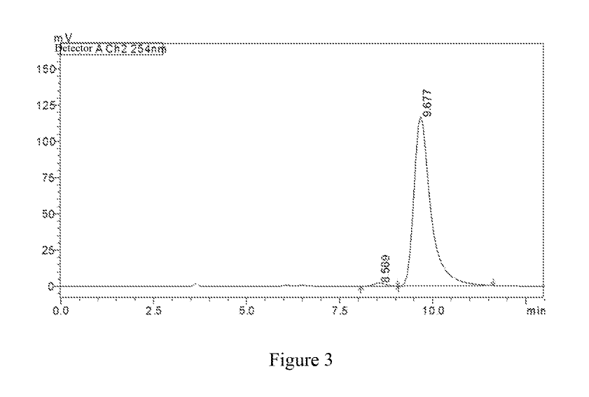

In the present invention, 2.3457 g of the compound of Example 15 (see detection spectrum, FIG. 1) was subjected to chiral isomer separation using a preparation apparatus (Japan, YMC, K-Prep LAB100S type supercritical fluid chromatography preparation apparatus) and a Daicel chiral column (AD-H 4.6*250, filler particle size: 5 m). The sample solutions at 8.56 min and 9.69 min were respectively collected. The solvent was removed by rotary evaporation to obtain optical isomer \textcircled{1} at 8.56 min, 0.9744g (ee % value: 99.322%, the detection spectrum is shown in FIG. 2) and optical isomer \textcircled{2} at 9.69 min, 0.9552g (ee % value: 98.676%, the detection spectrum is in FIG. 3).

Eluent for preparation: (A: ethanol, B: 0.1% DEA n-hexane, A: B=30:70, volume ratio); detection wavelength: 214 nm; column temperature: 20° C.

The chiral purity analysis method was as follows:

| | |
|---|---|
| Chromatographic column type | chiral column OJ-H |
| Column size | 0.46 cm I.D. × 25 cm L |
| Injection volume | 1.0 μL |
| Mobile phase | MeOH = 100% |
| Flow rate | 1.0 mL/min |
| Detection wavelength | UV 254 nm |
| Column temperature | 35° C. |

### The Optical Rotation was Determined as Follows:

Optical rotation tester: Perkin Elmer (PE), model: Perkin Elmer 341. The determination results were as follows:

### Blank:

| WL (nm) | Arc [°] | Energy [#] | Temp. [° C.] |
|---|---|---|---|
| 589 | 0 | 75 | 20 |

### Sample:

| | Concentration | | | | |
|---|---|---|---|---|---|
| Sample | [g/mL] | Solvent | WL (nm) | Arc [°] | OROT [°] |
| Optical isomer 18-(R,Z) [symbol] | 0.01007 (C = 1) | MeCN | 589 | −0.110 | −10.9 |
| Optical isomer 18-(S,Z) [symbol] | 0.00999 (C = 1) | MeCN | 589 | +0.096 | +9.6 |

### Biological Evaluation

### I. Enzymatic Test for IDO Activity Inhibition

Human indoleamine 2,3-dioxygenase (IDO) was purchased from BPS Bioscience Inc. The enzymatic reaction of idoleamine 2,3-dioxygenase (IDO) was performed in a 96-well plate with a reaction volume of 20 μL. The reaction conditions were: 40 nM IDO enzyme, 0.2 mM L-tryptophan, 50 mM KPB (pH 6.5) buffer, 20 mM sodium L-L-ascorbate, 10 μM methylene blue, 0.2 mg/mL catalase, different concentrations of compounds containing <1% dimethyl sulfoxide. After reacting for 60 minutes at 30° C., 5 μL of 30% (w/v) trichloroacetic acid (in 50 mM KPB buffer) weres added to each well. The plate was incubated for 30 minutes at 50° C. to hydrolyze N-formyl-kynurenine to kynurenine. 25 μL of 2% (w/v) p-(dimethylamino)benzaldehyde (p-DMBA)/glacial acetic acid solution were added to each well. The absorbance at 490 nm was read on a BioTek Synergy H1 microplate reader (Molecular Devices).

The stock solution of the test compound was prepared to 10 mM with dimethyl sulfoxide, diluted with dimethyl sulfoxide to the highest concentration of the test during the experiment, then diluted in 1:3 gradient, and generally diluted to 8 to 10 concentration points. Duplicate wells were set for each concentration point, and one reference compound was included in each experiment. The original data of the absorbance at 490 nm read on a microplate reader were analyzed. The inhibition of IDO enzyme activity was calculated at different concentrations of the test compound. The half-inhibitory concentration IC₅₀ value of the compound was obtained by non-linear fitting analysis of inhibition percentage data by GraphPad Prism software.

### II. Cell Model Test for IDO Activity Inhibition

Interferon γ induced the expression of IDO in HeLa cells. This model was used to test the inhibitory activity of compounds on indoleamine 2,3-dioxygenase (IDO). The culture medium of HeLa cells (ATCC) was phenol red-free RPMI-1640 containing 100 μM L-tryptophan. The stock solution of the test compound was prepared to 10 mM with dimethyl sulfoxide, and diluted with dimethyl sulfoxide to the highest concentration of the test during the experiment, then diluted in three-fold gradient, and generally diluted to 8 to 10 concentration points. Duplicate wells were set for each concentration point. The final concentration of DMSO was 0.5%, and internal reference compound was included in each experiment.

The procedure of the experiment was as follows: 20,000 HeLa cells (ATCC) per well were added on a 96-well culture plate and incubated overnight. After 24 hours, interferon γ (final concentration of 50 ng/mL) and different concentrations of the test compound and the internal reference compound were added to the incubated cells. After 24 hours, 140 μL of the supernatant/well was transferred to a new 96-well plate, and 10 μL of 6.1 N trichloroacetic acid was added to each well. The plate was incubated for 30 minutes at 50° C. to hydrolyze N-formyl-kynurenine to kynurenine. The reaction mixture was centrifuged at 2500 rpm for 10 minutes to remove the precipitate, and the supernatant (100 μL) was transferred to another new 96-well plate. 100 μL of 2% (W/V) p-(dimethylamino)benzaldehyde (p-DMBA)/glacial acetic acid solution was added to each well. The absorbance at 490 nm was read on a BioTek Synergy H1 microplate reader (Molecular Devices).

The original data of the absorbance at 490 nm read on a microplate reader were analyzed. The inhibition of IDO enzyme activity was calculated at different concentrations of the test compound. The half-inhibitory concentration IC₅₀ value of the compound was obtained by non-linear fitting analysis of inhibition percentage data by GraphPad Prism software.

The example compounds of the present invention were respectively determined by the above two test methods. The IC₅₀ value results of the enzymatic and cytochemical IDO inhibitory activity s are shown in the following table:

| | | IDO Inhibitory Activity | |
|---|---|---|---|
| | | Enzymatic Test | Cytological Test |
| Example No. | | IC₅₀ (nM) | IC₅₀ (nM) |
| Example 1 | | 70 | 15 |
| Example 2 | | 71 | 18 |
| Example 3 | | 56 | 20 |
| Example 5 | | 73 | 93 |
| Example 5 | | 64 | 44 |
| Example 7 | | 53 | 53 |
| Example 8 | | 76 | 33 |
| Example 10 | | 19 | 37 |
| Example 11 | | 42 | 38 |
| Example 13 | | 64 | 47 |
| Example 14 | | 38 | 12 |
| Example 15 | | 27 | 10 |
| Example 16 | | 66 | 19 |
| Example 17 | | 75 | 16 |
| Example 18 | Optical Isomer [symbol] | 19 | 11 |
| | Optical Isomer [symbol] | 24 | 9 |

The test results demonstrated that the example compounds of the present invention had good enzymatic and cytological IDO inhibitory activities.

### III. Pharmacokinetic (PK) Analysis of Rat Plasma

The pharmacokinetics test of the test compound was performed with Sprague Dawley (SD) rats (Shanghai Slac Laboratory Animal Co., LTD).

Mode of administration: Single gavage.

U Dosage: 20 mg/10 mL/kg.

Formulation prescription: 3% dimethylacetamide and 20% hydroxypropyl-3-cyclodextrin.

Sampling points: before administration and 15 minutes, 0.5, 1, 2, 4, 6, 8 and 24 hours after administration.

Plasma sampling and sample processing:
1) 0.2 ml of intravenous blood was collected and placed in an EDTA-2K tube. The blood was centrifuged at 4° C. for 5 minutes at 6,000 rpm to separate the plasma, which was stored at −80° C.
2) 160 μL of acetonitrile were added to 40 μL of plasma sample, standard, and internal reference. The mixture was shaken vertically for 3 minutes, and centrifuged at 4000 rpm for 10 minutes. 100 μL of the supernatant were taken, and then added with 100 μL of deionized water and mixed well. 100 μL of the resulting solution was taken for LC/MS/MS analysis. The instrument for plasma LC/MS/MS analysis was AB Sciex API 4000.

Liquid Chromatography Analysis:
Liquid chromatography conditions: Shimadzu LC-20AD pump
Chromatographic column: phenomenex Gemiu 5 μm C18 50×4.6 mm
Mobile phase: solution A was 0.1% formic acid solution, and solution B was acetonitrile
Flow rate: 0.8 mL/min
Elution time: 0-3.01 minutes and the eluent was as follows:

| Time/minute | Solution A | Solution B |
|---|---|---|
| 0.01 | 70% | 30% |
| 1 | 10% | 90% |
| 2 | 10% | 90% |
| 2.01 | 70% | 30% |
| 3 | 70% | 30% |

Mass spectrometry analysis: setup conditions of mass spectrometer: positive ion electrospray ionization (ESI) mode.
Experimental results: the main parameters of pharmacokinetics were calculated by WinNonlin 6.1 and the experimental results are shown in the following table:

| | | | | Example | Example | Reference |
|---|---|---|---|---|---|---|
| | | Example | Example | 18- | 18- | compound |
| Parameters | Example 1 | 14 | 15 | [symbol] | [symbol] | (INCB-24360) |
| Tₘₐₓ (h) | 0.5 | 0.5 | 0.5 | 0.5 | 0.5 | 1 |
| Cₘₐₓ (ng/mL) | 3675 | 1048 | 1354 | 2774 | 2016 | 306 |
| AUC_{0-∞}g/mL * h) | 8617 | 7544 | 5178 | 7316 | 4611 | 2447 |
| (ng/mL * h) | | | | | | |
| T_{1/2} (h) | 2.7 | 5.5 | 3.54 | 2.03 | 1.4 | 3.5 |

The experimental results showed that the example compounds of the present invention were obviously better than the reference compound (INCB-24360), and had better pharmacokinetics. The main pharmacokinetic parameters: maximum drug concentration (Cmax) and drug exposure (AUC) were greatly improved compared with the reference compound (INCB-24360).

### IV. Anti-Tumor Effect of the Example Compounds in PAN02 Tumor-Bearing Mouse Model

The invention used the PAN02 tumor-bearing mouse model to test the anti-tumor effect of the example compounds. The PAN02 tumor-bearing mouse model was the mouse pancreatic cancer cell line PAN02 purchased from Guangzhou Ginnio Biological Technology Co., Ltd., and the culture medium used was DMEM containing 10% fetal bovine serum. The mouse strain used for tumor-bearing was C57/BL6 purchased from Shanghai Slac Laboratory Animal Co., Ltd. At the time of implanting, the PAN02 cells in the logarithmic growth phase were collected, and mixed with the BDMatrigel matrix gel that reduced the growth factor to 50 million cells/ml. Each mouse was implanted subcutaneously with 100 μL of 5 million cells. When the tumor grew to about 100 cubic millimeters, animals were randomly divided into groups with 8 animals per group. Drug administration was started (D0).

Mode of administration: intragastrical administration, twice a day.

Dosage: 50 mg/10 mL/kg.

Formulation prescription: 3% two methyl acetamide and 20% hydroxypropyl-β-cyclodextrin.
Administration period and tumor measurement: The administration period was 13 days. The tumor volume was measured, the mice were weighed, and the data were recorded, 3 times a week. The calculation formula of tumor volume (V) is: V=½a×b2, where a and b represent length and width respectively. The calculation formula of T/C is: T/C (%)=100×ΔT/ΔC. Tumor inhibition rate (%)=1−T/C (%).
Experimental results: the anti-tumor effect of the compound of Example 15 in PAN02 tumor-bearing mouse model is shown in the following table.

| | | | | | Tumor | |
|---|---|---|---|---|---|---|
| | | | | | inhibition | |
| | | Average tumor | | T/C | rate | |
| | Administration | volume (mm³ ± SEM) | | (%) | (%) | P-value |
| Group | date | D0 | D12 | D12 | D12 | D12 |
| Vehicle | D0-D12 | 105.4 ± 3.9 | 336.5 ± 25.4 | — | — | — |
| INCB | D0-D12 | 102.8 ± 3.4 | 242.1 ± 23.4 | 60.3% | 39.7% | <0.05 |
| 24360 | | | | | | |
| 50 mg/kg | | | | | | |
| Example 15 | D0-D12 | 105.0 ± 3.4 | 163.3 ± 11.0 | 25.2% | 74.8% | <0.01 |
| 50 mg/kg | | | | | | |

As can be seen from the table, the tumor inhibition rate of the compound of Example 15 in the PAN02 tumor-bearing mice was 74.8% at a dose of 50 mg/kg, which was significantly higher than that of the reference positive compound INCB 24360 (tumor inhibition rate of 39.7%).

### V. Anti-Tumor Effect of the Example Compounds in Colon26 Tumor-Bearing Mouse Model

The present invention further used the Colon26 tumor-bearing mouse model to test the anti-tumor effect of the example compounds. The Colon26 tumor-bearing mouse model was the mouse rectal cancer cell line Colon26 purchased from Guangzhou Ginio Biological Technology Co., Ltd., and the culture medium used was RPMI1640 containing 10% fetal bovine serum. The mouse strain used for tumor-bearing was Balb/c purchased from Sino-British SIPPR/BK Lab Animal Co., Ltd. At the time of implanting, the Colon26 cells in logarithmic growth phase were collected and mixed to 10 million cells/ml. Each mouse was implanted subcutaneously with 100 μl of one million cells. When the tumor grew to about 100 cubic millimeters, animals were randomly divided into groups with 8 animals per group. Drug administration was started (D0).

Mode of administration: intragastric administration, twice a day.

Dosage: 50 mg/10 mL/kg.

Formulation prescription: 3% dimethylacetamide and 20% hydroxypropyl-β-cyclodextrin.
Administration period and tumor measurement: The administration period was 13 days. The tumor volume was measured, the mice were weighed, and the data were recorded, 3 times per week. The calculation formula of tumor volume (V) is: V=½a×b2 where a and b represent length and width, respectively. The calculation formula of T/C is: T/C (%)=100×ΔT/ΔC. Tumor inhibition rate (%)=1−T/C (%).
Experimental results: The antitumor effect of the compound of Example 15 in Colon26 tumor-bearing mouse model is shown in the following table.

| | | | | | Tumor | |
|---|---|---|---|---|---|---|
| | | | | | inhibition | |
| | | Average tumor volume | | T/C | rate | |
| | Administration | (mm³ ± SEM) | | (%) | (%) | P-value |
| Group | date | D0 | D12 | D12 | D12 | D12 |
| Vehicle | D0-D12 | 95.9 ± 2.8 | 2269.2 ± 172.5 | — | — | — |
| INCB | D0-D12 | 95.3 ± 2.4 | 565.2 ± 10.9 | 21.6% | 78.4% | <0.01 |
| 24360 | | | | | | |
| 25 mg/kg | | | | | | |
| Example | D0-D12 | 95.5 ± 2.0 | 212.7 ± 28.9 | 5.4% | 94.6% | <0.01 |
| 15 | | | | | | |
| 25 mg/kg | | | | | | |

As can be seen from the table, the tumor inhibition rate of the compound of Example 15 in the Colon26 tumor-bearing mice was 94.6% at the dose of 25 mg/kg, which was significantly higher than that of the reference positive compound INCB 24360 (tumor inhibition rate of 78.4%).

Under the same experimental conditions, the dosage was adjusted. The anti-tumor effect of Example 15 and its optical isomers 18-\textcircled{1} and 18-\textcircled{2} in Colon26 tumor-bearing mouse model is shown in the following table:

| Examples and administration | |
|---|---|
| dosages thereof | Tumor inhibition rate (%) D12 |
| Example 15 10 mg/kg | 71.10% |
| Example 18- [symbol] 10 mg/kg | 72.54% |
| Example 18- [symbol] 10 mg/kg | 68.49% |

From the results in the table, it can be seen that the optically pure compound 18 obtained by resolving the compound of Example 15 had a comparable tumor inhibition rate in Colon26 tumor-bearing mice, and had a good inhibitory effect.

1. A compound of formula (I), a stereoisomer or a pharmaceutically acceptable salt thereof, 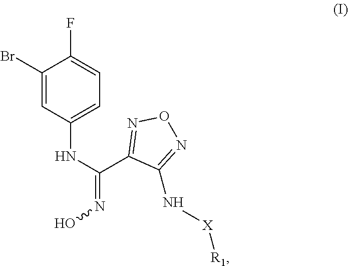
wherein: [symbol]
is a Z configuration or E configuration;
X is selected from the group consisting of C₁₋₈ alkyl and C₃₋₈ cycloalkyl, optionally substituted by one or more groups selected from the group consisting of deuterium, halogen, hydroxy, thiol, cyano, nitro, azido, C₁₋₈ alkyl, C₂₋₈ alkenyl, C₂₋₈ alkynyl, haloC₁₋₈ alkyl, C₃₋₈ cycloalkyl, 3-8 membered heterocyclyl, 3-8 membered heterocyclyloxy, 3-8 membered heterocyclylthio, C₅₋₁₀ aryl, C₅₋₁₀ aryloxy, C₅₋₁₀ arylthio, 5-10 membered heteroaryl, 5-10 membered heteroaryloxy, 5-10 membered heteroarylthio, —C₀₋₈—S(O)ᵣR₄, —C₀₋₈—O—R₅, —C₀₋₈—C(O)OR₅, —C₀₋₈—C(O)R₆, —C₀₋₈—O—C(O)R₆, —C₀₋₈—NR₇R₈, —C₀₋₈—C(O)NR₇R₈, —N(R₇)—C(O)R₆ and —N(R₇)—C(O)OR₅;
R₁ is selected from the group consisting of: 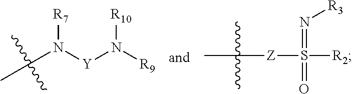
Y is selected from the group consisting of —S(O)₂— and —C(O)—C(O)—;
Z is selected from the group consisting of a bond, O, S and —NR₇—;
R₂ is selected from the group consisting of hydrogen, deuterium, C₁₋₈ alkyl, C₂₋₈ alkenyl, C₂₋₈ alkynyl, C₃₋₈ cycloalkyl, 3-8 membered heterocyclyl, C₅₋₁₀ aryl, 5-10 membered heteroaryl and C₀₋₈ alkylcarbonyl,
optionally substituted by one or more groups selected from the group consisting of halogen, hydroxy, thiol, cyano, nitro, azido, C₁₋₈ alkyl, C₂₋₈ alkenyl, C₂₋₈ alkynyl, haloC₁₋₈ alkyl, C₃₋₈ cycloalkyl, 3-8 membered heterocyclyl, 3-8 membered heterocyclyloxy, 3-8 membered heterocyclylthio, C₅₋₁₀ aryl, C₅₋₁₀ aryloxy, C₅₋₁₀ arylthio, 5-10 membered heteroaryl, 5-10 membered heteroaryloxy, 5-10 membered heteroarylthio, —C₀₋₈—S(O)ᵣR₄, —C₀₋₈—O—R₅, —C₀₋₈—C(O)OR₅, —C₀₋₈—C(O)R₆, —C₀₋₈—O—C(O)R₆, —C₀₋₈—NR₇R₈, —C₀₋₈—C(O)NR₇R₈, —N(R₇)—C(O)R₆ and —N(R₇)—C(O)OR₅;
R₃ is selected from the group consisting of hydrogen, deuterium, hydroxy, amino, C₁₋₈ alkyl, C₂₋₈ alkenyl, C₃₋₈ cycloalkyl, 3-8 membered heterocyclyl, C₅₋₁₀ aryl, 5-10 membered heteroaryl, C₁₋₈ alkoxy, C₃₋₈ cycloalkoxy, 3-8 membered heterocyclyloxy, C₅₋₁₀ aryloxy, 5-10 membered heteroaryloxy, —C₀₋₈—S(O)ᵣR₄, —C₀₋₈—C(O)OR₅, —C₀₋₈—O—C(O)R₆, —C₀₋₈—NR₇R₈, —C₀₋₈—C(O)NR₇R₈, —N(R₇)—C(O)R₆ and —N(R₇)—C(O)OR₅,
optionally substituted by one or more groups selected from the group consisting of halogen, hydroxy, thiol, cyano, nitro, azido, C₁₋₈ alkyl, C₂₋₈ alkenyl, C₂₋₈ alkynyl, haloC₁₋₈ alkyl, C₃₋₈ cycloalkyl, 3-8 membered heterocyclyl, 3-8 membered heterocyclyloxy, 3-8 membered heterocyclylthio, C₅₋₁₀ aryl, C₅₋₁₀ aryloxy, C₅₋₁₀ arylthio, 5-10 membered heteroaryl, 5-10 membered heteroaryloxy, 5-10 membered heteroarylthio, —C₀₋₈—S(O)ᵣR₄, —C₀₋₈—O—R₅, —C₀₋₈—C(O)OR₅, —C₀₋₈—C(O)R₆, —C₀₋₈—O—C(O)R₆, —C₀₋₈—NR₇R₈, —C₀₋₈—C(O)NR₇R₈, —N(R₇)—C(O)R₆ and —N(R₇)—C(O)OR₅;
R₄ is selected from the group consisting of hydrogen, deuterium, C₁₋₈ alkyl, C₂₋₈ alkenyl, C₃₋₈ cycloalkyl, haloC₁₋₈ alkyl, phenyl, p-methylphenyl, amino, mono C₁₋₈ alkylamino, di C₁₋₈ alkylamino and C₁₋₈ alkanoylamino;
R₅ is selected from the group consisting of hydrogen, deuterium, C₁₋₈ alkyl, C₃₋₈ cycloalkyl, haloC₁₋₈ alkyl, and hydroxyC₁₋₈ alkyl;
R₆ is selected from the group consisting of hydrogen, deuterium, C₁₋₈ alkyl, C₁₋₈ alkoxy, C₃₋₈ cycloalkyl, C₃₋₈ cycloalkoxy, haloC₁₋₈ alkyl, haloC₁₋₈ alkoxy, hydroxyC₁₋₈ alkyl and hydroxyC₁₋₈ alkoxy;
R₇, R₈, R₉, and R₁₀ are each independently selected from the group consisting of hydrogen, deuterium, hydroxy, C₁₋₈ alkyl, hydroxyC₁₋₈ alkyl, C₁₋₈ alkoxy, C₂₋₈ alkenyl, C₂₋₈ alkynyl, C₃₋₈ cycloalkyl, 3-8 membered heterocyclyl, C₅₋₁₀ aryl, 5-10 membered heteroaryl and C₁₋₈ alkanoyl, or R₇ and R₈, R₉ and R₁₀ together with the nitrogen atom to which they are attached form a 3-8 membered heterocycloalkyl,
optionally substituted by one or more groups selected from the group consisting of halogen, hydroxy, thiol, cyano, nitro, acetamido, azido, sulfonyl, methylsulfonyl, C₁₋₈ alkyl, trifluoromethyl, C₂₋₈ alkenyl, C₂₋₈ alkynyl, C₃₋₈ cycloalkyl, 3-8 membered heterocyclyl, C₁₋₈ alkoxy, C₁₋₈ alkoxycarbonyl, C₁₋₈ alkylcarbonyl, C₁₋₈ alkylcarbonyloxy, 3-8 membered heterocyclyloxy, 3-8 membered heterocyclylthio, C₅₋₁₀ aryl, C₅₋₁₀ aryloxy, C₅₋₁₀ arylthio, 5-10 membered heteroaryl, 5-10 membered heteroaryloxy, 5-10 membered heteroarylthio, amino, mono C₁₋₈ alkylamino, and di C₁₋₈ alkylamino; and
r is 0, 1, or 2.

2. The compound of formula (I), the stereoisomer or the pharmaceutically acceptable salt thereof according to claim 1, which is a compound of formula (II): 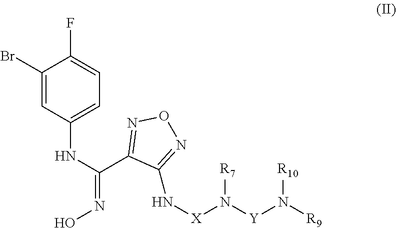
wherein:
X is selected from the group consisting of C₁₋₆ alkyl and C₃₋₈ cycloalkyl, optionally substituted by one or more groups selected from the group consisting of deuterium, halogen, hydroxy, thiol, cyano, nitro, azido, C₁₋₈ alkyl, haloC₁₋₈ alkyl and C₃₋₈ cycloalkyl; and
R₇, R₉, and R₁₀ are each independently selected from the group consisting of hydrogen, deuterium, hydroxy, C₁₋₈ alkyl, hydroxyC₁₋₈ alkyl, C₁₋₈ alkoxy, C₂₋₈ alkenyl, C₂₋₈ alkynyl, C₃₋₈ cycloalkyl, 3-8 membered heterocyclyl, C₅₋₁₀ aryl, C₅₋₁₀ aryl substituted by C₁₋₈ alkyl, 5-10 membered heteroaryl, C₁₋₈ alkanoyl and —C₀₋₈—C(O)OR₅, or R₉ and R₁₀ together with the nitrogen atom to which they are attached form a 5-6 membered heterocycloalkyl,
optionally substituted by one or more groups selected from the group consisting of halogen, hydroxy, thiol, cyano, nitro, acetamido, azido, sulfonyl, methylsulfonyl, C₁₋₈ alkyl, trifluoromethyl, C₃₋₈ cycloalkyl, 3-8 membered heterocyclyl, C₁₋₈ alkoxy, C₁₋₈ alkoxycarbonyl, C₁₋₈ alkylcarbonyl, C₁₋₈ alkylcarbonyloxy, 3-8 membered heterocyclyloxy, 3-8 membered heterocyclylthio, C₅₋₁₀ aryl, C₅₋₁₀ aryloxy, C₅₋₁₀ arylthio, 5-10 membered heteroaryl, 5-10 membered heteroaryloxy, 5-10 membered heteroarylthio, amino, mono C₁₋₈ alkylamino, and di C₁₋₈ alkylamino.

3. The compound of formula (I), the stereoisomer or the pharmaceutically acceptable salt thereof according to claim 1, which is selected from the group consisting of a compound of formula (IIA) and a compound of formula (IIB): 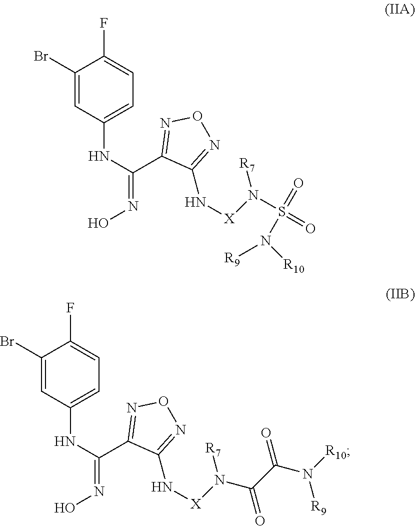
wherein:
X is selected from the group consisting of ethyl, cyclobutyl and cyclohexyl, optionally substituted by one or more groups selected from the group consisting of deuterium, halogen, hydroxy, thiol, cyano, nitro, trifluoromethyl, C₁₋₈ alkyl, and C₃₋₈ cycloalkyl; and
R₇, R₉, and R₁₀ are each independently selected from the group consisting of hydrogen, deuterium, hydroxy, C₁₋₈ alkyl, hydroxyC₁₋₈ alkyl, C₁₋₈ alkoxy, C₃₋₈ cycloalkyl, 3-8 membered heterocyclyl, C₅₋₁₀ aryl, C₅₋₁₀ aryl substituted by C₁₋₈ alkyl, 5-10 membered heteroaryl, C₁₋₈ alkanoyl and —C₀₋₈—C(O)OR₅, or R₉ and R₁₀ together with the nitrogen atom to which they are attached form a 5-6 membered heterocycloalkyl.

4. A compound selected from the group consisting of: 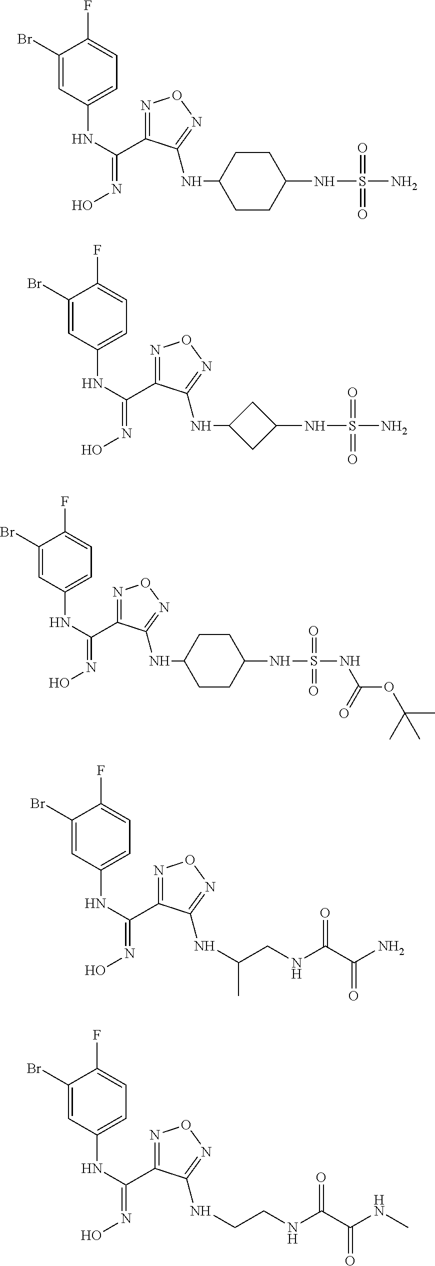 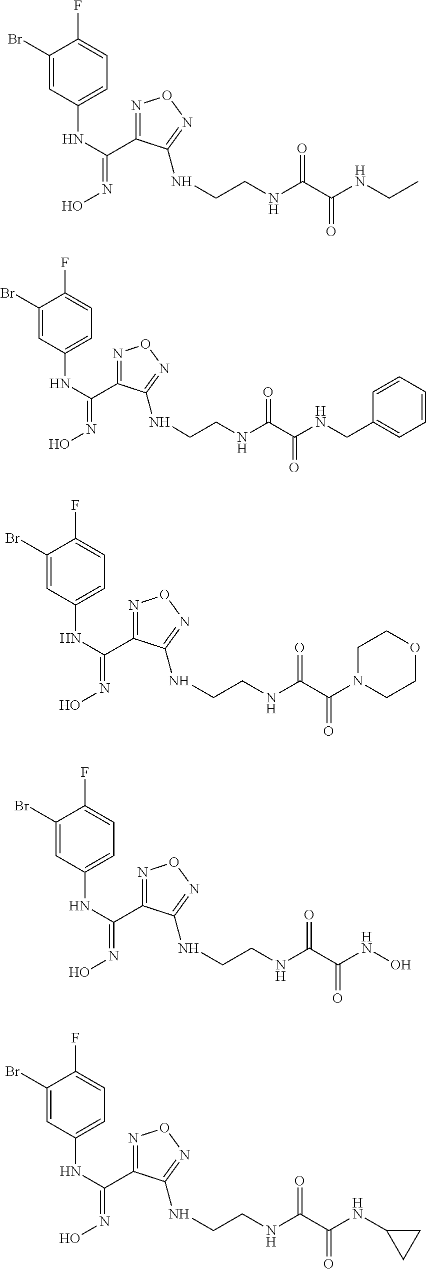 [SMILES: CCNC(=O)C(=O)NCCNc1nonc1/C(=N/O)Nc1ccc(F)c(Br)c1 ; O=C(NCCNc1nonc1/C(=N/O)Nc1ccc(F)c(Br)c1)C(=O)NCc1ccccc1 ; O=C(NCCNc1nonc1/C(=N/O)Nc1ccc(F)c(Br)c1)C(=O)N1CCOCC1 ; O=C(NO)C(=O)NCCNc1nonc1/C(=N/O)Nc1ccc(F)c(Br)c1 ; O=C(NCCNc1nonc1/C(=N/O)Nc1ccc(F)c(Br)c1)C(=O)NC1CC1] 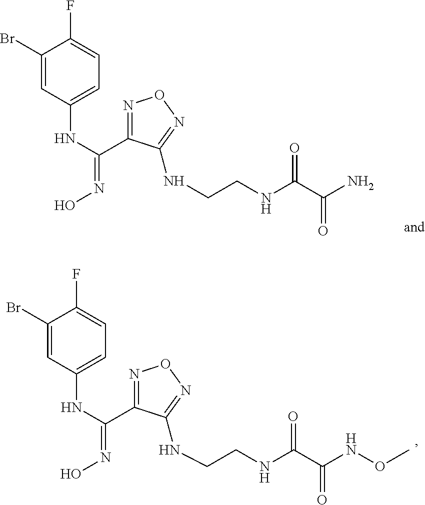 [SMILES: NC(=O)C(=O)NCCNc1nonc1/C(=N/O)Nc1ccc(F)c(Br)c1 ; CONC(=O)C(=O)NCCNc1nonc1/C(=N/O)Nc1ccc(F)c(Br)c1]
or a stereoisomer, or a pharmaceutically acceptable salt thereof.

5. The compound of formula (I), the stereoisomer or the pharmaceutically acceptable salt thereof according to claim 1, which is a compound of formula (III): 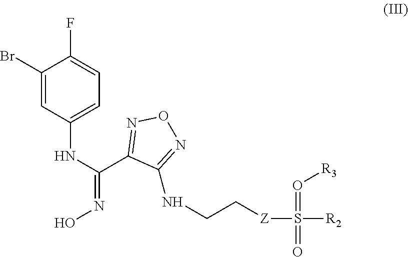
Z is selected from the group consisting of a bond and —NR₇—;
R₂ is selected from the group consisting of hydrogen, deuterium, and C₁₋₈ alkyl;
R₃ is selected from the group consisting of deuterium, hydroxy, amino, C₁₋₈ alkyl, C₃₋₈ cycloalkyl, 3-8 membered heterocyclyl, C₅₋₁₀ aryl, 5-10 membered heteroaryl, C₁₋₈ alkoxy, C₃₋₈ cycloalkoxy, 3-8 membered heterocyclyloxy, C₅₋₁₀ aryloxy, 5-10 membered heteroaryloxy, —C₀₋₈—S(O)ᵣR₄, —C₀₋₈—C(O)OR₅ and —C₀₋₈—OC(O)R₆;
R₄ is selected from the group consisting of hydrogen, deuterium, C₁₋₈ alkyl, C₂₋₈ alkenyl, C₃₋₈ cycloalkyl, haloC₁₋₈ alkyl, phenyl, p-methylphenyl, amino, mono C₁₋₈ alkylamino, di C₁₋₈ alkylamino and C₁₋₈ alkanoylamino;
R₅ is selected from the group consisting of hydrogen, deuterium, C₁₋₈ alkyl, C₃₋₈ cycloalkyl, haloC₁₋₈ alkyl and hydroxyC₁₋₈ alkyl;
R₆ is selected from the group consisting of hydrogen, deuterium, C₁₋₈ alkyl, C₁₋₈ alkoxy, C₃₋₈ cycloalkyl, C₃₋₈ cycloalkoxy, haloC₁₋₈ alkyl, haloC₁₋₈ alkoxy, hydroxyC₁₋₈ alkyl and hydroxyC₁₋₈ alkoxy; and
r is 0, 1 or 2.

6. The compound of formula (I), the stereoisomer or the pharmaceutically acceptable salt thereof according to claim 5, which is a compound of the following formula: 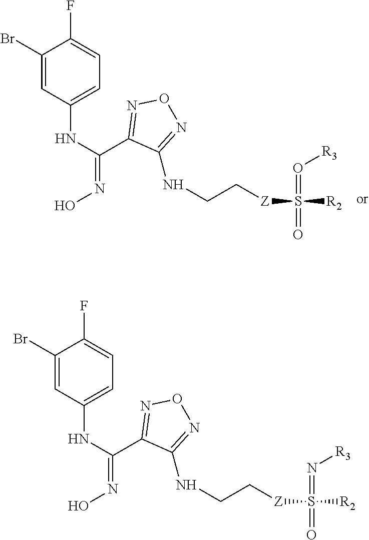
wherein:
Z, R₂, and R₃ are as defined in claim 5.

7. A compound selected from the group consisting of: 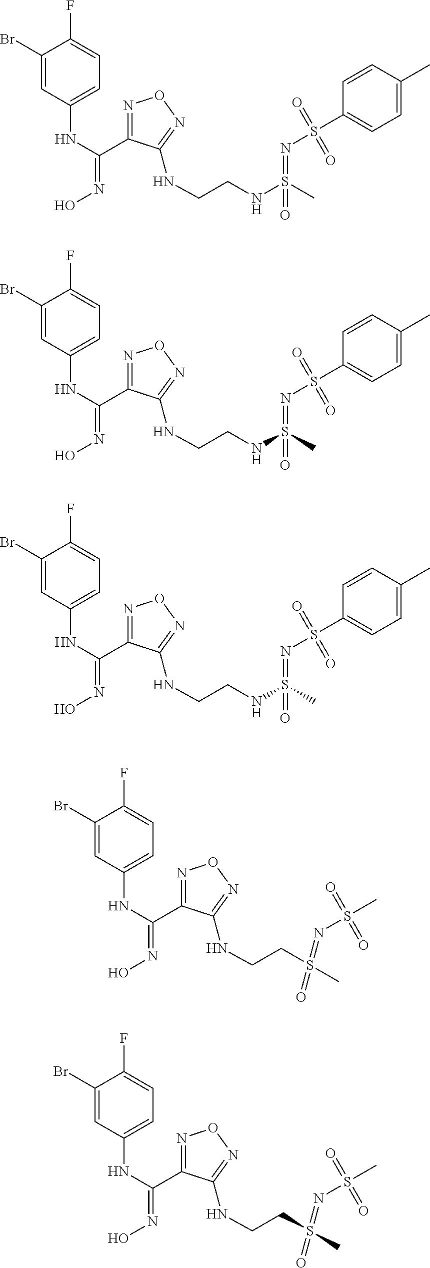 [SMILES: Cc1ccc(S(=O)(=O)N=S(C)(=O)NCCNc2nonc2/C(=N/O)Nc2ccc(F)c(Br)c2)cc1 ; Cc1ccc(S(=O)(=O)N=[S@](C)(=O)NCCNc2nonc2/C(=N/O)Nc2ccc(F)c(Br)c2)cc1 ; Cc1ccc(S(=O)(=O)N=[S@@](C)(=O)NCCNc2nonc2/C(=N/O)Nc2ccc(F)c(Br)c2)cc1 ; CS(=O)(=O)N=S(C)(=O)CCNc1nonc1/C(=N/O)Nc1ccc(F)c(Br)c1 ; CS(=O)(=O)N=[S@](C)(=O)CCNc1nonc1/C(=N/O)Nc1ccc(F)c(Br)c1] 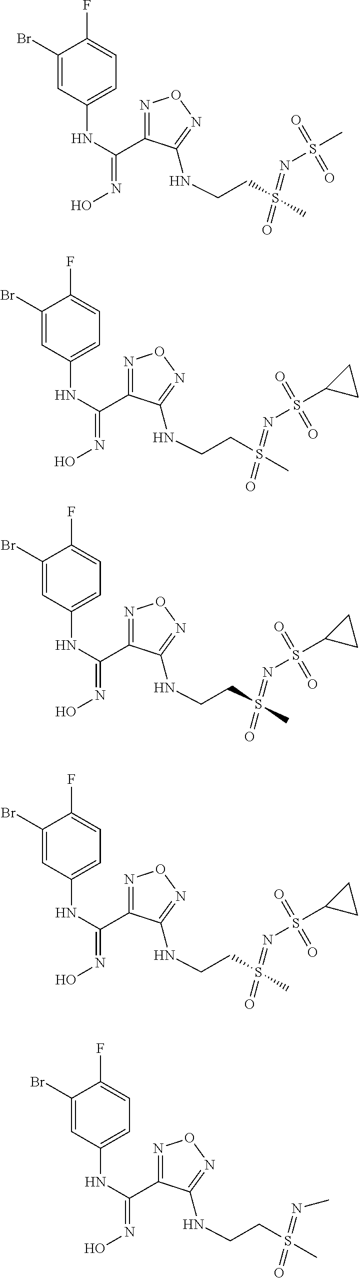 [SMILES: CS(=O)(=O)N=[S@@](C)(=O)CCNc1nonc1/C(=N/O)Nc1ccc(F)c(Br)c1 ; CS(=O)(CCNc1nonc1/C(=N/O)Nc1ccc(F)c(Br)c1)=NS(=O)(=O)C1CC1 ; C[S@@](=O)(CCNc1nonc1/C(=N/O)Nc1ccc(F)c(Br)c1)=NS(=O)(=O)C1CC1 ; C[S@](=O)(CCNc1nonc1/C(=N/O)Nc1ccc(F)c(Br)c1)=NS(=O)(=O)C1CC1 ; CN=S(C)(=O)CCNc1nonc1/C(=N/O)Nc1ccc(F)c(Br)c1] 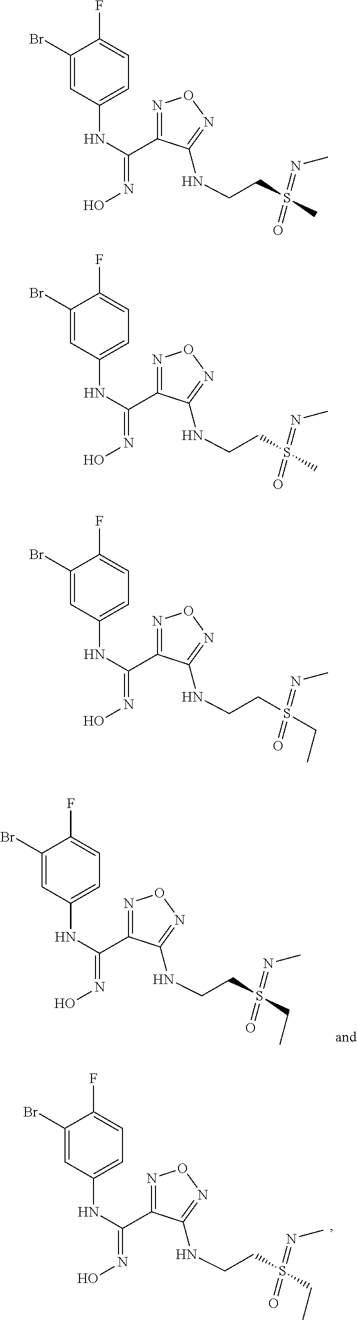 [SMILES: CN=[S@](C)(=O)CCNc1nonc1/C(=N/O)Nc1ccc(F)c(Br)c1 ; CN=[S@@](C)(=O)CCNc1nonc1/C(=N/O)Nc1ccc(F)c(Br)c1 ; CCS(=O)(CCNc1nonc1/C(=N/O)Nc1ccc(F)c(Br)c1)=NC ; CC[S@@](=O)(CCNc1nonc1/C(=N/O)Nc1ccc(F)c(Br)c1)=NC ; CC[S@](=O)(CCNc1nonc1/C(=N/O)Nc1ccc(F)c(Br)c1)=NC]
or a stereoisomer, or a pharmaceutically acceptable salt thereof.

8. An intermediate for preparing the compound of formula (III), the stereoisomer or the pharmaceutically acceptable salt thereof according to claim 5, which is a compound of formula (IV), a stereoisomer or a pharmaceutically acceptable salt thereof: 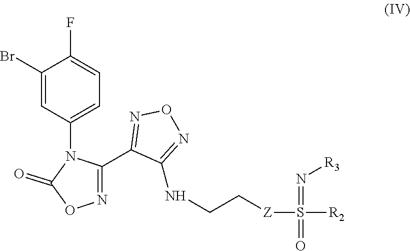
wherein:
Z, R₂, and R₃ are as defined in claim 5.

9. A process for preparing the compound of formula (III), the stereoisomer, or the pharmaceutically acceptable salt thereof according to claim 5, comprising: 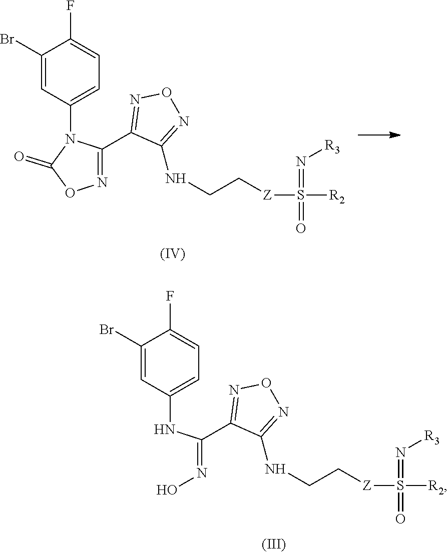
opening a ring of a compound of formula (IV) under an alkaline condition to obtain the compound of formula (III)
wherein:
Z, R₂, and R₃ are as defined in claim 5.

10. A pharmaceutical composition comprising a therapeutically effective amount of the compound of formula (I), the stereoisomer or the pharmaceutically acceptable salt thereof according to claim 1, and a pharmaceutically acceptable carrier.

11.-13. (canceled)

14. A method for modulating the activity of indoleamine 2,3-dioxygenase, comprising contacting the pharmaceutical composition according to claim 10 with indoleamine 2,3-dioxygenase.

15. A method for inhibiting immunosuppression in a subject, comprising administering a therapeutically effective amount of the pharmaceutical composition according to claim 10 to the subject.

16. The method according to claim 14, wherein the modulation is an inhibitory effect.

17. A method for treating or preventing a cancer or tumor, viral infection, depression, neurodegenerative disorder, trauma, age-related cataract, organ transplant rejection or autoimmune disease in a subject, comprising administering to the subject the pharmaceutical composition according to claim 10.

18. The method according to claim 17, wherein the cancer or tumor is selected from the group consisting of lung cancer, bone cancer, gastric cancer, pancreatic cancer, skin cancer, head and neck cancer, uterine cancer, ovarian cancer, testicular cancer, uterine cancer, fallopian tube cancer, endometrial cancer, cervical cancer, vaginal cancer, vulvar cancer, rectal cancer, colon cancer, anal cancer, breast cancer, esophageal cancer, small intestine cancer, endocrine system cancer, thyroid cancer, parathyroid cancer, adrenal cancer, urethral cancer, penile cancer, prostate cancer, pancreatic cancer, brain cancer, testicular cancer, lymph cancer, transitional cell cancer, bladder cancer, kidney or ureter cancer, renal cell carcinoma, renal pelvis cancer, Hodgkin's disease, non-Hodgkin's lymphoma, soft tissue sarcoma, solid tumor in children, lymphocytic lymphoma, central nervous system (CNS) tumor, primary central nervous system lymphoma, tumor angiogenesis, spinal tumor, brainstem glioma, pituitary adenoma, melanoma, Kaposi's sarcoma, epidermoid carcinoma, squamous cell carcinoma, T cell lymphoma, chronic or acute leukemia, and a combination thereof.

19. The method according to claim 14, wherein the pharmaceutical composition is administered in combination with an anti-CTLA-4 antibody, an anti-PD-1 antibody, an anti-PD-L1 antibody, a antiviral agent, a chemotherapeutic agent, an immunosuppressant, radiation, an anti-tumor vaccine, an antiviral vaccine, a cytokine therapy or a tyrosine kinase inhibitor.

20. The method according to claim 15, wherein the pharmaceutical composition is administered in combination with an anti-CTLA-4 antibody, an anti-PD-1 antibody, an anti-PD-L1 antibody, a antiviral agent, a chemotherapeutic agent, an immunosuppressant, radiation, an anti-tumor vaccine, an antiviral vaccine, a cytokine therapy or a tyrosine kinase inhibitor.

21. The method according to claim 17, wherein the pharmaceutical composition is administered in combination with an anti-CTLA-4 antibody, an anti-PD-1 antibody, an anti-PD-L1 antibody, a antiviral agent, a chemotherapeutic agent, an immunosuppressant, radiation, an anti-tumor vaccine, an antiviral vaccine, a cytokine therapy or a tyrosine kinase inhibitor.

22. The method according to claim 19, wherein the cytokine is IL-2, IL-3, IL-4, or IL-5, the chemotherapeutic agent is a cytotoxic agent, and the anti-PD-1 antibody is a pembrolizumab antibody.

23. The compound of formula (I), the stereoisomer or the pharmaceutically acceptable salt thereof according to claim 1, wherein [symbol] is a Z configuration.
